(19) United States
(12) Patent Application Publication
(10) Pub. No.: US 2025/0019760 A1
(43) Pub. Date: Jan. 16, 2025

(54) COMPOSITIONS AND METHODS FOR SEQUENCING MULTIPLE REGIONS OF A TEMPLATE MOLECULE USING ENZYME-BASED REAGENTS

(71) Applicant: Element Biosciences, Inc., San Diego, CA (US)

(72) Inventors: William LIGHT, Poway, CA (US); Hua YU, San Diego, CA (US); Junhua ZHAO, San Diego, CA (US); Su ZHANG, Rosemead, CA (US); Samantha SNOW, San Diego, CA (US); Sinan ARSLAN, San Diego, CA (US); Matthew KELLINGER, San Diego, CA (US); Marco TJIOE, San Diego, CA (US); Scott IM, San Diego, CA (US); James GHADIALI, San Diego, CA (US); Michael KIM, El Cajon, CA (US); Hermes TAYLOR, San Diego, CA (US); Michael PREVITE, San Diego, CA (US); Jake LEVIEUX, San Diego, CA (US); Ramreddy TIPANNA, San Diego, CA (US); Molly HE, San Diego, CA (US)

(21) Appl. No.: 18/654,808

(22) Filed: May 3, 2024

Related U.S. Application Data
(63) Continuation of application No. PCT/US2024/013215, filed on Jan. 26, 2024.
(60) Provisional application No. 63/441,563, filed on Jan. 27, 2023.

(51) Int. Cl. C12Q 1/6874; C12Q 1/44; C12Q 1/6806; C12Q 1/6834; C12Q 1/6876

(52) U.S. Cl. CPC C12Q 1/6874; C12Q 1/44; C12Q 1/6806; C12Q 1/6834; C12Q 1/6876; G01N 2333/922

(57) ABSTRACT

The present disclosure provides compositions comprising enzyme-based reagents, and methods using the enzyme-based reagents, for nucleic acid sequencing. The enzyme-based reagents efficiently remove sequencing read products from a first sequenced region of a template molecule, thereby reducing residual signals in a second sequenced region on the same template molecule.

### CROSS-REFERENCE TO RELATED APPLICATIONS

This application is a continuation application of International Patent Application No. PCT/US2024/013215, filed Jan. 26, 2024 which claims the benefit of and priority to U.S. Provisional Patent Application No. 63/441,563, filed Jan. 27, 2023, the contents of which are incorporated by reference herein in their entirety.

### TECHNICAL FIELD

The present disclosure provides compositions comprising enzyme-based reagents, and methods using the enzyme-based reagents, for nucleic acid sequencing. The enzyme-based reagents efficiently remove sequencing read products from a first sequenced region of a template molecule thereby reducing residual signals in a second sequenced region on the same template molecule.

### BACKGROUND

Polynucleotide sequencing technology has applications in biomedical research and healthcare settings. Improved methods of polynucleotide require enhanced surface chemistry, on-support polynucleotide amplification, and base calling. Currently, these elements produce barriers in existing sequencing technology that result in limits in throughput and poor signal-to-noise ratio, and ultimately to increased costs associated with polynucleotide sequencing.

Thus, there exists a need for new polynucleotide sequencing methods with improved surface chemistry, on-support amplification, and base calling. The present disclosure provides methods and compositions to improve sequencing of polynucleotides by reducing background signal, improving signal-to-noise ratio, and subsequently increasing sequencing quality.

### SUMMARY

In some aspects, provided herein is a method for sequencing a nucleic acid template molecule, comprising: a) providing at least one single-stranded nucleic acid template molecule immobilized to a support wherein the at least one single-stranded nucleic acid template molecule is covalently attached or hybridized to a capture primer which is immobilized to the support; b) hybridizing at least one first sequencing primer to a first region of the nucleic acid template molecule and conducting one or more cycles of sequencing reactions thereby generating at least one first extension duplex each comprising a first sequencing read product hybridized to the template molecule; and c) contacting the first extension duplex with a double-stranded DNA specific enzyme having 5′ to 3′ exonuclease activity under a condition suitable to degrade the first sequencing read product and retain the template molecule thereby removing at least a portion of the first sequencing read product from the first region of the template molecule; and d) washing the retained template molecule at a temperature of 22-27° C. to remove the double-stranded DNA specific enzyme having 5′ to 3′ exonuclease activity.

In some embodiments, the method further comprises: e) hybridizing at least one second sequencing primer to a second region of the same nucleic acid template molecule and conducting one or more sequencing reactions thereby generating a second extension duplex comprising a second sequencing read product hybridized to the template molecule; f) contacting the second extension duplex with a double-stranded DNA specific enzyme having 5′ to 3′ exonuclease activity under a condition suitable to degrade the second sequencing read product and retain the template molecule thereby removing at least a portion of the second sequencing read product from the second region of the template molecule; and g) washing the retained template molecule at a temperature of 22-27° C. to remove the double-stranded DNA specific enzyme having 5′ to 3′ exonuclease activity.

In some embodiments, the method further comprises: h) hybridizing at least one third sequencing primer to a third region of the same nucleic acid template molecule and conducting one or more sequencing reactions thereby generating a third extension duplex comprising a third sequencing read product hybridized to the template molecule; i) contacting the third extension duplex with a double-stranded DNA specific enzyme having 5′ to 3′ exonuclease activity under a condition suitable to degrade the third sequencing read product and retain the template molecule thereby removing at least a portion of the third sequencing read product from the third region of the template molecule; and j) washing the retained template molecule at a temperature of 22-27° C. to remove the double-stranded DNA specific enzyme having 5′ to 3′ exonuclease activity.

In some embodiments, the method further comprises: repeating steps (h), (i) and (j) using a fourth sequencing primer that hybridizes to a fourth region of the same nucleic acid template molecule.

In some embodiments, the method further comprises: repeating steps (h), (i) and (j) using: (i) a fifth sequencing primer that hybridizes to a fifth region of the same nucleic acid template molecule; (ii) a sixth sequencing primer that hybridizes to a sixth region of the same nucleic acid template molecule; (iii) a seventh sequencing primer that hybridizes to a seventh region of the same nucleic acid template molecule; (iv) an eighth sequencing primer that hybridizes to an eighth region of the same nucleic acid template molecule; (v) a ninth sequencing primer that hybridizes to a ninth region of the same nucleic acid template molecule; and/or (vi) a tenth or more sequencing primer that hybridizes to a tenth or more region of the same nucleic acid template molecule.

In some embodiments, conducting one or more sequencing reactions comprises conducting one or more sequencing cycle reactions.

In some embodiments, the double-stranded DNA specific enzyme having 5′ to 3′ exonuclease activity comprises a T7 exonuclease enzyme. In some embodiments, the T7 exonuclease is encoded by T7 phage gene 6.

In some embodiments, the at least one single-stranded nucleic acid template molecule comprises one copy of the sequence-of-interest. In some embodiments, the at least one single-stranded nucleic acid template molecule comprising one copy of the sequence-of-interest is generated via bridge amplification.

In some embodiments, the at least one single-stranded nucleic acid template molecule comprises two or more tandem copies of the sequence-of-interest. In some embodiments, the at least one single-stranded nucleic acid template molecule comprises two or more tandem copies of the sequence-of-interest is generated via rolling circle amplification.

In some embodiments, the at least one single-stranded nucleic acid template molecule comprises at least one uridine nucleotide.

In some embodiments, the at least one single-stranded nucleic acid template molecule lacks a uridine nucleotide.

In some embodiments, the single-stranded nucleic acid template molecule comprises at least one sequence-of-interest and at least one universal adaptor sequence. In some embodiments, the at least one universal adaptor sequence is selected from: a first surface primer binding site (or a complementary sequence thereof) which can hybridize to at least a portion of an immobilized first surface primer; a second surface primer binding site (or a complementary sequence thereof) which can hybridize to at least a portion of an immobilized second surface primer; a first sequencing primer site (or a complementary sequence thereof) which can hybridize to at least a portion of a forward sequencing primer; a second sequencing primer site (or a complementary sequence thereof) which can hybridize to at least a portion of a reverse sequencing primer; a first amplification primer binding site (or a complementary sequence thereof) which can hybridize to at least a portion of a forward amplification primer; a second amplification primer binding site (or a complementary sequence thereof) which can hybridize to at least a portion of a reverse amplification primer; a first sample index sequence; a second sample index sequence; a first compaction oligonucleotide binding site (or a complementary sequence thereof) which can hybridize to at least a portion of a first compaction oligonucleotide; a second compaction oligonucleotide binding site (or a complementary sequence thereof) which can hybridize to at least a portion of a second compaction oligonucleotide; a first unique molecular tag sequence; and/or a second unique molecular tag sequence.

In some embodiments, the single-stranded nucleic acid template molecule comprises any combination of two or more universal adaptor sequences.

In some embodiments, the first sequencing read product of step b) comprises the first sequencing primer joined to a first polynucleotide generated by a polymerase-catalyzed primer extension reaction, wherein the first polynucleotide comprises a sequence complementary to at least a portion of the at least one single-stranded nucleic acid template molecule.

In some embodiments, the second sequencing read product of step d) comprises the second sequencing primer joined to a second polynucleotide generated by a polymerase-catalyzed primer extension reaction, wherein the second polynucleotide comprises a sequence complementary to at least a portion of the at least one single-stranded nucleic acid template molecule.

In some embodiments, the third sequencing read product of step f) comprises the third sequencing primer joined to a third polynucleotide generated by a polymerase-catalyzed primer extension reaction, wherein the third polynucleotide comprises a sequence complementary to at least a portion of the at least one single-stranded nucleic acid template molecule.

In some embodiments, the order of the sequencing the at least one single-stranded nucleic acid template molecule comprises a) sequencing the sequence-of-interest; and b) sequencing the first sample index after sequencing the sequence-of-interest.

In some embodiments, the order of the sequencing the at least one single-stranded nucleic acid template molecule comprises a) sequencing only a portion of the sequence-of-interest; b) sequencing the first sample index after sequencing the sequence-of-interest; and c) sequencing the full length of the sequence-of-interest after sequencing the first sample index.

In some embodiments, the order of the sequencing the at least one single-stranded nucleic acid template molecule comprises a) sequencing the sequence-of-interest; b) sequencing the first sample index after sequencing the sequence-of-interest; and c) sequencing the second sample index after sequencing the first sample index.

In some embodiments, the order of the sequencing the at least one single-stranded nucleic acid template molecule comprises a) sequencing only a portion of the sequence-of-interest; b) sequencing the first sample index after sequencing the sequence-of-interest; c) sequencing the second sample index after sequencing the first sample index; and d) sequencing the full length of the sequence-of-interest after sequencing the second sample index.

In some embodiments, the order of the sequencing the at least one single-stranded nucleic acid template molecule comprises a) sequencing the first sample index; and b) sequencing the sequence-of-interest after sequencing the first sample index.

In some embodiments, the order of the sequencing the at least one single-stranded nucleic acid template molecule comprises a) sequencing the first sample index; and b) sequencing only a portion of the sequence-of-interest after sequencing the first sample index.

In some embodiments, the order of the sequencing the at least one single-stranded nucleic acid template molecule comprises a) sequencing the first sample index; b) sequencing the second sample index after sequencing the first sample index; and c) sequencing the sequence-of-interest after sequencing the second sample index.

In some embodiments, the order of the sequencing the at least one single-stranded nucleic acid template molecule comprises a) sequencing the first sample index; b) sequencing the second sample index after sequencing the first sample index; and c) sequencing only a portion of the sequence-of-interest after sequencing the second sample index.

In some embodiments, the order of the sequencing the at least one single-stranded nucleic acid template molecule comprises a) sequencing the first sample index; b) sequencing the sequence-of-interest after sequencing the first sample index; and c) sequencing the second sample index after sequencing the sequence-of-interest.

In some embodiments, the order of the sequencing the at least one single-stranded nucleic acid template molecule comprises a) sequencing the first sample index; b) sequencing only a portion of the sequence-of-interest after sequencing the first sample index; and c) sequencing the second sample index after sequencing the sequence-of-interest.

In some embodiments, the order of the sequencing the at least one single-stranded nucleic acid template molecule comprises a) sequencing the first sample index; b) sequencing only a portion of the sequence-of-interest after sequencing the first sample index; c) sequencing the second sample index after sequencing the sequence-of-interest; and d) sequencing the full length of the sequence-of-interest after sequencing the second sample index.

In some embodiments, the conducting one or more sequencing reactions comprises conducting one or more cycles of a sequencing reaction with a sequencing polymerase and detectably labeled nucleotide analogs. In some embodiments, the detectably labeled nucleotide analogs comprise nucleotides each comprising an aromatic nucleo-base, a five-carbon sugar moiety, 1-10 phosphate groups, and a fluorophore. In some embodiments, the detectably labeled nucleotide analogs comprise nucleotides each comprise an aromatic nucleo-base, a five-carbon sugar moiety having a chain terminating group at the 3′ carbon sugar position, 1-10 phosphate groups, and a fluorophore.

In some embodiments, the removable chain terminating group of the 3′ carbon sugar position comprises an alkyl group, alkenyl group, alkynyl group, allyl group, aryl group, benzyl group, azide group, azido group, 0-azidomethyl group, amine group, amide group, keto group, isocyanate group, phosphate group, thio group, disulfide group, carbonate group, urea group, or silyl group, and wherein the removable chain terminating moiety is cleavable with a chemical compound to generate an extendible 3′ OH moiety on the sugar group.

In some embodiments, the plurality of detectably labeled nucleotide analogs comprise one type of nucleotide selected from the group consisting of dATP, dGTP, dCTP, dTTP and dUTP. In some embodiments, the plurality of detectably labeled nucleotide analogs comprise a mixture of any combination of two or more types of nucleotides selected from the group consisting of dATP, dGTP, dCTP, dTTP and dUTP.

In some embodiments, the conducting one or more sequencing reactions comprises conducting one or more cycles of a two-stage sequencing reaction, wherein (i) the first stage comprises sequencing with a first plurality of sequencing polymerases and a plurality of detectably labeled multivalent molecules, and (ii) the second stage comprises sequencing with a second plurality of sequencing polymerases and a plurality of unlabeled nucleotide analogs.

In some embodiments, the individual detectably labeled multivalent molecules in the plurality comprise (1) a core, (2) a plurality of nucleotide arms, and (3) at least one fluorophore, wherein individual nucleotide arms comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, and wherein the linker is attached to the nucleotide unit.

In some embodiments, the at least one single-stranded nucleic acid template molecule comprises a concatemer molecule, and wherein the first stage of the two-stage sequencing comprises forming a plurality of binding complexes, which comprises the steps: a) binding a first sequencing primer, a first sequencing polymerase, and a first multivalent molecule to a first portion of the concatemer template molecule thereby forming a first binding complex, wherein a first nucleotide unit of the first multivalent molecule binds to the first sequencing polymerase; and b) binding a second sequencing primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the binding of steps (a) and (b) are conducted under a condition suitable to inhibit polymerase-catalyzed incorporation of the bound first and second nucleotide units in the first and second binding complexes respectively, wherein the first and second binding complexes which includes the same multivalent molecule forms an avidity complex; c) detecting the first and second binding complexes on the same concatemer template molecule; and d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule.

### DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic showing an exemplary single stranded template molecule comprising: a second surface primer binding site (e.g., SP2; surface pinning primer binding site); a second index sequence; a first sequencing primer binding site (e.g., a forward sequencing primer binding site); a sequence-of-interest (e.g., an insert); a second sequencing primer binding site (e.g., a reverse sequencing primer binding site); a first index sequence; and a first surface primer binding site (e.g., SP1; a capture primer binding site). In some embodiments, the template molecule shown in FIG. 1 comprises a one copy template molecule having one copy of the sequence-of-interest and one copy of various universal adaptor sequences. In some embodiments, the template molecule shown in FIG. 1 is one unit of a concatemer having two or more tandem copies of the unit, wherein each unit comprises a sequence-of-interest and one copy of various universal adaptor sequences.

FIG. 2 is a schematic showing an exemplary single stranded template molecule comprising: a second surface primer binding site (e.g., SP2; surface pinning primer binding site); a first sequencing primer binding site (e.g., a forward sequencing primer binding site); a second index sequence; a sequence-of-interest (e.g., an insert); a first index sequence; a second sequencing primer binding site (e.g., a reverse sequencing primer binding site); and a first surface primer binding site (e.g., SP1; a capture primer binding site). In some embodiments, the template molecule shown in FIG. 1 comprises a one copy template molecule having one copy of the sequence-of-interest and one copy of various universal adaptor sequences. In some embodiments, the template molecule shown in FIG. 1 is one unit of a concatemer having two or more tandem copies of the unit, wherein each unit comprises a sequence-of-interest and one copy of various universal adaptor sequences.

FIG. 3 is a schematic showing an exemplary order of sequencing: Read 1 (e.g., full length sequencing the sequence-of-interest); first sample index; and second sample index.

FIG. 4 is a schematic showing an exemplary order of sequencing: Read 1 (e.g., sequencing only a portion of the sequence-of-interest; first sample index; second sample index; and Read 1 (e.g., sequencing full length the sequence-of-interest).

FIG. 5 is a schematic showing an exemplary order of sequencing: First sample index; second sample index; Read 1 (e.g., full length sequencing the sequence-of-interest).

FIG. 6 is a schematic showing an exemplary order of sequencing: First sample index; Read 1 (e.g., sequencing only a portion of the sequence-of-interest; second sample index; and Read 1 (e.g., full length sequencing the sequence-of-interest).

FIG. 7 is a schematic showing an exemplary order of sequencing for a pairwise workflow: Forward first sample index; forward second sample index; forward Read 1 (e.g., full length sequencing the sequence-of-interest); pairwise turn; reverse Read 2 (e.g., full length sequencing the sequence-of-interest).

FIG. 8 is a schematic showing an exemplary order of sequencing for a pairwise workflow: Forward Read 1 (e.g., full length sequencing the sequence-of-interest); pairwise turn; reverse first sample index; reverse second sample index; and reverse Read 2 (e.g., full length sequencing the sequence-of-interest).

FIG. 9A is a schematic showing an exemplary single stranded nucleic acid concatemer template molecule immobilized to an immobilized first surface primer. The concatemer template molecule is covalently attached to the immobilized first surface primer. The immobilized concatemer template molecule comprises at least one nucleotide having a scissile moiety that can be cleaved to generate an abasic site in the immobilized concatemer template molecule. In some embodiments, the immobilized concatemer template molecule can be generated by conducting an on-support rolling circle amplification reaction. The arrangement of the sequence-of-interest and various universal adaptor sequences is for illustration purposes. The skilled artisan will appreciate that many other arrangements are possible. FIGS. 9A-G show the workflow of pairwise sequencing the immobilized concatemer template molecule depicted in FIG. 9A.

FIG. 9B is a schematic showing an exemplary forward sequencing reaction conducted on the immobilized concatemer template molecule shown in FIG. 9A. The forward sequencing reaction can be conducted with a plurality of soluble forward sequencing primers and generates a plurality of first forward sequencing read products. The immobilized concatemer template molecule can have two or more first forward sequencing read products hybridized thereon.

FIG. 9C is a schematic showing an exemplary method for replacing the first forward sequencing read products by conducting a primer extension reaction with a strand displacing polymerase in the absence of an added soluble primer thereby generating a forward extension strand. The strand displacing polymerase can use an upstream first forward sequencing read product to initiate a primer extension reaction.

FIG. 9D is a schematic showing an exemplary method for replacing the first forward sequencing read products by removing the first forward sequencing read products, and conducting a primer extension reaction with a new soluble forward sequencing primer thereby generating a forward extension strand.

FIG. 9E is a schematic showing an exemplary method for generating abasic sites in the immobilized single stranded concatemer template molecule at the nucleotides having the scissile moiety and generating gaps at the abasic sites to generate a plurality of gap-containing concatemer template molecules while retaining the forward extension strand and retaining the immobilized first surface primer. The forward extension strand can be generated for example by the methods depicted in FIG. 9C or 9D.

FIG. 9F is a schematic showing an exemplary retained forward extension strand after removal of the gap-containing concatemer template molecule as shown in FIG. 9E.

FIG. 9G is a schematic showing an exemplary reverse sequencing reaction conducted on the retained forward extension strand shown in FIG. 9F. The reverse sequencing reaction can be conducted with a plurality of soluble reverse sequencing primers. The retained forward extension strand can have two or more first reverse sequencing read products hybridized thereon. The first reverse sequencing read products are not hybridized to the first surface primer, or covalently joined to the first surface primer. Therefore, the first reverse sequencing read products are not immobilized to the support. For the sake of simplicity, FIGS. 9A-E show an exemplary immobilized concatemer molecule with four copies of the sequence-of-interest and various universal primer binding sites. The skilled artisan will appreciate that the immobilized concatemer molecule can include more than four tandem copies of a unit where each unit includes the sequence-of-interest and various universal primer binding sites.

FIG. 9H is a schematic showing an exemplary support having a first and second surface primers immobilized thereon. A portion of the immobilized concatemer template molecule shown in FIG. 9A is hybridized to the immobilized second surface primer. The immobilized second surface primers serve to pin down a portion of the immobilized concatemer template molecules to the support. The immobilized concatemer template molecule has two or more copies of a universal binding sequence for an immobilized second surface primer. The portion of the immobilized concatemer template molecule that includes the universal binding sequence for an immobilized second surface primer can hybridize to the immobilized second surface primer.

FIG. 10 is a schematic showing an exemplary single stranded nucleic acid concatemer template molecule immobilized to an immobilized first surface primer. The concatemer template molecule is hybridized to the immobilized first surface primer. The immobilized concatemer template molecule comprises at least one nucleotide having a scissile moiety that can be cleaved to generate an abasic site in the immobilized concatemer template molecule. In some embodiments, the immobilized concatemer template molecule can be generated by conducting an in-solution rolling circle amplification reaction and distributing the rolling circle amplification reaction onto the support. The arrangement of the sequence-of-interest and various universal adaptor sequences is for illustration purposes. The skilled artisan will appreciate that many other arrangements are possible. The immobilized concatemer template molecule can be subjected to the pairwise sequencing workflow that is depicted in FIGS. 9A-G.

FIG. 11 shows two sets of bar graphs which compare full width half maximum (FWHM) measurements from polonies (e.g., concatemers) immobilized to a support and subjected to repeat cycles of: hybridizing a sequencing primer to form template/primer duplexes, binding the template/primer duplexes to a sequencing polymerase and detectably labeled multivalent molecules to form detectable complex, four channel imaging the fluorescent signals from the detectable complex, and dehybridizing the detectable complex using either a conventional dehybridization reagent or an enzyme-based dehybridization reagent. The polonies that were subjected to repeat cycles of hybridization and dehybridization using a conventional dehybridization reagent are shown on the Left. The polonies that were subjected to repeat cycles of hybridization and dehybridization using an enzyme-based dehybridization reagent are shown on the Right.

FIG. 12 shows two sets of bar graphs which compare P90 measurements (e.g., intensity) from polonies (e.g., concatemers) immobilized to a support and subjected to repeat cycles of: hybridizing a sequencing primer to form template/primer duplexes, binding the template/primer duplexes to a sequencing polymerase and detectably labeled multivalent molecules to form detectable complex, four channel imaging the fluorescent signals from the detectable complex, and dehybridizing the detectable complex using either a conventional dehybridization reagent or an enzyme-based dehybridization reagent. The polonies that were subjected to repeat cycles of hybridization and dehybridization using a conventional dehybridization reagent is shown on the Left. The polonies that were subjected to repeat cycles of hybridization and dehybridization using an enzyme-based dehybridization reagent is shown on the Right.

FIG. 13 shows two sets of bar graphs which compare percent signal remaining after dehybridization from polonies (e.g., concatemers) immobilized to a support and subjected to repeat cycles of: hybridizing a sequencing primer to form template/primer duplexes, binding the template/primer duplexes to a sequencing polymerase and detectably labeled multivalent molecules to form detectable complex, four channel imaging the fluorescent signals from the detectable complex, and dehybridizing the detectable complex using either a conventional dehybridization reagent or an enzyme-based dehybridization reagent. The polonies that were subjected to repeat cycles of hybridization and dehybridization using a conventional dehybridization reagent is shown on the Left. The polonies that were subjected to repeat cycles of hybridization and dehybridization using an enzyme-based dehybridization reagent is shown on the Right.

FIG. 14 shows a series of fluorescent images of polonies (e.g., concatemers) immobilized to a support and subjected to repeat cycles of: hybridizing a sequencing primer to form template/primer duplexes, binding the template/primer duplexes to a sequencing polymerase and detectably labeled multivalent molecules to form detectable complex, imaging the fluorescent signals from the detectable complexes, and dehybridizing the detectable complex using a conventional dehybridization reagent. A significant level of signal was detected in the first, second and third dehybridization images. The level of signal diminishes in the second, third and fourth hybridization images.

FIG. 15 shows a series of fluorescent images of polonies (e.g., concatemers) immobilized to a support and subjected to repeat cycles of hybridizing a sequencing primer to form template/primer duplexes, binding the template/primer duplexes to a sequencing polymerase and detectably labeled multivalent molecules to form detectable complex, imaging the fluorescent signals from the detectable complexes, and dehybridizing the detectable complex using an enzyme-based dehybridization reagent. A significantly reduced level of signal was detected in the first, second and third dehybridization images compared to the dehybridization signals detected in FIG. 14. For the enzyme-based cycles, the level of signal in the second, third and fourth hybridization images does not significantly diminish compared to the hybridization signals detected in FIG. 14.

FIG. 16 is a schematic of an exemplary low binding support comprising a glass substrate and alternating layers of hydrophilic coatings which are covalently or non-covalently adhered to the glass, and which further comprises chemically-reactive functional groups that serve as attachment sites for oligonucleotide primers (e.g., capture oligonucleotides). In an alternative embodiment, the support can be made of any material, such as glass, plastic or a polymer material.

FIG. 17 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 18 is a schematic of an exemplary multivalent molecule comprising a generic core attached to a plurality of nucleotide-arms.

FIG. 19 is a schematic of an exemplary multivalent molecule comprising a dendrimer core attached to a plurality of nucleotide-arms.

FIG. 20 shows a schematic of an exemplary multivalent molecule comprising a core attached to a plurality of nucleotide-arms, where the nucleotide arms comprise biotin, spacer, linker and a nucleotide unit.

FIG. 21 is a schematic of an exemplary nucleotide-arm comprising a core attachment moiety, spacer, linker and nucleotide unit.

FIG. 22 shows the chemical structure of an exemplary spacer (top), and the chemical structures of various exemplary linkers, including an 11-atom Linker, 16-atom Linker, 23-atom Linker and an N3 Linker (bottom).

FIG. 23 shows the chemical structures of various exemplary linkers, including Linkers 1-9.

FIG. 24A shows the chemical structures of various exemplary linkers joined/attached to nucleotide units.

FIG. 24B shows the chemical structures of various exemplary linkers joined/attached to nucleotide units.

FIG. 24C shows the chemical structures of various exemplary linkers joined/attached to nucleotide units.

FIG. 25 shows the chemical structure of an exemplary biotinylated nucleotide-arm. In this example, the nucleotide unit is connected to the linker via a propargyl amine attachment at the 5 position of a pyrimidine base or the 7 position of a purine base.

### DETAILED DESCRIPTION

### Definitions

The headings provided herein are not limitations of the various aspects of the disclosure, which aspects can be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about” and “approximately” refer to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Also, where ranges and/or subranges of values are provided, the ranges and/or subranges can include the endpoints of the ranges and/or subranges.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

As used herein, the “nucleic acid(s) of interest” can be extracted from cells or cellular biological samples using any of a number of techniques known to those of skill in the art. For example, a typical DNA extraction procedure comprises (i) collection of the cell sample or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis. A variety of suitable commercial nucleic acid extraction and purification kits are consistent with the disclosure herein. Examples include, but are not limited to, the QIAamp® kits (for isolation of genomic DNA from human samples) and DNAeasy® kits (for isolation of genomic DNA from animal or plant samples) from Qiagen® (Germantown, MD), or the Maxwell® and ReliaPrep™ series of kits from Promega® (Madison, WI).

As used herein, the term “cellular biological sample” refers to a single cell, a plurality of cells, a tissue, an organ, an organism, or section of any of these cellular biological samples. The cellular biological sample can be extracted (e.g., biopsied) from an organism, or obtained from a cell culture grown in liquid or in a culture dish. The cellular biological sample can comprise a sample that is fresh, frozen, fresh frozen, or archived (e.g., formalin-fixed paraffin-embedded; FFPE). The cellular biological sample can be embedded in a wax, resin, epoxy or agar. The cellular biological sample can be fixed, for example and without limitation, in any one or any combination of two or more of acetone, ethanol, methanol, formaldehyde, paraformaldehyde-Triton or glutaraldehyde. Other methods of fixation are known in the art. The cellular biological sample can be sectioned or non-sectioned. The cellular biological sample can be stained, de-stained, or non-stained.

The term “polymerase” and its variants, as used herein, comprises an enzyme comprising a domain that binds a nucleotide (or nucleoside) where the polymerase can form a complex having a template nucleic acid and a complementary nucleotide. The polymerase can have one or more activities including, but not limited to, base analog detection activities, DNA polymerization activity, reverse transcription activity, DNA binding, strand displacement activity, and nucleotide binding and recognition. A polymerase can be any enzyme that can catalyze polymerization of nucleotides (including analogs thereof) into a nucleic acid strand. Typically, but not necessarily, such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include, for example and without limitation, naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). The polymerase includes catalytically inactive polymerases, catalytically active polymerases, reverse transcriptases, and other enzymes comprising a nucleotide binding domain. In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase.

As used herein, the term “strand displacing” refers to the ability of a polymerase to locally separate strands of double-stranded nucleic acids and synthesize a new strand in a template-based manner. Strand displacing polymerases can displace a complementary strand from a template strand and catalyze new strand synthesis. Strand displacing polymerases can include mesophilic and thermophilic polymerases. Strand displacing polymerases include wild type enzymes and variants thereof, including exonuclease minus mutants, mutant versions, chimeric enzymes and truncated enzymes. Examples of strand displacing polymerases include, for example and without limitation, phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent DNA polymerase and KOD DNA polymerase. The phi29 DNA polymerase can be wild type phi29 DNA polymerase (e.g., MagniPhi™ from Expedeon®), or variant EquiPhi29 DNA polymerase (e.g., from Thermo Fisher Scientific®), or chimeric QualiPhi® DNA polymerase (e.g., from 4basebio®).

The terms “nucleic acid”, “polynucleotide” and “oligonucleotide” and other related terms as used herein are used interchangeably and refer to polymers of nucleotides and are not limited to any particular length. Nucleic acids include recombinant and chemically-synthesized forms. Nucleic acids can be isolated. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids can comprise polymers of nucleotides, where the nucleotides include natural or non-natural bases and/or sugars. Nucleic acids can comprise naturally-occurring internucleosidic linkages, for example, phosphdiester linkages. Nucleic acids comprise non-natural internucleoside linkages, including phosphorothioate, phosphorothiolate, or peptide nucleic acid (PNA) linkages. In some embodiments, nucleic acids comprise one type of polynucleotides, or a mixture of two or more different types of polynucleotides.

The term “operably linked” and “operably joined” or related terms as used herein refers to juxtaposition of components. The juxtapositioned components can be linked together covalently. For example, and without limitation, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, and without limitation, linkage between a primer binding sequence and a sequence of interest forms a nucleic acid library molecule having a portion that can bind to a primer. In another non-limiting example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

As used herein, the terms “linked”, “joined”, “attached”, “appended” and variants thereof comprise any type of fusion, bond, adherence or association between any combination of compounds or molecules that is of sufficient stability to withstand use in a particular procedure. The procedure can include, but is not limited to: nucleotide binding; nucleotide incorporation; de-blocking (e.g., removal of chain-terminating moiety); washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “primer” and related terms as used herein refers to an oligonucleotide that is capable of hybridizing with a DNA and/or RNA polynucleotide template to form a duplex molecule. Primers can be single-stranded along their entire length or have single-stranded and double-stranded portions. Primers can comprise natural nucleotides and/or nucleotide analogs. Primers can be recombinant nucleic acid molecules. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-catalyzed primer extension reaction. Alternatively, the 3′ end of the primer can lack a 3′ OH moiety, or can include a terminal 3′ blocking group that inhibits nucleotide polymerization in a polymerase-catalyzed reaction. Any one nucleotide, or more than one nucleotide, along the length of the primer can be labeled with a detectable reporter moiety. A primer can be in solution (e.g., a soluble primer) or can be immobilized to a support (e.g., a capture primer).

As used herein, the term “template nucleic acid”, “template polynucleotide”, “target nucleic acid” “target polynucleotide”, “template strand” and other variations refer to a nucleic acid strand that serves as the basis nucleic acid molecule for any of the amplification and/or sequencing methods described herein. The template nucleic acid can be single-stranded or double-stranded, or the template nucleic acid can have single-stranded or double-stranded portions. The template nucleic acid can be obtained from a naturally-occurring source, recombinant form, or chemically synthesized to include any type of nucleic acid analog. The template nucleic acid can be linear, concatemeric, circular, or other forms.

As used herein, the term “adaptor” and related terms refers to oligonucleotides that can be operably linked (appended) to a target polynucleotide, where the adaptor confers a function to the co-joined adaptor-target molecule. Adaptors comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. At least a portion of the adaptors comprise a known and pre-determined sequence. An adaptor can include a sequence that is complementary to at least a portion of a primer, such as an amplification primer, a sequencing primer, or a capture primer (e.g., soluble or immobilized capture primers). Adaptors can include a random sequence or a degenerate sequence. Adaptors can include at least one inosine residue. Adaptors can include at least one phosphorothioate, phosphorothiolate and/or phosphoramidate linkage. Adaptors can include at least one barcode sequence which can be used to distinguish polynucleotides (e.g., insert sequences) from different sample sources in a multiplex assay. Adaptors can include at least one unique identification sequence (e.g., a molecular tag) that can be used to uniquely identify a nucleic acid molecule to which the adaptor is appended. In some embodiments, the unique identification sequence comprises 2-12 or more nucleotides having a known sequence. For example, the unique identification sequence comprises a known random sequence where a nucleotide at each position is randomly selected from nucleotides having a base A, G, C, T or U. Adaptors can include at least one restriction enzyme recognition sequence, including any one or any combination of two or more selected from a group consisting of type I, type II, type III, type IV, type Hs or type IIB.

As used herein, the term “universal sequence” and related terms refers to a sequence in a nucleic acid molecule that is common among two or more polynucleotide molecules. For example, an adaptor having a universal sequence can be operably joined to a plurality of polynucleotides so that the population of co-joined molecules carry the same universal adaptor sequence. Examples of universal adaptor sequences include, without limitation, an amplification primer sequence, a sequencing primer sequence or a capture primer sequence (e.g., soluble or immobilized capture primers).

When used herein in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing can be the standard A-T or C-G base pairing, or can be other forms of base-pairing interactions. Duplex nucleic acids can include mismatched base-paired nucleotides.

When used herein in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “nucleotides” and related terms, as used herein, refers to a molecule comprising an aromatic base, a five-carbon sugar (e.g., ribose or deoxyribose), and at least one phosphate group. Canonical or non-canonical nucleotides are consistent with use of the term. In some embodiments, the nucleotide comprises a monophosphate, diphosphate, or triphosphate, or corresponding phosphate analog. The term “nucleoside” refers to a molecule comprising an aromatic base and a sugar. Nucleotides and nucleosides can be non-labeled or labeled with a detectable reporter moiety.

Nucleotides (and nucleosides) typically comprise a heterocyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening 0, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

As used herein, a “nucleotide unit” or ‘nucleotide moiety” refers to nucleotides (e.g., dATP, dTTP, dGTP, dCTP, or dUTP), or analogs thereof, comprising comprises a base, sugar and at least one phosphate group. Nucleotide units can be attached to the multivalent molecules used in the sequencing reactions described herein. In general, all nucleotide units attached to the same multivalent molecule will have the same identity (e.g., all A, all T, all C, or all G), although the skilled artisan will appreciate that there may be situations in which a multivalent molecule comprising nucleotide units of differing identity will be advantageous.

As used herein, the term “rolling circle amplification” generally refers to an amplification method that employs a circularized nucleic acid template molecule containing a target sequence of interest, an amplification primer binding sequence, and optionally one or more adaptor sequences such as a sequencing primer binding sequence and/or a sample index sequence. The rolling circle amplification reaction can be conducted under isothermal amplification conditions, and includes the circularized nucleic acid template molecule, an amplification primer, a strand-displacing polymerase and a plurality of nucleotides, to generate a concatemer containing tandem repeat sequences of the circular template molecule and any adaptor sequences present in the original circularized nucleic acid template molecule. The concatemer can self-collapse to form a nucleic acid nanoball. The shape and size of the nanoball can be further compacted by including a pair of inverted repeat sequences in the circular template molecule, or by conducting the rolling circle amplification reaction with one or more compaction oligonucleotides. One of the advantages of using rolling circle amplification to generate clonal amplicons for a sequencing workflow, is that the repeat copies of the target sequence in the nanoball can be simultaneously sequenced to increase signal intensity. In some embodiments, the rolling circle amplification reaction can be conducted in the presence of a plurality of compaction oligonucleotides having at least four consecutive guanines. The rolling circle amplification reaction generates concatemers comprising repeat copies of the universal binding sequence for the compaction oligonucleotide. At least one compaction oligonucleotide can form a guanine tetrad and hybridize to the universal binding sequences for the compaction oligonucleotide, and the resulting concatemer can fold to form an intramolecular G-quadruplex structure. The concatemers can self-collapse to form compact nanoballs. Formation of the guanine tetrads and G-quadruplexes in the nanoballs may increase the stability of the nanoballs to retain their compact size and shape which can withstand repeated flows of reagents for conducting any of the sequencing workflows described herein.

When used herein in reference to nucleic acids, the terms “amplify”, “amplifying”, “amplification”, and other related terms include producing multiple copies of an original polynucleotide template molecule, where the copies comprise a sequence that is complementary to the template sequence, and/or the copies comprise a sequence that is the same as the template sequence. In some embodiments, the copies comprise a sequence that is substantially identical to a template sequence, and/or is substantially identical to a sequence that is complementary to the template sequence.

As used herein, the term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) can comprise a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene)hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene)hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a FRET pair, such that multiple classifications can be performed under a single excitation and imaging step. As used herein, FRET may comprise excitation exchange (Forster) transfers, or electron-exchange (Dexter) transfers.

The term “persistence time” and related terms as used herein refers to the length of time that a binding complex remains stable without dissociation of any of the components, where the components of the binding complex include a nucleic acid template and nucleic acid primer, a polymerase, a nucleotide unit of a multivalent molecule or a free (e.g., unconjugated) nucleotide. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset and/or duration of a binding complex, such as by observing a signal from a labeled component of the binding complex. For example, and without limitation, a labeled nucleotide or a labeled reagent comprising one or more nucleotides may be present in a binding complex, thus allowing the signal from the label to be detected during the persistence time of the binding complex. One exemplary label is a fluorescent label. The binding complex (e.g., ternary complex) remains stable until subjected to a condition that causes dissociation of interactions between any of the polymerase, template molecule, primer and/or the nucleotide unit or the nucleotide. For example, and without limitation, a dissociating condition comprises contacting the binding complex with any one or any combination of a detergent, EDTA and/or water.

The term “support” as used herein refers to a substrate that is designed for deposition of biological molecules or biological samples for assays and/or analyses. Examples of biological molecules to be deposited onto a support include nucleic acids (e.g., DNA, RNA, or a combination thereof), polypeptides, saccharides, lipids, a single cell or multiple cells. Examples of biological samples include but are not limited to saliva, phlegm, mucus, blood, plasma, serum, urine, stool, sweat, tears and fluids from tissues or organs.

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example, comprising a capillary or an interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. In some embodiments, the support comprises a polymer, e.g., a synthetic polymer. In some embodiments, the support comprises glass. In some embodiments, the support comprises plastic. Various compositions of both glass and plastic substrates are contemplated.

In some aspects, the present disclosure provides a plurality (e.g., two or more) of nucleic acid template molecules immobilized to a support. In some embodiments, the immobilized plurality of nucleic acid template molecules has the same sequence or each has a different sequence. In some embodiments, individual nucleic acid template molecules in the plurality of nucleic acid template molecules are immobilized to a different site on the support. In some embodiments, two or more individual nucleic acid template molecules in the plurality of nucleic acid templates are immobilized to a site on the support.

As used herein, the term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) are immobilized with nucleic acid template molecules to form a nucleic acid template array. In some embodiments, the nucleic acid template molecules that are immobilized at a plurality of pre-determined sites by hybridization to immobilized surface capture primers, or the nucleic acid template molecules are covalently attached to the surface capture primers. In some embodiments, the nucleic acid template molecules that are immobilized at a plurality of pre-determined sites, for example immobilized at 10²-10¹⁵ sites (e.g., 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, 10¹³ sites, 10¹⁴ sites, or 10¹⁵ sites) or more. In some embodiments, the immobilized nucleic acid template molecules are clonally-amplified to generate immobilized nucleic acid polonies at the plurality of pre-determined sites. In some embodiments, individual immobilized nucleic acid polonies comprise linear one copy molecules, or comprise single-stranded or double-stranded concatemers.

In some embodiments, a support comprising a plurality of sites located at random locations on the support is referred to herein as a support having randomly located sites thereon. The location of the randomly located sites on the support may not be pre-determined. The plurality of randomly-located sites can be arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more (e.g. 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, 10¹³ sites, 10¹⁴ sites, 10¹⁵ sites, or more)) are immobilized with nucleic acid template molecules. In some embodiments, the nucleic acid template molecules are immobilized at a plurality of randomly located sites by hybridization to immobilized surface capture primers, or the nucleic acid template molecules are covalently attached to the surface capture primers. In some embodiments, the nucleic acid templates that are immobilized at a plurality of randomly located sites, for example, immobilized at 10²-10¹⁵ sites (e.g., 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, 10¹³ sites, 10¹⁴ sites, 10¹⁵ sites) or more. In some embodiments, the immobilized nucleic acid templates are clonally-amplified to generate immobilized nucleic acid polonies at the plurality of randomly located sites. In some embodiments, individual immobilized nucleic acid polonies comprise linear one copy molecules, or comprise single-stranded or double-stranded concatemers.

In some embodiments, the plurality of immobilized surface capture primers on the support (e.g., located at pre-determined or random locations on the support) are in fluid communication with each other to permit flowing a solution of reagents (e.g., nucleic acid template molecules, soluble primers, enzymes, nucleotides, divalent cations, buffers, and the like) onto the support so that the plurality of immobilized surface capture primers on the support can be essentially simultaneously reacted with the reagents in a massively parallel manner. In some embodiments, the fluid communication of the plurality of immobilized surface capture primers can be used to conduct nucleic acid amplification reactions (e.g., RCA, MDA, PCR and bridge amplification) essentially simultaneously on the plurality of immobilized surface capture primers.

In some embodiments, the plurality of immobilized nucleic acid polonies on the support are in fluid communication with each other to permit flowing a solution of reagents (e.g., enzymes, nucleotides, divalent cations, and the like) onto the support so that the plurality of immobilized nucleic acid polonies on the support can be essentially simultaneously reacted with the reagents in a massively parallel manner. In some embodiments, the fluid communication of the plurality of immobilized nucleic acid polonies can be used to conduct nucleotide binding assays and/or conduct nucleotide polymerization reactions (e.g., primer extension or sequencing) essentially simultaneously on the plurality of immobilized nucleic acid polonies, and optionally to conduct detection and imaging for massively parallel sequencing.

In some embodiments, the term “immobilized” and related terms refer to nucleic acid molecules that are attached to a support through covalent bond or non-covalent interaction, or attached to a coating on the support, or buried within a matrix formed by a coating on the support, where the nucleic acid molecules include surface capture primers, nucleic acid template molecules and extension products of capture primers. Extension products of capture primers can include nucleic acid concatemers (e.g., nucleic acid polonies). The nucleic acid molecules can be immobilized at pre-determined or random locations on the support. The nucleic acid molecules can be immobilized at pre-determined or random locations on or within a coating passivated on the support.

In some embodiments, the term “immobilized” and related terms refer to enzymes (e.g., polymerases) that are attached to a support through covalent bond or non-covalent interaction, or attached to a coating on the support, or buried within a matrix formed by a coating on the support. The enzymes can be immobilized at pre-determined or random locations on the support. The enzymes can be immobilized at pre-determined or random locations on or within a coating passivated on the support.

In some embodiments, one or more nucleic acid template molecules are immobilized on the support, for example immobilized at the sites on the support. In some embodiments, the one or more nucleic acid template molecules are clonally-amplified. In some embodiments, the one or more nucleic acid template molecules are clonally-amplified off the support (e.g., in-solution). In some embodiments, following clonal amplification, the one or more nucleic acid template molecules are deposited onto the support and immobilized on the support. In some embodiments, the clonal amplification reaction of the one or more nucleic acid template molecules is conducted on the support resulting in immobilization on the support. In some embodiments, the one or more nucleic acid template molecules are clonally-amplified (e.g., in solution or on the support) using a nucleic acid amplification reaction. In some embodiments, the nucleic acid amplification reaction includes any one or any combination of: polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, and/or single-stranded binding (SSB) protein-dependent amplification. Other suitable methods of nucleic acid amplification are known in the art.

The term “surface primer” and related terms refers to single-stranded oligonucleotides that are immobilized to a support and comprise a sequence that can hybridize to at least a portion of a nucleic acid template molecule. Surface capture primers can be used to immobilize template molecules to a support via hybridization. Surface capture primers can be immobilized to a support in a manner that resists primer removal during flowing, washing, aspirating, and changes in temperature, pH, salts, chemical and/or enzymatic conditions. Typically, but not necessarily, the 5′ end of a surface capture primer can be immobilized to a support or to a coating on the support (or embedded in a coating on the support). Alternatively, an interior portion or the 3′ end of a surface capture primer can be immobilized to a support.

The sequence of surface capture primers can be wholly or partially complementary along their length to at least a portion of the nucleic acid template molecule. A support can include a plurality of immobilized surface capture primers having the same sequence, or having two or more different sequences. Surface capture primers can be any length, for example 4-50 nucleotides, or 50-100 nucleotides, or 100-150 nucleotides, or longer lengths. The skilled artisan will appreciate suitable surface capture primer lengths dependent upon, e.g., the template molecule, properties of the surface capture primer, etc.

A surface capture primer can have a terminal 3′ nucleotide having a sugar 3′ OH moiety which is extendible for nucleotide polymerization (e.g., polymerase catalyzed polymerization). A surface capture primer can have a terminal 3′ nucleotide having the 3′ sugar position linked to a chain-terminating moiety that inhibits nucleotide polymerization. The 3′ chain-terminating moiety can be removed (e.g., de-blocked) to convert the 3′ end to an extendible 3′ OH end using a de-blocking agent. Examples of chain terminating moieties include, without limitation, alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. Azide type chain terminating moieties include, without limitation, azide, azido and azidomethyl groups. Examples of de-blocking agents can include a phosphine compound, such as Tris(2-carboxyethyl)phosphine (TCEP) and bis-sulfo triphenyl phosphine (BS-TPP), for chain-terminating groups azide, azido and azidomethyl groups. Examples of de-blocking agents can include tetrakis(triphenylphosphine)palladium(0) (Pd(PPh₃)₄) with piperidine, or with 2,3-Dichloro-5,6-dicyano-1,4-benzo-quinone (DDQ), for chain-terminating groups alkyl, alkenyl, alkynyl and allyl. Examples of a de-blocking agent can include Pd/C for chain-terminating groups aryl and benzyl. Examples of de-blocking agents can include phosphine, beta-mercaptoethanol or dithiothritol (DTT), for chain-terminating groups amine, amide, keto, isocyanate, phosphate, thio and disulfide. Examples of de-blocking agents can include potassium carbonate (K₂CO₃) in MeOH, triethylamine in pyridine, and Zn in acetic acid (AcOH), for carbonate chain-terminating groups. Examples of de-blocking agents can include tetrabutylammonium fluoride, pyridine-HF, with ammonium fluoride, and triethylamine trihydrofluoride, for chain-terminating groups urea and silyl.

As used herein, the term “sequencing” and related terms refers to a method for obtaining nucleotide sequence information from a nucleic acid molecule, typically by determining the identity of at least some nucleotides (including their nucleobase components) within the nucleic acid molecule. In some embodiments, the sequence information of a given region of a nucleic acid molecule includes identifying each and every nucleotide within a region that is sequenced. In some embodiments, sequencing information determines only some of the nucleotides within a region, while the identity of some nucleotides remains undetermined or incorrectly determined. Any suitable method of sequencing known in the art may be used. In an exemplary embodiment, sequencing can include label-free methods. In another embodiment, sequencing can include ion-based sequencing methods. In some embodiments, sequencing can include labeled or dye-containing nucleotide or fluorescent based nucleotide sequencing methods. In some embodiments, sequencing can include polony-based sequencing or bridge sequencing methods. In some embodiments, the sequencing employs polymerases and multivalent molecules for generating at least one avidity complex, wherein individual multivalent molecules comprise a plurality of nucleotide units tethered to a core. In some embodiments, the sequencing employs polymerases and free nucleotides for performing sequencing-by-synthesis. In some embodiments, the sequencing employs a ligase enzyme and a plurality of sequence-specific oligonucleotides for performing sequence-by-ligation.

### INTRODUCTION

In some aspects, the present disclosure provides compositions and methods for sequencing different regions of the same nucleic acid template molecule and reducing residual signals that contribute to poor sequencing quality. The provided compositions and methods can be employed for pairwise sequencing workflows. The provided compositions and methods can be employed for uniplex and multiplex sequencing workflows.

Traditional multiplex sequencing workflows require sequencing a first region (e.g., a sequence-of-interest region) using a first sequencing primer, and sequencing a second region (e.g., a sample index region) using a second sequencing primer. The first and second sequencing primers are generally designed to hybridize to different regions of the same template molecule for conducting separate sequencing reactions to generate separate reads of the sequence-of-interest and sample index(es) regions. The sequencing read products are typically generated by conducting polymerase-catalyzed primer extension reactions. The sequencing read products that are synthesized from sequencing the first region must be efficiently removed prior to sequencing the second region while leaving the template molecule undamaged. Traditional sequencing workflows that employ chemical-based reagents (e.g., NaOH and/or formamide) to denature the sequencing read products can be harsh, resulting in damage to the template molecule. Switching to gentler chemical-based denaturation conditions may be less damaging to the template molecule, but do not effectively remove the sequencing read products, which contribute to residual signals. After multiple sequencing cycles, the residual signals can accumulate, making it difficult to image and detect true sequencing signals. Residual signals cause poor sequencing quality and reduce sequencing accuracy.

The order of sequencing the sequence-of-interest and the sample index(es) regions also presents a challenge. When the sequence-of-interest region is sequenced before the sample index(es), the longer sequencing read product of the sequence-of-interest region must be removed prior to sequencing the shorter sample index regions. Inefficient removal of the sequencing read product from the sequence-of-interest region in previous sequencing cycles will generate residual signals in subsequent sequencing cycles of the sample index region. Accumulation of residual signals in subsequent sequencing cycles reduces sequencing accuracy, which can lead to mis-alignment of sample indexes with their proper sequence-of-interest regions. This phenomenon is known as index hopping.

Thus, efficient removal of a sequencing read product from previous sequencing cycles will improve the overall accuracy of sequencing data. High accuracy sequencing of different regions of the template molecule is dependent on many parameters, including high signal intensity, reduced residual signals from previous sequencing reads, and preserving intact template molecules after multiple sequencing cycles.

In some aspects, the present disclosure provides compositions comprising enzyme-based reagents, and methods that employ the enzyme-based reagents, for efficiently removing sequencing read products prior to conducting subsequent sequencing cycles. The enzyme-based reagents offer several advantages over the traditional chemical-based reagents that use formamide, alkaline conditions and/or elevated temperatures for denaturing sequencing read products in a sequencing workflow. The enzyme-based reagents do not require formamide or alkaline conditions. The enzyme-based workflow can be conducted at low temperatures that pose reduced risk of damaging the template molecules.

It is well known that formamide lowers the melting temperature of duplex DNA in a linear manner by about 2.4-2.9° C. per mole of formamide, or about 0.6° C. per percent formamide. The length of the duplexed region and the guanine and cytosine (G+C) composition also influences the melting temperature of duplexed DNA. A common chemical-based method for removing sequencing read products includes denaturation at 50-75° C. in the presence of 20-40% formamide. Denaturation of duplex DNA at high temperatures can cause strand breakage and depurination, which can lead to loss of intact template molecules after multiple sequencing cycles, and reduced signal intensity. Additionally, formamide is classified as a reproductive toxic substance which can be inhaled, ingested or absorbed through the skin. Laboratory safety regulations dictate that disposal of any reagent containing formamide must be treated as a chemical waste.

By contrast, the enzyme-based reagents of the methods described herein can be used to efficiently remove sequencing read products using enzyme degradation conducted at a temperature considerably lower than a temperature used to denature double-stranded nucleic acids. For example, the enzyme degradation can be conducted at a temperature no higher than about 25-37° C., which is a temperature range that does not efficiently denature duplexed DNA using a formamide-based reagent. Using the enzyme-based reagents of the present disclosure at a lower temperature to remove the sequencing read products reduces damage to the template molecules and preserves intact template molecules after numerous sequencing cycles.

The enzyme-based reagents described herein degrade/remove a higher percentage of the sequencing read products from the template molecules compared to denaturing the sequencing read product using a chemical-based denaturing reagent (e.g., comprising formamide) at an elevated temperature (e.g., a temperature of about 50-70° C.). Thus, the enzyme-based reagents, and methods using the same, efficiently remove the sequencing read products and reduces residual signals from a previous sequencing cycle, while preserving intact template molecules over multiple sequencing cycles.

### Enzyme-Based Compositions

In some aspects, I present disclosure provides a composition comprising an enzyme complex which includes (a) a nucleic acid duplex immobilized to a support; and (b) an enzyme bound to the immobilized nucleic acid duplex. In some embodiments, the immobilized nucleic acid duplex comprises (i) a single-stranded nucleic acid template molecule which is immobilized to the support, and (ii) a sequencing read product which is hybridized to a portion of the immobilized single-stranded template molecule, where the sequencing read product comprises a sequencing primer (e.g., an oligonucleotide) joined to a polynucleotide having a sequence that is complementary to at least a portion of the single-stranded nucleic acid template molecule. In some embodiments, the enzyme comprises a double-stranded DNA specific exonuclease having 5′ to 3′ activity. In some embodiments, any portion of the single-stranded template molecule is immobilized to the support or immobilized to a coating on the support. For example, the 5′ or 3′ end of the template molecule, or an internal portion of the template molecule, is immobilized to the support. In some embodiments, the composition comprises a plurality of enzyme complexes immobilized to a support where each complex comprises: (a) a nucleic acid duplex immobilized to a support; and (b) at least one enzyme bound to the immobilized nucleic acid duplex.

In some embodiments, the at least one enzyme has 5′ to 3′ exonuclease activity. In some embodiments, the at least one enzyme has 3′ to 5′ exonuclease activity. In some embodiments, the at least one enzyme comprises a mixture of at least one enzyme having 5′ to 3′ exonuclease activity and at least one enzyme having 3′ to 5′ exonuclease activity.

In some embodiments, the at least one enzyme comprises an exonuclease that initiates degradation at the 5′ terminus of double-stranded DNA including linear and circular DNA molecules and nicked double-stranded DNA molecules. The exonuclease degrades double-stranded DNA in a 5′ to 3′ direction and can release mononucleotides and oligonucleotides. In some embodiments, the exonuclease lacks endonuclease activity. In some embodiments, the exonuclease comprises a T7 exonuclease encoded by T7 phage gene 6. T7 exonuclease gene 6 is commercially-available from New England Biolabs® (e.g., catalog No. M0263S) and Thermo Fisher Scientific® (e.g., catalog No. 70025Z10KU). In some embodiments, the exonuclease comprises a lambda exonuclease. In some embodiments, the lambda exonuclease can degrade the 5′ phosphorylated end of one strand of double-stranded DNA. In some embodiments, the lambda exonuclease is from New England Biolabs® (e.g., catalog No. M0262S).

In some embodiments, the at least one enzyme comprises an exonuclease that initiates degradation at the 3′ terminus of double-stranded DNA including linear and circular DNA molecules and nicked double-stranded DNA molecules. The exonuclease can degrade double-stranded DNA in a 3′ to 5′ direction and can release mononucleotides and/or oligonucleotides. In some embodiments, the exonuclease lacks endonuclease activity. In some embodiments, the exonuclease comprises E. coli exonuclease III (e.g., catalog No. M0206S from New England Biolabs®) or E. coli exonuclease I (e.g., catalog No. M0568 from New England Biolabs®).

In some embodiments, it is advantageous to employ an exonuclease that degrades double-stranded DNA in a 5′ to 3′ direction when the template molecules to be sequenced comprise single-stranded linear DNA template molecules that are immobilized to a support by their 5′ ends and having their 3′ ends free, and the sequencing read products each comprise a sequencing primer which is annealed to a portion of a given template molecule and subjected to a polymerase-catalyzed primer extension reaction. A 5′ to 3′ exonuclease enzyme, such as T7 exonuclease or lambda exonuclease, can be removed from the template molecule with a washing buffer at a temperature of about 22-25° C., or about 25-30° C., without the need for heat deactivation. By contrast, heat deactivation can be required to inactive certain exonucleases having 3′ to 5′ exonuclease activity. For example, E. coli exonuclease III must be heat deactivated at 70° C., and E. coli exonuclease I must be heat deactivated at 80° C. When DNA molecules are subjected to a pairwise sequencing workflow, e.g., where a first region is sequenced and the first sequencing read product is removed prior to sequencing a second region, the DNA template molecules can be damaged when subjected to enzyme deactivation conditions using high heat.

In some embodiments, the enzyme is formulated in an enzyme-based reagent comprising a double-stranded DNA specific exonuclease, at least one aqueous solvent, a pH buffering agent, a monovalent salt, a divalent salt, and enzyme stabilizer (e.g., BSA). In some embodiments, the aqueous solvent comprises water. In some embodiments, the monovalent salt comprises potassium, for example potassium acetate. In some embodiments, the divalent salt comprises magnesium, for example magnesium acetate. In some embodiments, the enzyme stabilizer comprises a protein comprising bovine serum albumin (BSA), beta-lactoglobulin (e.g., from bovine milk), beta-casein (e.g., from bovine milk) or ovalbumin. In some embodiments, the enzyme-bases reagent has a pH of about 6-9, or about 7-9. In some embodiments, the enzyme-based reagent further comprises NaCl at a concentration suitable to reduce 5′ flap endonuclease activity of the T7 exonuclease. The enzyme-based reagent can include NaCl at a concentration of about 20-150 mM, or about 20-40 mM, or about 40-60 mM, or about 60-80 mM, or about 80-100 mM, or about 100-120 mM, or about 120-150 mM, or any range therebetween.

In some embodiments, the 5′ or 3′ end of the single-stranded nucleic acid template molecule is covalently attached to a surface primer which is immobilized to the support or immobilized to a coating on the support. In some embodiments, the 5′ or 3′ region of the single-stranded nucleic acid template molecule is hybridized to a surface primer which is immobilized to the support or immobilized to a coating on the support.

In some embodiments, the single-stranded nucleic acid template molecule can be generated by a clonal amplification workflow. In some embodiments, the single-stranded nucleic acid template molecule was not generated by a clonal amplification workflow.

In some embodiments, the single-stranded nucleic acid template molecule includes at least one uridine nucleotide. In some embodiments, the single-stranded nucleic acid template molecule lacks a uridine nucleotide.

In some embodiments, the single-stranded nucleic acid template molecule comprises one copy of the sequence-of-interest. For example, the one-copy template molecule can be generated by bridge amplification. In some embodiments, the single-stranded nucleic acid template molecule comprises two or more tandem copies of the sequence-of-interest. For example, and without limitation, the tandem-copy template molecule can comprise a concatemer which can be generated by rolling circle amplification (RCA).

In some embodiments, the single-stranded nucleic acid template molecule comprises at least one sequence-of-interest (e.g., insert region). In some embodiments, the single-stranded nucleic acid template molecule further comprises at least one universal adaptor sequence including any one or any combination of: a first surface primer binding site (e.g., capture primer binding site); a second surface primer binding site (e.g., surface pinning primer binding site); a first sequencing primer binding site (e.g., forward sequencing primer binding site); a second sequencing primer binding site (e.g., reverse sequencing primer binding site); a first amplification primer binding site (e.g., forward amplification primer binding site); a second amplification primer binding site (e.g., reverse amplification primer binding site); a first sample index sequence; a second sample index sequence; a first unique molecular tag sequence; a second unique molecular tag sequence; a first compaction oligonucleotide binding site; and/or a second compaction oligonucleotide binding site.

In some embodiments, the first surface primer binding site (or a complementary sequence thereof) can hybridize to at least a portion of the immobilized first surface primer. In some embodiments, the second surface primer binding site (or a complementary sequence thereof) can hybridize to at least a portion of the immobilized second surface primer. In some embodiments, the first sequencing primer site (or a complementary sequence thereof) can hybridize to at least a portion of the forward sequencing primer. In some embodiments, the second sequencing primer site (or a complementary sequence thereof) can hybridize to at least a portion of the reverse sequencing primer. In some embodiments, the first amplification primer binding site (or a complementary sequence thereof) can hybridize to at least a portion of a forward amplification primer. In some embodiments, the second amplification primer binding site (or a complementary sequence thereof) can hybridize to at least a portion of a reverse amplification primer. In some embodiments, the first compaction oligonucleotide binding site (or a complementary sequence thereof) can hybridize to at least a portion of a first compaction oligonucleotide. In some embodiments, the second compaction oligonucleotide binding site (or a complementary sequence thereof) can hybridize to at least a portion of a second compaction oligonucleotide.

In some embodiments, the sequencing read products comprise a sequencing primer (e.g., a nucleic acid oligonucleotide) which is joined to a polynucleotide having a sequence that is complementary to at least a portion of the single-stranded nucleic acid template molecule. At least a portion of the complementary polynucleotide can be generated by any type of sequencing workflow comprising a polymerase-catalyzed primer extension reaction in which the sequence of the single-strand template molecule is determined. At least a portion of the complementary polynucleotide can be generated by any type of polymerase-catalyzed primer extension reaction in which the sequence of the single-strand template molecule is not determined. In some embodiments, the sequencing primer portion of the sequencing read product has an extendible 3′ terminal end or a non-extendible 3′ terminal end.

In some embodiments, the support comprises a planar or non-planar support. The support can be solid or semi-solid. In some embodiments, the support can be porous, semi-porous or non-porous. The support can be made of any material such as glass, plastic or a polymer material.

In some embodiments, the surface of the support can be coated with one or more compounds to produce a passivated layer on the support. In some embodiments, the passivated layer forms a porous or semi-porous layer. In some embodiments, the surface primer of the single-stranded nucleic template molecule can be attached to the passivated layer to immobilize the primer or template molecule to the support. In some embodiments, the support comprises a low non-specific binding surface that enables improved nucleic acid hybridization, amplification and sequencing performance on the support. In some embodiments, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid template molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacet-amidylpentyl)acrylamide-co-acrylamide (PAZAM). In some embodiments, the support comprises a surface coating having at least one hydrophilic polymer coating layer and at least one layer of a plurality of oligonucleotides. The hydrophilic polymer coating layer can comprise polyethylene glycol (PEG). The hydrophilic polymer coating layer can comprise branched PEG having at least 4 branches. In some embodiments, the low non-specific binding coating has a degree of hydrophilicity which can be measured as a water contact angle, where the water contact angle is no more than 45 degrees (e.g., no more than 5 degrees, no more than 10 degrees, no more than 15 degrees, no more than 20 degrees, no more than 25 degrees, no more than 30 degrees, no more than 35 degrees, no more than 40 degrees, or no more than 45 degrees).

In some embodiments, the support comprises a plurality of single-stranded nucleic acid template molecules immobilized to the support or immobilized to a coating on the support. In some embodiments, about 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) template molecules are immobilized to the support at different sites on the support. In some embodiments, the plurality of template molecule is immobilized to pre-determined sites (e.g., locations) on the support. In some embodiments, the plurality of template molecules is immobilized to random sites (e.g., locations) on the support. In some embodiments, the plurality of immobilized template molecules is in fluid communication with each other to permit flowing a solution of reagents (e.g., enzymes including polymerases, multivalent molecules, nucleotides and/or divalent cations, and the like) onto the support so that the plurality of immobilized template molecules on the support can be reacted with the solution of reagents in a massively parallel manner.

### Methods Using Enzyme-Based Reagents—Sequencing a Template Strand

In some aspects, the present disclosure provides a method for removing a sequencing read product from a nucleic acid template molecule by degradation using an enzyme-based reagent. The methods can be used for sequencing one or more regions of the same template molecule.

In some aspects, the present disclosure provide a method for sequencing a nucleic acid template molecule, comprising (a): providing at least one single-stranded nucleic acid template molecule immobilized to a support; (b) hybridizing at least one first sequencing primer to a first region of the nucleic acid template molecule and conducting one or more sequencing reactions (e.g., conducting one or more sequencing cycle reactions) thereby generating a first extension duplex comprising a first sequencing read product hybridized to the template molecule; and (c) contacting the first extension duplex with a double-stranded DNA specific exonuclease under a condition suitable to degrade the first sequencing read product and retain the template molecule thereby removing at least a portion of the first sequencing read product from the first region of the template molecule. In some embodiments, the method further comprises step (d): washing the retained template molecule at a temperature of 22-25° C. or 25-30° C. to remove the double-stranded DNA specific enzyme.

In some embodiments, the methods further comprise: (e) hybridizing at least one second sequencing primer to a second region of the same nucleic acid template molecule and conducting one or more sequencing reactions (e.g., conducting one or more sequencing cycle reactions) thereby generating a second extension duplex comprising a second sequencing read product hybridized to the template molecule; and (f) contacting the second extension duplex with a double-stranded DNA specific exonuclease under a condition suitable to degrade the second sequencing read product and retain the template molecule thereby removing at least a portion of the second sequencing read product from the second region of the template molecule. In some embodiments, the method further comprises step (g): washing the retained template molecule at a temperature of 22-25° C. or 25-30° C. to remove the double-stranded DNA specific enzyme.

In some embodiments, the first and second sequencing primers hybridize to different and non-overlapping regions of the same nucleic acid template molecule. In some embodiments, the first and second sequencing primers hybridize to the same region of the same nucleic acid template molecule. In some embodiments, the first and second sequencing primers hybridize to overlapping regions of the same nucleic acid template molecule.

In some embodiments, the methods further comprise: (h) hybridizing at least one third sequencing primer to a third region of the same nucleic acid template molecule and conducting one or more sequencing reactions (e.g., conducting one or more sequencing cycle reactions) thereby generating a third extension duplex comprising a third sequencing read product hybridized to the template molecule; and (i) contacting the third extension duplex with a double-stranded DNA specific exonuclease under a condition suitable to degrade the third sequencing read product and retain the template molecule thereby removing at least a portion of the third sequencing read product from the third region of the template molecule.

In some embodiments, the method further comprises step (j): washing the retained template molecule at a temperature of 22-25° C. or 25-30° C. to remove the double-stranded DNA specific enzyme.

In some embodiments, the second and third sequencing primers hybridize to different and non-overlapping regions of the same nucleic acid template molecule. In some embodiments, the second and third sequencing primers hybridize to the same region of the same nucleic acid template molecule. In some embodiments, the second and third sequencing primers hybridize to overlapping regions of the same nucleic acid template molecule.

In some embodiments, the method further comprises repeating steps (h)-(j) using a fourth sequencing primer that hybridizes to a fourth region of the same template molecule. The skilled artisan recognizes that steps (h)-(j) can be repeated multiple times using a fourth, fifth, sixth, seventh, eighth, ninth, tenth or more sequencing primers that hybridize to their respective regions of the same template molecule.

In some embodiments, the third and fourth sequencing primers hybridize to different and non-overlapping regions of the same nucleic acid template molecule. In some embodiments, the third and fourth sequencing primers hybridize to the same region of the same nucleic acid template molecule. In some embodiments, the third and fourth sequencing primers hybridize to overlapping regions of the same nucleic acid template molecule.

### Methods Using Enzyme-Based Reagents—Sequencing a Plurality of Template Strands

In some aspects, the present disclosure provides a method for removing a plurality of sequencing read products from a plurality of nucleic acid template molecules by degradation using an enzyme-based reagent. The methods can be used for sequencing one or more regions of the same template molecules.

In some embodiments, the method for sequencing a plurality of nucleic acid template molecules comprise: (a): providing a plurality of single-stranded nucleic acid template molecules immobilized to a support; (b) hybridizing at least one first sequencing primer to a first region of individual nucleic acid template molecules in the plurality of template molecules and conducting one or more sequencing reactions (e.g., conducting one or more sequencing cycle reactions) thereby generating a plurality of first extension duplexes each comprising a first sequencing read product hybridized to a template molecule; and (c) contacting the first extension duplexes with a double-stranded DNA specific exonuclease under a condition suitable to degrade the first sequencing read products and retain the template molecules thereby removing at least a portion of the first sequencing read products from the first region of the template molecules. In some embodiments, the method further comprises step (d): washing the plurality of retained template molecules at a temperature of 22-25° C. or 25-30° C. to remove the double-stranded DNA specific enzymes.

In some embodiments, the methods further comprise: (e) hybridizing at least one second sequencing primer to a second region of individual nucleic acid template molecules in the plurality of template molecules and conducting one or more sequencing reactions (e.g., conducting one or more sequencing cycle reactions) thereby generating a plurality of second extension duplexes each comprising a second sequencing read product hybridized to a template molecule; and (f) contacting the plurality of second extension duplexes with a double-stranded DNA specific exonuclease under a condition suitable to degrade the second sequencing read products and retain the template molecules thereby removing at least a portion of the second sequencing read products from the second region of the template molecules. In some embodiments, the method further comprises step (g): washing the plurality of retained template molecules at a temperature of 22-25° C. or 25-30° C. to remove the double-stranded DNA specific enzymes.

In some embodiments, the first and second sequencing primers hybridize to different and non-overlapping regions of the same nucleic acid template molecule. In some embodiments, the first and second sequencing primers hybridize to the same region of the same nucleic acid template molecule. In some embodiments, the first and second sequencing primers hybridize to overlapping regions of the same nucleic acid template molecule.

In some embodiments, the methods further comprise: (h) hybridizing at least one third sequencing primer to a third region of individual nucleic acid template molecules in the plurality of template molecules and conducting one or more sequencing reactions (e.g., conducting one or more sequencing cycle reactions) thereby generating a plurality of third extension duplexes each comprising a third sequencing read product hybridized to a template molecule; and (i) contacting the plurality of third extension duplexes with a double-stranded DNA specific exonuclease under a condition suitable to degrade the third sequencing read products and retain the template molecules thereby removing at least a portion of the third sequencing read products from the third region of the template molecules. In some embodiments, the method further comprises step (j): washing the plurality of retained template molecules at a temperature of 22-25° C. or 25-30° C. to remove the double-stranded DNA specific enzymes.

In some embodiments, the second and third sequencing primers hybridize to different and non-overlapping regions of the same nucleic acid template molecule. In some embodiments, the second and third sequencing primers hybridize to the same region of the same nucleic acid template molecule. In some embodiments, the second and third sequencing primers hybridize to overlapping regions of the same nucleic acid template molecule.

In some embodiments, the method further comprises repeating steps (h)-(j) using a fourth sequencing primer that hybridizes to a fourth region of individual template molecules in the plurality of template molecules. The skilled artisan recognizes that steps (h) and (j) can be repeated multiple times using a fourth, fifth, sixth, seventh, eighth, nineth, tenth or more sequencing primers that hybridizes to their respective regions of individual template molecules.

In some embodiments, the third and fourth sequencing primers hybridize to different and non-overlapping regions of the same nucleic acid template molecule. In some embodiments, the third and fourth sequencing primers hybridize to the same region of the same nucleic acid template molecule. In some embodiments, the third and fourth sequencing primers hybridize to overlapping regions of the same nucleic acid template molecule.

### Methods for Pairwise Sequencing

In some aspects, the present disclosure provide methods for pairwise sequencing which comprises obtaining a first sequencing read of a first region of a first nucleic acid strand (e.g., sense strand or forward strand), and obtaining a second sequencing read of a second region of a second nucleic acid strand that is complementary to the first stand (e.g. anti-sense strand or reverse strand), wherein the first and second strands correspond to two complementary strands of the same double stranded template molecule. The first sequencing read of the first sequenced region and the second sequencing read of the second sequenced region can having overlapping sequences which correspond to complementary sequences from the first and second strands of the double stranded template molecule. The first and second sequencing reads can be aligned so that the overlapping sequencing reads can yield sequence information of a paired region in the original double stranded nucleic acid source (e.g., a paired region in the genome), and the accuracy of the sequence information can be ascertained from the first and second sequencing reads with a high level of confidence. The first sequencing read of the first sequenced region and the second sequencing read of the second sequenced region do not necessarily have overlapping sequences, in which case sequence information of a paired region in the original double stranded nucleic acid source cannot be ascertained with a high level of confidence. The first and second sequencing reads can initiate at one end of their respective template molecules, or can initiate at an internal position.

In some aspects, the present disclosure provides pairwise sequencing methods, comprising step (a): providing a plurality of immobilized single stranded nucleic acid template molecules each comprising at least one nucleotide having a scissile moiety, wherein individual template molecules in the plurality are immobilized to a first surface primer that is immobilized to a support, and wherein the immobilized first surface primer lacks a nucleotide having a scissile moiety. In some embodiments, the support comprises a plurality of first surface primers. In some embodiments, the support lacks a plurality of second surface primers. In some embodiments, the support comprises a plurality of first and second surface primers.

In some embodiments, in the methods for pairwise sequencing described herein, individual immobilized template molecules comprise concatemer molecules which include multiple tandem copies of the sequence-of-interest and at least one universal adaptor sequence (e.g., a sequencing primer binding sequence). In some embodiments, individual immobilized template molecules are non-concatemer molecules which comprise one copy of the sequence-of-interest and at least one universal adaptor sequence (e.g., a sequencing primer binding sequence).

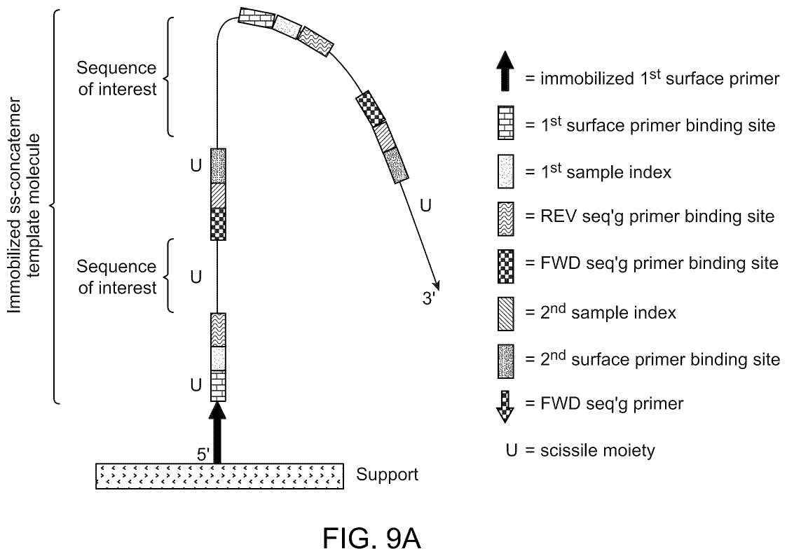

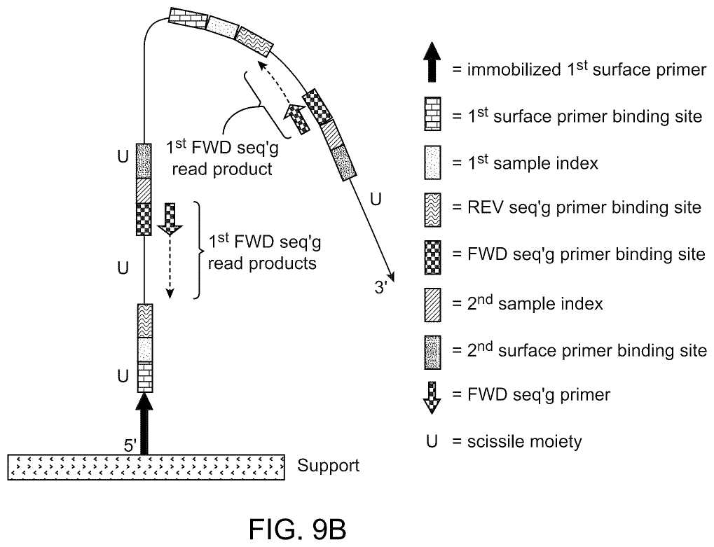

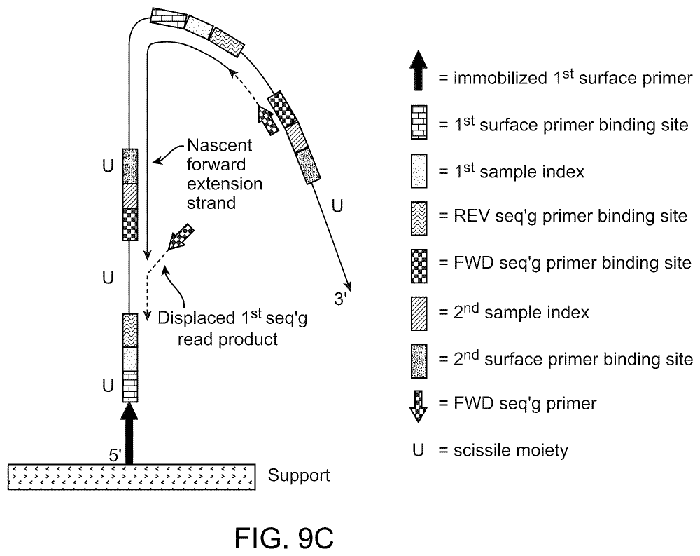

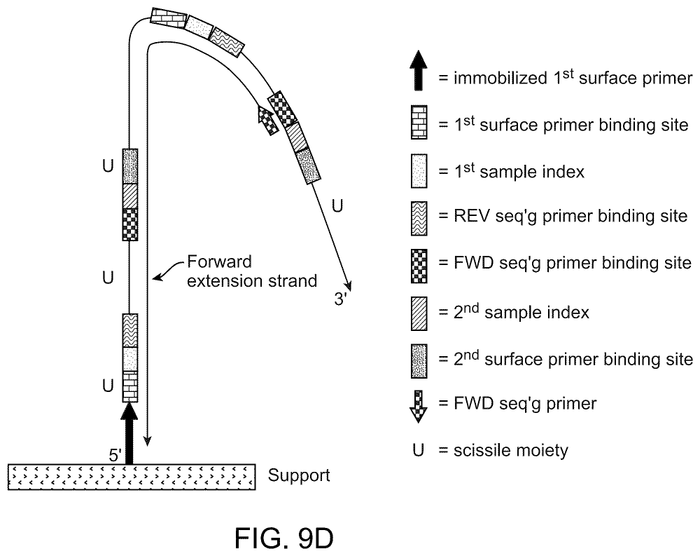

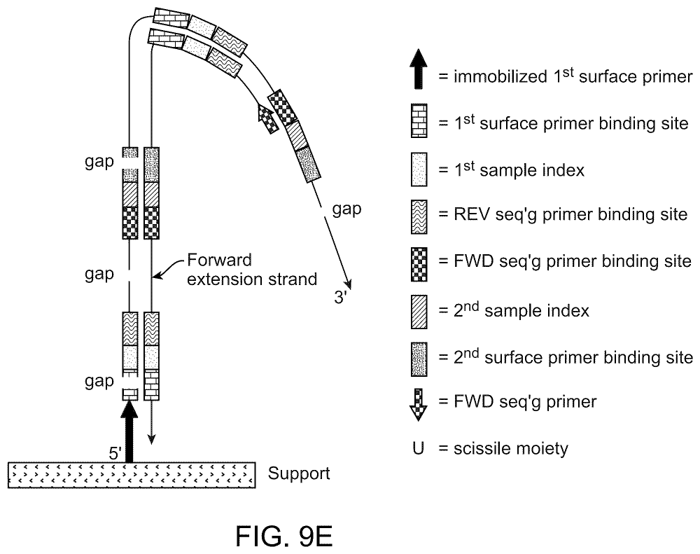

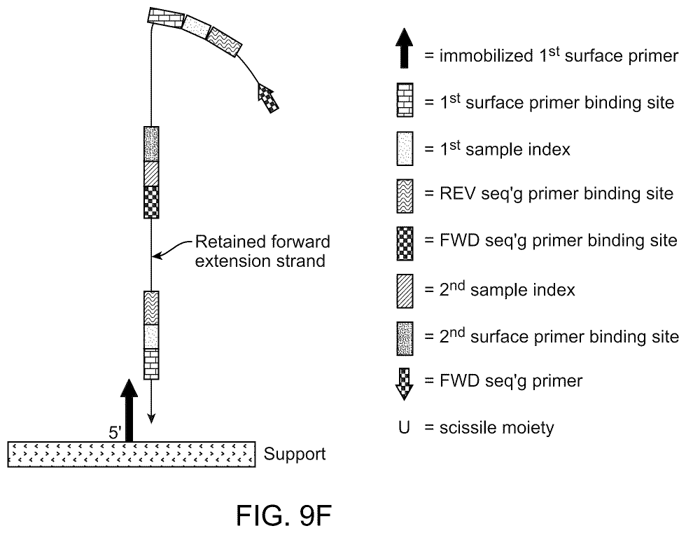

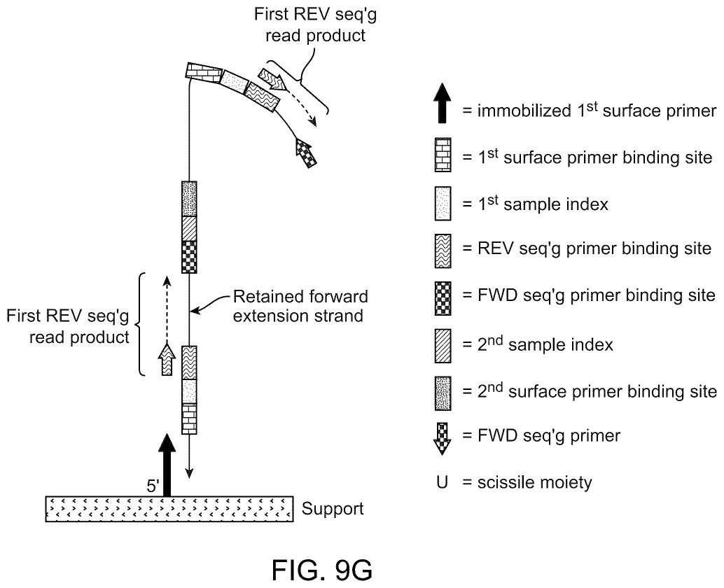

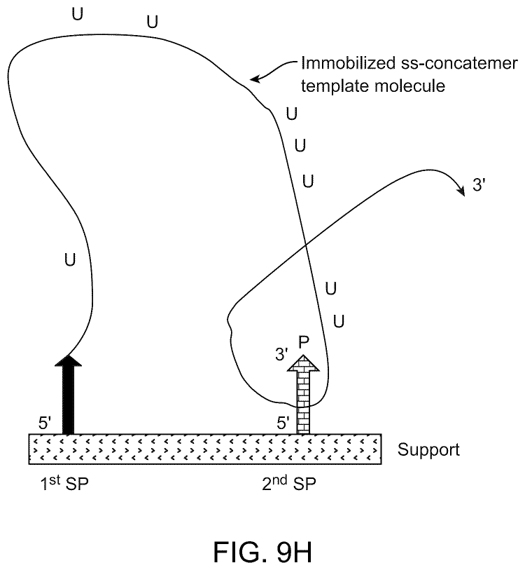

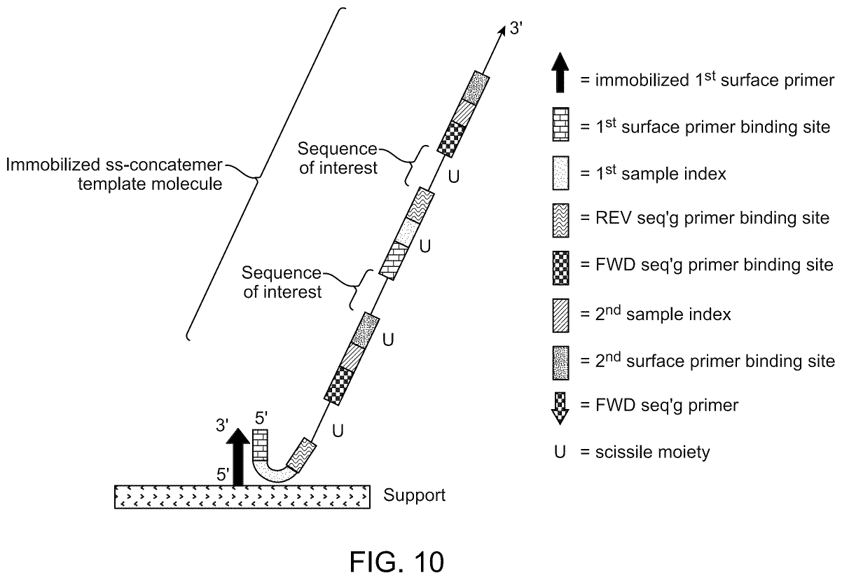

In some embodiments, in the methods for pairwise sequencing, individual immobilized template molecules are covalently joined to an immobilized surface primer (e.g., an immobilized first surface primer) (FIG. 9A). In an alternative embodiment, individual immobilized template molecules are hybridized to an immobilized surface primer (e.g., an immobilized first surface primer) (FIG. 10).

In some embodiments, in the methods for pairwise sequencing, individual template molecules in the plurality comprise at least one sequence-of-interest and at least one universal adaptor sequence or any combination of two or more of universal adaptor sequences including: a first surface primer binding site (or a complementary sequence thereof) which can hybridize to at least a portion of the immobilized first surface primer; a second surface primer binding site (or a complementary sequence thereof) which can hybridize to at least a portion of the immobilized second surface primer; a first sequencing primer site (or a complementary sequence thereof) which can hybridize to at least a portion of the forward sequencing primer; a second sequencing primer site (or a complementary sequence thereof) which can hybridize to at least a portion of the reverse sequencing primer; a first amplification primer binding site (or a complementary sequence thereof) which can hybridize to at least a portion of a forward amplification primer; a second amplification primer binding site (or a complementary sequence thereof) which can hybridize to at least a portion of a reverse amplification primer; a first compaction oligonucleotide binding site (or a complementary sequence thereof) which can hybridize to at least a portion of a first compaction oligonucleotide; a second compaction oligonucleotide binding site (or a complementary sequence thereof) which can hybridize to at least a portion of a second compaction oligonucleotide; a first sample index sequence; a second sample index sequence; a first unique molecular tag sequence; and/or a second unique molecular tag sequence.

In some embodiments, in the methods for pairwise sequencing, the scissile moiety in the immobilized template molecules of step (a) can be converted into abasic sites in the immobilized template molecules. In some embodiments, the scissile moiety in the immobilized template molecules comprises uridine, 8-oxo-7,8-dihydroguanine (e.g., 8oxoG) or deoxyinosine. In the template molecules, the uridine can be converted to an abasic site using uracil DNA glycosylase (UDG), the 8oxoG can be converted to an abasic site using FPG glycosylase, and the deoxyinosine can be converted to an abasic site using AlkA glycosylase. In some embodiments, the immobilized template molecules include 1-20, 20-40, 40-60, 60-80, 80-100, any range therebetween, or a higher number of nucleotides with a scissile moiety. In some embodiments, about 0.1-1%, or about 1-5%, or about 5-10%, or about 10-20%, or about 20-30%, any range therebetween, or a higher percent of the dTTP in the immobilized template molecules are replaced with nucleotides having a scissile moiety. In some embodiments, the nucleotides having a scissile moiety are distributed at random positions along individual immobilized template molecules. In some embodiments, the nucleotides having a scissile moiety are distributed at different positions in the different immobilized template molecules.

In some embodiments, in the methods for pairwise sequencing, the immobilized first surface primers comprise single stranded oligonucleotides comprising DNA, RNA or a combination of DNA and RNA. The immobilized first surface primers can be immobilized to the support or immobilized to a coating on the support. The immobilized first surface primers can be embedded and attached (coupled) to the coating on the support. In some embodiments, the 5′ end of the immobilized first surface primers are immobilized to a support or immobilized to a coating on the support. Alternatively, an interior portion or the 3′ end of the immobilized first surface primers can be immobilized to a support or immobilized to a coating on the support. The support can comprise a plurality of immobilized first surface primers having the same sequence. The immobilized first surface primers can be any length, for example 4-50 nucleotides, or 50-100 nucleotides, or 100-150 nucleotides, any range therebetween, or longer lengths. In some embodiments, the 3′ terminal end of the immobilized first surface primers comprise an extendible 3′ OH moiety. In some embodiments, the 3′ terminal end of the immobilized first surface primers comprise a 3′ non-extendible moiety. The immobilized first surface primers lack a nucleotide having a scissile moiety.

In some embodiments, in the methods for pairwise sequencing, the plurality of immobilized first surface primers comprise at least one phosphorothioate diester bond at their 5′ ends which can render the first surface primers resistant to exonuclease degradation. In some embodiments, the plurality of immobilized first surface primers comprise 2-5 or more consecutive phosphorothioate diester bonds at their 5′ ends. In some embodiments, the plurality of immobilized first surface primers comprise at least one ribonucleotide and/or at least one 2′-O-methyl or 2′-O-methoxyethyl (MOE) nucleotide which can render the first surface primers resistant to exonuclease degradation.

In some embodiments, in the methods for pairwise sequencing, the immobilized first surface primers comprise at least one locked nucleic acid (LNA) which comprises a methylene bridge bond between a 2′ oxygen and 4′ carbon of the pentose ring. Immobilized first surface primers that include at least one LNA can be resistant to nuclease digestions and can exhibit increased melting temperature when hybridized to the forward extension strand.

In some embodiments, in the methods for pairwise sequencing, the immobilized template molecules further comprise two or more copies of a universal binding sequence (or complementary sequence thereof) for an immobilized second surface primer having a sequence that differs from the first immobilized surface primer. The immobilized second surface primers of step (a) can comprise single stranded oligonucleotides comprising DNA, RNA or a combination of DNA and RNA. The immobilized second surface primers can be immobilized to the support or immobilized to a coating on the support. The immobilized second surface primers can be embedded and attached (coupled) to the coating on the support. In some embodiments, the 5′ end of the immobilized second surface primers are immobilized to a support or immobilized to a coating on the support. Alternatively, an interior portion or the 3′ end of the immobilized second surface primers can be immobilized to a support or immobilized to a coating on the support. The support can comprise a plurality of immobilized second surface primers having the same sequence. The immobilized second surface primers can be any length, for example 4-50 nucleotides, or 50-100 nucleotides, or 100-150 nucleotides, any range therebetween, or longer lengths.

In some embodiments, in the methods for pairwise sequencing, the 3′ terminal end of the immobilized second surface primers comprise an extendible 3′ OH moiety. In some embodiments, the 3′ terminal end of the immobilized second surface primers comprise a 3′ non-extendible moiety. In some embodiments, the 3′ terminal end of the immobilized second surface primers comprise a moiety that blocks primer extension (e.g., non-extendible terminal 3′ end), such as for example a phosphate group, a dideoxycytidine group, an inverted dT, or an amino group. The immobilized second surface primers are not extendible in a primer extension reaction. The immobilized second surface primers lack a nucleotide having a scissile moiety.

In some embodiments, in the methods for pairwise sequencing, the plurality of immobilized second surface primers comprise at least one phosphorothioate diester bond at their 5′ ends which can render the second surface primers resistant to exonuclease degradation. In some embodiments, the plurality of immobilized second surface primers comprise 2-5, or more, consecutive phosphorothioate diester bonds at their 5′ ends. In some embodiments, the plurality of immobilized second surface primers comprise at least one ribonucleotide and/or at least one 2′-O-methyl or 2′-O-methoxyethyl (MOE) nucleotide which can render the second surface primers resistant to exonuclease degradation.

In some embodiments, in the methods for pairwise sequencing, individual immobilized single stranded nucleic acid template molecule are joined or immobilized to an immobilized first surface primer, and at least one portion of the individual template molecule is hybridized to an immobilized second surface primer. The immobilized second surface primers can serve to pin down a portion of the immobilized template molecules to the support (see FIG. 9H).

In some embodiments, in the methods for pairwise sequencing, the support comprises about 10²-10¹⁵ (e.g., 10², 10³, 10⁴, 10¹, 10⁶, 10⁷, 10⁸, 10⁹, 10¹⁰, 10¹¹, 10¹², 10¹³ 10¹⁴, or 10¹⁵) immobilized first surface primers per mm². In some embodiments, the support comprises about 10²-10¹⁵ (e.g., 10², 10³, 10⁴, 10¹, 10⁶, 10⁷, 10⁸, 10⁹, 10¹⁰, 10¹¹, 10¹², 10¹³ 10¹⁴, or 10¹⁵) immobilized second surface primers per mm². In some embodiments, the support comprises about 10²-10¹⁵ (e.g., 10², 10³, 10⁴, 10¹, 10⁶, 10⁷, 10⁸, 10⁹, 10¹⁰, 10¹¹, 10¹², 10¹³, 10¹⁴, or 10¹⁵) immobilized first surface primers and immobilized second surface primers per mm².

In some embodiments, in the methods for pairwise sequencing, the immobilized surface primers (e.g., first and second surface primers) are in fluid communication with each other to permit flowing various solutions of linear or circular nucleic acid template molecules, soluble primers, enzymes, nucleotides, divalent cations, buffers, reagents, and the like, onto the support so that the plurality of immobilized surface primers (and the primer extension products generated from the immobilized surface primers) react with the solutions in a massively parallel manner.

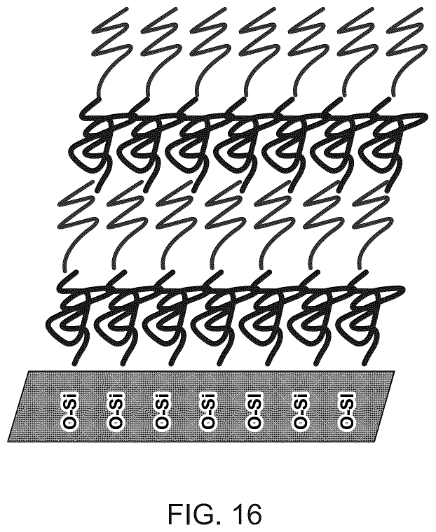

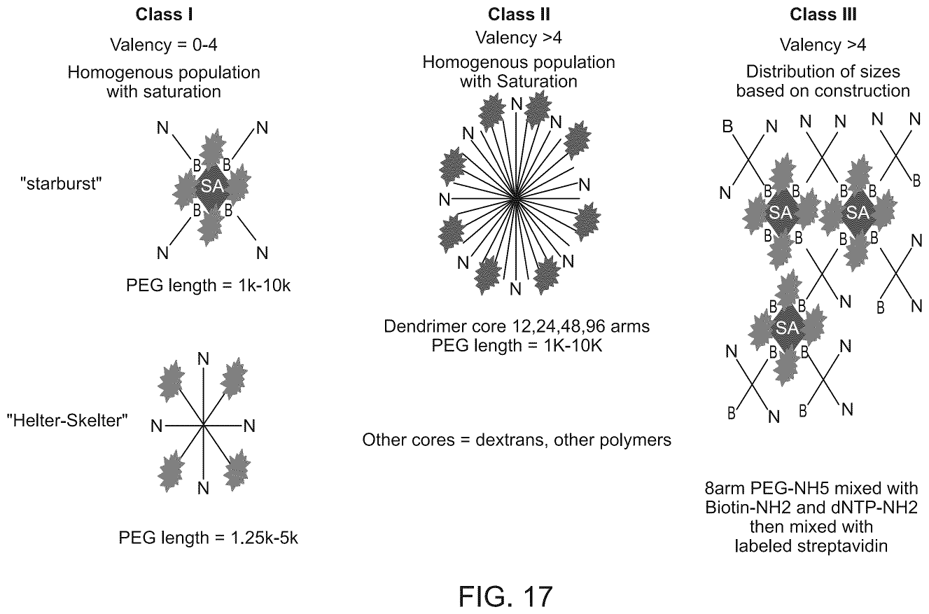

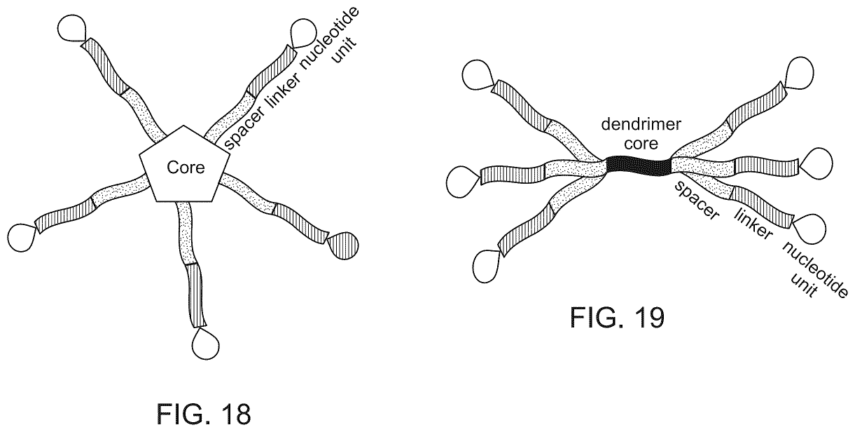

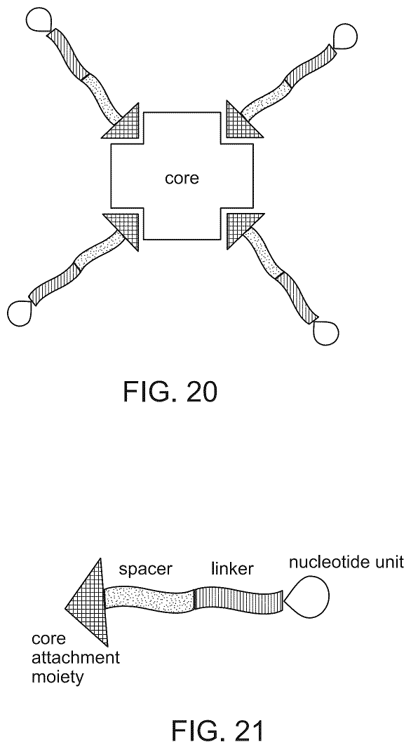

In some embodiments, the pairwise sequencing method further comprises step (b): sequencing the plurality of immobilized template molecules thereby generating a plurality of forward extension duplexes. The sequencing of step (b) comprises contacting the plurality of immobilized template molecules with a plurality of soluble forward sequencing primers under a condition suitable to hybridize at least one forward sequencing primer to at least one of the forward sequencing primer binding sites/sequences of the immobilized template molecules, and conducting forward sequencing reactions using one or more types of sequencing polymerases, a plurality of nucleotides and/or multivalent molecules, and the hybridized first forward sequencing primers. The forward sequencing reactions can generate a plurality of forward extension duplexes each comprising a first forward sequencing read product hybridized to an immobilized template molecule. Individual sequence read products comprise a forward sequencing primer joined to a polynucleotide having a sequence that is complementary to at least a portion of the immobilized template molecule. Individual forward sequencing primer binding sites in a given immobilized template molecule can be hybridized to a forward sequencing primer and can undergo a sequencing reaction. In some embodiments, individual immobilized concatemer template molecules have multiple copies of the forward sequencing primer binding sites, wherein each forward sequencing primer binding site is capable of hybridizing to a first forward sequencing primer. Individual immobilized concatemer template molecules can undergo two or more sequence reactions, where each sequencing reaction is initiated from a first forward sequencing primer that is hybridized to a forward sequencing primer binding site (e.g., see FIG. 9B). In some embodiments, the soluble forward sequencing primers comprise 3′ OH extendible ends. In some embodiments, the soluble forward sequencing primers comprise a 3′ blocking moiety which can be removed to generate a 3′ OH extendible end. In some embodiments, the soluble forward sequencing primers lack a nucleotide having a scissile moiety. In some embodiments, the sequencing reactions comprise a plurality of nucleotides (or analogs thereof) labeled with a detectable reporter moiety. In some embodiments, the sequencing reaction comprise a plurality of multivalent molecules having a plurality of nucleotide units attached to a core, where the multivalent molecules are labeled with a detectable reporter moiety. In some embodiments, the core is labeled with a detectable reporter moiety. In some embodiments, at least one linker and/or at least one nucleotide unit of a nucleotide arm is labeled with a detectable reporter moiety. In some embodiments, the detectable reporter moiety comprises a fluorophore. An exemplary nucleotide arm is shown in FIG. 21, and exemplary multivalent molecules are shown in FIGS. 17-20.

In some embodiments, the pairwise sequencing method further comprises step (c): retaining the plurality of immobilized template molecules and replacing the plurality of first forward sequencing read products with a plurality of forward extension strands that are hybridized to the retained immobilized single stranded nucleic acid template molecules. The plurality of first forward sequencing read products can be removed and replaced with a plurality of forward extension strands by conducting a primer extension reaction (see FIGS. 9C and 9D). The strand replacement step is sometimes referred to as “second strand synthesis” or “pairwise turn”. Described below are different embodiments for replacing the first forward sequencing read products by conducting primer extension reactions.

In some embodiments, in the methods for the pairwise sequencing, step (c) comprises contacting at least one first forward sequencing read product with a plurality of strand displacing polymerases and a plurality of nucleotides and in the absence of soluble amplification primers, under a condition suitable to conduct a strand displacing primer extension reaction using the at least one first forward sequencing read product to initiate the primer extension reaction thereby generating a nascent forward extension strand that is covalently joined to the first forward sequencing read product, wherein the forward extension strand is hybridized to the immobilized template molecule. For example, and without limitation, the 3′ end of a first forward sequencing read product can serve as a primer for the strand displacing polymerase. The strand displacing polymerase can extend the first forward sequencing read product, and displace downstream first forward sequencing read products while synthesizing a nascent forward extended strand that replaces the downstream first forward sequencing read products (FIG. 9C). The newly extended nascent strand is thus covalently joined to a first forward sequencing read product. The immobilized template molecules are retained.

In some embodiments, in the methods for the pairwise sequencing, the primer extension reaction of step (c) can optionally include a plurality of compaction oligonucleotides and/or hexamine (e.g., cobalt hexamine III) to generate the forward extension strands. Individual forward extension strands can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 μM or smaller.

Examples of strand displacing polymerases include, for example and without limitation, phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent DNA polymerase and KOD DNA polymerase. The phi29 DNA polymerase can be wild type phi29 DNA polymerase (e.g., MagniPhi™ from Expedeon®), or variant EquiPhi29 DNA polymerase (e.g., from Thermo Fisher Scientific®), or chimeric QualiPhi® DNA polymerase (e.g., from 4basebio®).

In some embodiments, in the methods for the pairwise sequencing, step (c) comprises: (i) removing the plurality of first sequence read products while retaining the immobilized template molecules; and (ii) contacting the plurality of retained immobilized molecules with a plurality of soluble forward sequencing primers (e.g., a second plurality of soluble forward sequencing primers), a plurality of nucleotides (e.g., a second plurality of nucleotides) and a plurality of primer extension polymerases, under a condition suitable to hybridize the plurality of soluble forward sequencing primers to the plurality of retained immobilized template molecules and suitable for conducting polymerase-catalyzed primer extension reactions thereby generating a plurality of forward extension strands, wherein the soluble sequencing primers hybridize with the forward sequencing primer binding sequence in the retained immobilized molecules (FIG. 9D). The primer extension reaction can optionally include a plurality of compaction oligonucleotides and/or hexamine (e.g., cobalt hexamine III) to generate forward extension strands. Individual forward extension strands can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 μM or smaller.

In some embodiments, step (c) of the methods for the pairwise sequencing, the condition suitable to hybridize the plurality of soluble forward sequencing primers to the plurality of retained immobilized single stranded nucleic acid template molecules comprises hybridizing retained immobilized template molecules with the soluble sequencing primers in the presence of a primer extension polymerase, a plurality of nucleotides, and a high efficiency hybridization buffer. In some embodiment, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In some embodiments, step (c) of the methods for the pairwise sequencing comprises: (i) removing the plurality of first forward sequencing read products while retaining the immobilized template molecules; and (ii) contacting the plurality of retained immobilized molecules with a plurality of soluble amplification primers, a plurality of nucleotides (e.g., a second plurality of nucleotides) and a plurality of primer extension polymerases, under a condition suitable to hybridize the plurality of soluble amplification primers to the plurality of retained immobilized template molecules and suitable for conducting polymerase-catalyzed primer extension reactions thereby generating a plurality of forward extension strands, wherein the soluble amplification primers hybridize with the soluble amplification primer binding sequence in the retained immobilized molecules. The primer extension reaction can optionally include a plurality of compaction oligonucleotides and/or hexamine (e.g., cobalt hexamine III) to generate forward extension strands. Individual forward extension strands can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 μM or smaller.

In some embodiments, in step (c) of the methods for the pairwise sequencing, the condition suitable to hybridize the plurality of soluble amplification primers to the plurality of retained immobilized single stranded nucleic acid template molecules comprises hybridizing retained immobilized template molecules with the soluble amplification primers in the presence of a primer extension polymerase, a plurality of nucleotides, and a high efficiency hybridization buffer. In some embodiments, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In some embodiments, in step (c) of the methods for the pairwise sequencing, the plurality of first forward sequencing read products can be removed using an enzyme or a chemical reagent. For example, the plurality of first forward sequencing read products can be enzymatically degraded using a 5′ to 3′ double-stranded DNA exonuclease, including T7 exonuclease (e.g., from New England Biolabs®, catalog #M0263S). In some embodiments, the 5′ to 3′ exonuclease comprises a lambda exonuclease. In some embodiments, the lambda exonuclease can degrade the 5′ phosphorylated end of one strand of double-stranded DNA. In some embodiments, the lambda exonuclease is from New England Biolabs® (e.g., catalog No. M0262S). In some embodiments, the exonuclease degrades double-stranded DNA in a 3′ to 5′ direction, such as for example E. coli exonuclease I (e.g., catalog No. M0568 from New England Biolabs®) or E. coli exonuclease III (e.g., catalog No. M0206S from New England Biolabs®). In some embodiments, the exonuclease lacks endonuclease activity. In some embodiments, the plurality of first forward sequencing read products can be removed with a temperature that favors nucleic acid denaturation.

In some embodiments, in step (c) of the methods for the pairwise sequencing, it is advantageous to employ an exonuclease that degrades double-stranded DNA in a 5′ to 3′ direction when the template molecules to be sequenced comprise single-stranded linear DNA template molecules that are immobilized to a support by their 5′ ends and having their 3′ ends free, and the sequencing read products each comprise a sequencing primer which is annealed to a portion of a given template molecule and subjected to a polymerase-catalyzed primer extension reaction. A 5′ to 3′ exonuclease enzyme, such as T7 exonuclease or lambda exonuclease, can be removed from the template molecule with a washing buffer at a temperature of about 22-25° C. or about 25-30° C. without the need for heat deactivation. By contrast, heat deactivation is required to inactivate certain exonucleases having 3′ to 5′ exonuclease activity. For example, E. coli exonuclease III must be heat deactivated at 70° C., and E. coli exonuclease I must be heat deactivated at 80° C. When DNA molecules are subjected to a pairwise sequencing workflow where a first region is sequenced and the first sequencing read product is removed prior to sequencing a second region, the DNA template molecules can be damaged when subjected to enzyme deactivation conditions using high heat.

In some embodiments, in step (c) of the methods for the pairwise sequencing, a denaturation reagent can be used to remove the plurality of first forward sequencing read products, wherein the denaturation reagent comprises any one or any combination of compounds such as formamide, acetonitrile, guanidinium chloride and/or a pH buffering agent (e.g., Tris-HCl, MES, HEPES, MOPS, or the like). Optionally, the denaturation reagent can further comprise PEG.

In some embodiments, in step (c) of the methods for the pairwise sequencing, the plurality of first forward sequencing read products can be removed using an elevated temperature (e.g., heat) with or without a nucleic acid denaturation reagent. The plurality of first forward sequencing read products can be subjected to a temperature of about 45-50° C., or about 50-60° C., or about 60-70° C., or about 70-80° C., or about 80-90° C., or about 90-95° C., any range therebetween, or higher temperatures.

In some embodiments, in step (c) of the methods for the pairwise sequencing, the plurality of first forward sequencing read products can be removed using formamide (e.g., 40-100% formamide, or any range therebetween) at a temperature of about 65° C. for about 3 minutes, and washing with a reagent comprising about 50 mM NaCl or equivalent ionic strength and having a pH of about 6.5-8.5.

In some embodiments, in the methods for the pairwise sequencing, the primer extension polymerase of step (c) comprises a high-fidelity polymerase. In some embodiments, the primer extension polymerase of step (c) comprises a DNA polymerase capable of catalyzing a primer extension reaction using a uracil-containing template molecule (e.g., a uracil-tolerant polymerase). Exemplary polymerases include, but are not limited to, Q5U Hot Start high-fidelity DNA polymerase (e.g., catalog #M0515S from New England Biolabs®), Taq DNA polymerase, One Taq DNA polymerase (e.g., mixture of Taq and Deep Vent DNA polymerases, catalog #M0480S from New England Biolabs®), LongAmp Taq DNA polymerase (e.g., catalog #M0323S from New England Biolabs®), Epimark Hot Start Taq DNA polymerase (e.g., catalog #M0490S from New England Biolabs®), Bst DNA polymerase (e.g., large fragment, catalog #M0275S from New England Biolabs®), Bsu DNA polymerase (e.g., large fragment, catalog #M0330S from New England Biolabs®), Phi29 DNA polymerase (e.g., catalog #M0269S from New England Biolabs®), E. coli DNA polymerase (e.g., catalog #M0209S from New England Biolabs®), Therminator DNA polymerase (e.g., catalog #M0261S from New England Biolabs®), Vent DNA polymerase and Deep Vent DNA polymerase.

The pairwise methods described herein can provide increased accuracy in a downstream sequencing reaction because step (c) replaces the first forward sequencing read products that were generated in step (b) with forward extension strands having reduced base errors. The first forward sequencing read products are generated in step (b), and may or may not contain erroneously incorporated nucleotides due to polymerase-catalyzed mis-paired bases. When step (c) is conducted with a high-fidelity DNA polymerase, the resulting forward extension strands may have reduced base errors compared to the first forward sequencing read products. The forward extension strands can be used as a nucleic acid template for a downstream sequencing step (e.g., see step (e) below). Thus, step (c) can increase the sequencing accuracy of the downstream step (e) and therefore increase the overall sequencing accuracy of the pairwise sequencing workflow.

In some embodiments, the pairwise sequencing method further comprises step (d): removing the retained immobilized template molecules by generating abasic sites in the immobilized single stranded template molecules at the nucleotide(s) having the scissile moiety and generating gaps at the abasic sites to generate a plurality of gap-containing single stranded nucleic acid template molecules while retaining the plurality of forward extension strands and retaining the plurality of immobilized surface primers (FIG. 9E).

The abasic sites are generated on the retained template strands that contain nucleotides having scissile moieties. In some embodiments, the scissile moieties in the retained template molecules comprises uridine, 8-oxo-7,8-dihydroguanine (e.g., 8oxoG) or deoxyinosine. The abasic sites can be removed to generate a plurality of single stranded nucleic acid template molecules having gaps while retaining the plurality of forward extension strands. The abasic sites can be generated by contacting the immobilized template molecules with an enzyme that removes the nucleo-base at the nucleotide having the scissile moiety. The uracils in the retained template strands can be converted to an abasic site using uracil DNA glycosylase (UDG). The 8oxoG in the retained template strands can be converted to an abasic site using FPG glycosylase. The deoxyinosine in the retained template strands can be converted to an abasic site using AlkA glycosylase.

In some embodiments, in step (d) of the methods for the pairwise sequencing, the gaps can be generated by contacting the abasic sites in the immobilized template molecules with an enzyme or a mixture of enzymes having lyase activity that breaks the phosphodiester backbone at the 5′ and 3′ sides of the abasic site to release the base-free deoxyribose and generate a gap (FIG. 9E). The abasic sites can be removed using AP lyase, Endo IV endonuclease, FPG glycosylase/AP lyase, Endo VIII glycosylase/AP lyase. In some embodiments, generating the abasic sites and removal of the abasic sites to generate gaps can be achieved using a mixture of uracil DNA glycosylase and DNA glycosylase-lyase endonuclease VIII, for example USER® (Uracil-Specific Excision Reagent Enzyme from New England Biolabs®) or thermolabile USER® (also from New England Biolabs®).

In some embodiments, in step (d) of the methods for the pairwise sequencing, the plurality of gap-containing template molecules can be removed using an enzyme, chemical and/or heat. After the gap-removal procedure, the plurality of retained forward extension strands (e.g., see FIG. 9F) are hybridized to the retained immobilized surface primers.

For example, the plurality of gap-containing template molecules can be enzymatically degraded using a 5′ to 3′ double-stranded DNA exonuclease, including T7 exonuclease (e.g., from New England Biolabs®, catalog #M0263S) or lambda exonuclease (e.g., from New England Biolabs®, catalog No. M0262S). When a 5′ to 3′ double-stranded DNA exonuclease is used for removing gap-containing template molecules, then the plurality of soluble amplification primers in step (c) can comprise at least one phosphorothioate diester bond at their 5′ ends which can render the soluble amplification primers resistant to exonuclease degradation. In some embodiments, the plurality of soluble amplification primers in step (c) comprise 2-5 or more consecutive phosphorothioate diester bonds at their 5′ ends. In some embodiments, the plurality soluble amplification primers in step (c) comprise at least one ribonucleotide and/or at least one 2′-O-methyl or 2′-O-methoxyethyl (MOE) nucleotide which can render the forward sequencing primers resistant to exonuclease degradation.

In some embodiments, the plurality of gap-containing template molecules can be removed using a chemical reagent that favors nucleic acid denaturation. The denaturation reagent can include any one or any combination of compounds such as formamide, acetonitrile, guanidinium chloride and/or a buffering agent (e.g., Tris-HCl, MES, HEPES, or the like).

In some embodiments, the plurality of gap-containing template molecules can be removed using an elevated temperature (e.g., heat) with or without a nucleic acid denaturation reagent. The gap-containing template molecules can be subjected to a temperature of about 45-50° C., or about 50-60° C., or about 60-70° C., or about 70-80° C., or about 80-90° C., or about 90-95° C., any range therebetween, or a higher temperature.

In some embodiments, the plurality of gap-containing template molecules can be removed using formamide (e.g., 40-100% formamide) at a temperature of about 65° C. for about 3 minutes, and washing with a reagent comprising about 50 mM NaCl or equivalent ionic strength, and having a pH of about 6.5-8.5.

In some embodiments, the pairwise sequencing method further comprises step (e): sequencing the plurality of retained forward extension strands thereby generating a plurality of first reverse sequencing read products. In some embodiments, the sequencing of step (e) comprises contacting the plurality of retained forward extension strands with a plurality of soluble reverse sequencing primers under a condition suitable to hybridize the reverse sequencing primers to the reverse sequencing primer binding site of the retained forward extension strands, and by conducting sequencing reactions using the hybridized reverse sequencing primers wherein the reverse sequencing reactions generate a plurality of first reverse sequencing read products (FIG. 9G). The first reverse sequencing read products may be hybridized to the retained forward extension strand. Individual retained forward extension strands are hybridized to a first surface primer. The first reverse sequencing read products may not be hybridized to the first surface primer, or covalently joined to the first surface primer. Therefore, the first reverse sequencing read products are not immobilized to the support.

For the sake of simplicity, FIGS. 9F-G show exemplary retained forward extension strands each having two copies of the sequence of interest and various universal primer binding sites. The skilled artisan will appreciate that the retained forward extension strand can include two or more tandem copies containing the sequence of interest and various universal primer binding sites. Therefore, the reverse sequencing reaction can generate a plurality of first reverse sequencing read products that are hybridized to the same retained forward extension strand.

In some embodiments, in step (e) of the methods for the pairwise sequencing, the condition suitable to hybridize the reverse sequencing primers to the reverse sequencing primer binding sequences of the retained forward extension strands comprises contacting the plurality of soluble reverse sequencing primers and the retained forward extension strands with a high efficiency hybridization buffer. In some embodiments, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In an alternative embodiment, the sequencing of step (e) comprises using the immobilized surface primer as a sequencing primer, and conducting sequencing reactions to generate a plurality of first reverse sequencing reads products.

In some embodiments, in the methods for the pairwise sequencing, the reverse sequencing reactions of step (e) comprises contacting the plurality of soluble reverse sequencing primers with the reverse sequencing primer binding sequences of the retained forward extension strands, one or more types of sequencing polymerases, and a plurality of nucleotides or a plurality of multivalent molecules. In some embodiments, the soluble reverse sequencing primers comprise 3′ OH extendible ends. In some embodiments, the soluble reverse sequencing primers comprise a 3′ blocking moiety which can be removed to generate a 3′ OH extendible end. In some embodiments, the soluble reverse sequencing primers lack a nucleotide having a scissile moiety. Exemplary sequencing reactions that employ nucleotides and/or multivalent molecules is described in more detail below. The reverse sequencing reactions can generate a plurality of first reverse sequencing read products. In some embodiments, individual retained forward extension strands have multiple copies of the reverse sequencing primer binding sequences/sites, wherein each reverse sequencing primer binding site is capable of hybridizing to a reverse sequencing primer. Individual reverse sequencing primer binding sites in a given retained forward extension strand can be hybridized to a reverse sequencing primer and can undergo a sequencing reaction. Thus, an individual retained forward extension strand can undergo two or more sequence reactions, where each sequencing reaction is initiated from a reverse sequencing primer that is hybridized to a reverse sequencing primer binding site (e.g., see FIG. 9G). In some embodiments, the sequencing reactions comprise a plurality of nucleotides (or analogs thereof) labeled with a detectable reporter moiety. In some embodiments, the sequencing reaction comprise a plurality of multivalent molecules having nucleotide units, where the multivalent molecules are labeled with a detectable reporter moiety. In some embodiments, the detectable reporter moiety comprises a fluorophore.

In some embodiments, in the methods for the pairwise sequencing, at least one washing step can be conducted after any of steps (a)-(e). The washing step can be conducted with a wash buffer comprising a pH buffering agent, a metal chelating agent, a salt, and a detergent.

In some embodiments, in the methods for the pairwise sequencing, the pH buffering compound in the wash buffer comprises any one or any combination of two or more of Tris, Tris-HCl, Tricine, Bicine, Bis-Tris propane, HEPES, MES, MOPS, MOPSO, BES, TES, CAPS, TAPS, TAPSO, ACES, PIPES, ethanolamine (i.e., 2-amino methanol; MEA), a citrate compound, a citrate mixture, NaOH and/or KOH. In some embodiments, the pH buffering agent can be present in the wash buffer at a concentration of about 1-100 mM, or about 10-50 mM, or about 10-25 mM, or any range therebetween. In some embodiments, the pH of the pH buffering agent which is present in any of the reagents described here in can be adjusted to a pH of about 4-9, or a pH of about 5-9, or a pH of about 5-8, or any range therebetween.

In some embodiments, in the methods for the pairwise sequencing, the metal chelating agent in the wash buffer comprises EDTA (ethylenediaminetetraacetic acid), EGTA (ethylene glycol tetraacetic acid), HEDTA (hydroxyethylethylenediaminetriacetic acid), DPTA (diethylene triamine pentaacetic acid), NTA (N,N-bis(carboxymethyl)glycine), citrate anhydrous, sodium citrate, calcium citrate, ammonium citrate, ammonium bicitrate, citric acid, potassium citrate, or magnesium citrate. In some embodiments, the wash buffer comprises a chelating agent at a concentration of about 0.01-50 mM, or about 0.1-20 mM, or about 0.2-10 mM, or any range therebetween.

In some embodiments, in the methods for the pairwise sequencing, the salt in the wash buffer comprises NaCl, KCl, NH₂SO₄ or potassium glutamate. In some embodiments, the detergent comprises an ionic detergent such as SDS (sodium dodecyl sulfate). The wash buffer can include a monovalent salt at a concentration of about 25-500 mM, or about 50-250 mM, or about 100-200 mM, or any range therebetween.

In some embodiments, in the methods for the pairwise sequencing, the detergent in the wash buffer comprises a non-ionic detergent such as Triton X-100, Tween 20, Tween 80 or Nonidet P-40. In some embodiments, the detergent comprises a zwitterionic detergent such as CHAPS (3-[(3-cholamidopropyl)dimethylammonio]-1-propanesulfonate) or N-Dodecyl-N,N-dimethyl-3-amonio-1-propanesulfate (DetX). In some embodiments, the detergent comprises LDS (lithium dodecyl sulfate), sodium taurodeoxycholate, sodium taurocholate, sodium glycocholate, sodium deoxycholate or sodium cholate. In some embodiments, the detergent is included in the wash buffer at a concentration of about 0.01-0.05%, or about 0.05-0.1%, or about 0.1-0.15%, or about 0.15-0.2%, or about 0.2-0.25%, or any range therebetween.

In some embodiments, in step (b) of the method for pairwise sequencing, the plurality of immobilized template molecules can be sequenced by separately hybridizing soluble sequencing primers (e.g., a first, second, third and fourth forward sequencing primers) to the plurality of immobilized template molecules, conducting separate sequencing reactions to generate forward sequencing read products (e.g., first, second, third, and fourth forward sequencing read products), and removing the forward sequencing read products from the immobilized template molecules by degradation using an enzyme-base reagent in a manner similar to any of the enzyme-based methods described herein, thereby sequencing one or more regions of individual template molecules.

In some embodiments, in step (b) of the method for pairwise sequencing, the plurality of immobilized template molecules comprise: (1) hybridizing at least one first forward sequencing primer to a first region of individual immobilized template molecules in the plurality of template molecules and conducting one or more sequencing reactions (e.g., conducting one or more sequencing cycle reactions) thereby generating a plurality of first extension duplexes each comprising a first sequencing read product hybridized to a template molecule; and (2) contacting the first extension duplexes with a double-stranded DNA specific exonuclease under a condition suitable to degrade the plurality of first sequencing read products and retain the template molecules thereby removing at least a portion of the first sequencing read products from the first region of the template molecules.

In some embodiments, step (b) of the methods for pairwise sequencing further comprise: (3) hybridizing at least one second forward sequencing primer to a second region of individual immobilized template molecules in the plurality of template molecules and conducting one or more sequencing reactions (e.g., conducting one or more sequencing cycle reactions) thereby generating a plurality of second extension duplexes each comprising a second sequencing read product hybridized to a template molecule; and (4) contacting the plurality of second extension duplexes with a double-stranded DNA specific exonuclease under a condition suitable to degrade the plurality of second sequencing read products and retain the template molecules thereby removing at least a portion of the second sequencing read products from the second region of the template molecules.

In some embodiments, the first and second sequencing primers hybridize to different and non-overlapping regions of the same immobilized template molecule. In some embodiments, the first and second sequencing primers hybridize to the same region of the same immobilized template molecule. In some embodiments, the first and second sequencing primers hybridize to overlapping regions of the same immobilized template molecule.

In some embodiments, step (b) of the methods for pairwise sequencing further comprise: (5) hybridizing at least one third forward sequencing primer to a third region of individual immobilized template molecules in the plurality of template molecules and conducting one or more sequencing reactions (e.g., conducting one or more sequencing cycle reactions) thereby generating a plurality of third extension duplexes each comprising a third sequencing read product hybridized to a template molecule; and (6) contacting the plurality of third extension duplexes with a double-stranded DNA specific exonuclease under a condition suitable to degrade the plurality of third sequencing read products and retain the template molecules thereby removing at least a portion of the third sequencing read products from the third region of the template molecules.

In some embodiments, the second and third sequencing primers hybridize to different and non-overlapping regions of the same immobilized template molecule. In some embodiments, the second and third sequencing primers hybridize to the same region of the same immobilized template molecule. In some embodiments, the second and third sequencing primers hybridize to overlapping regions of the same immobilized template molecule.

In some embodiments, step (b) of the method for pairwise sequencing further comprises repeating steps (5) and (6) using a fourth forward sequencing primer that hybridizes to a fourth region of individual template molecules in the plurality of template molecules. The skilled artisan recognizes that steps (5) and (6) can be repeated multiple times using a fourth, fifth, sixth, seventh, eighth, ninth, tenth or more sequencing primers that hybridizes to their respective regions of individual template molecules.

In some embodiments, the third and fourth sequencing primers hybridize to different and non-overlapping regions of the same immobilized template molecule. In some embodiments, the third and fourth sequencing primers hybridize to the same region of the same immobilized template molecule. In some embodiments, the third and fourth sequencing primers hybridize to overlapping regions of the same immobilized template molecule.

In some embodiments, in step (e) of the method for pairwise sequencing, the plurality of retained forward extension strands can be sequenced by separately hybridizing soluble sequencing primers (e.g., a first, second, third and fourth reverse sequencing primers) to the plurality of retained forward extension strands, conducting separate sequencing reactions to generate reverse sequencing read products (e.g., first, second, third, and fourth reverse sequencing read products), and removing the reverse sequencing read products from the retained forward extension strands by degradation using an enzyme-base reagent in a manner similar to the enzyme-based methods described above for steps (1)-(6), thereby sequencing one or more regions of individual retained forward extension strands.

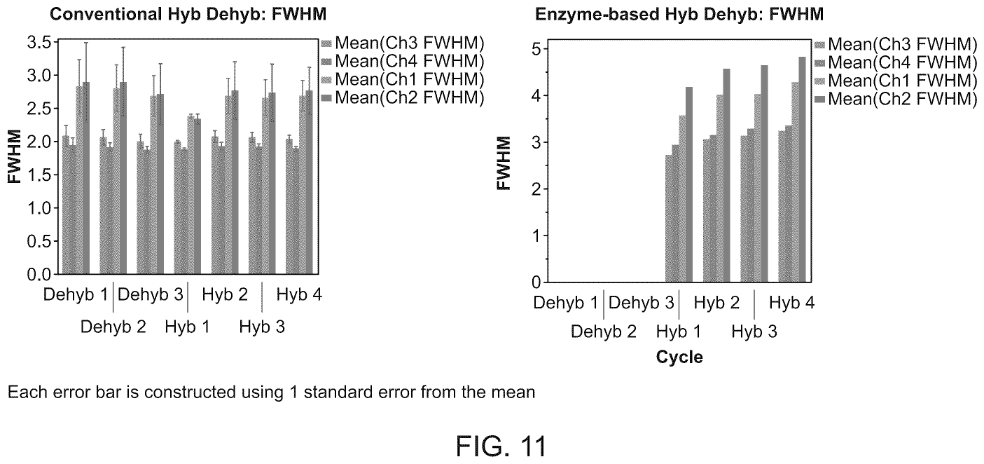

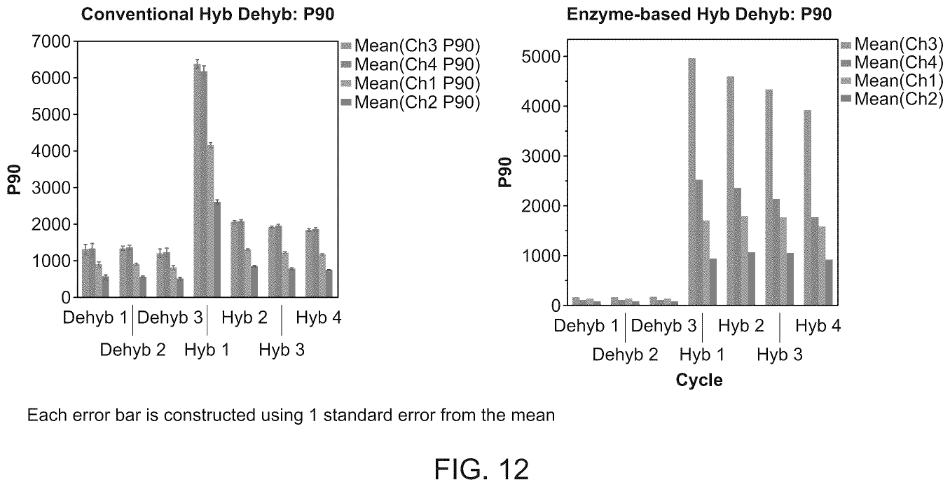

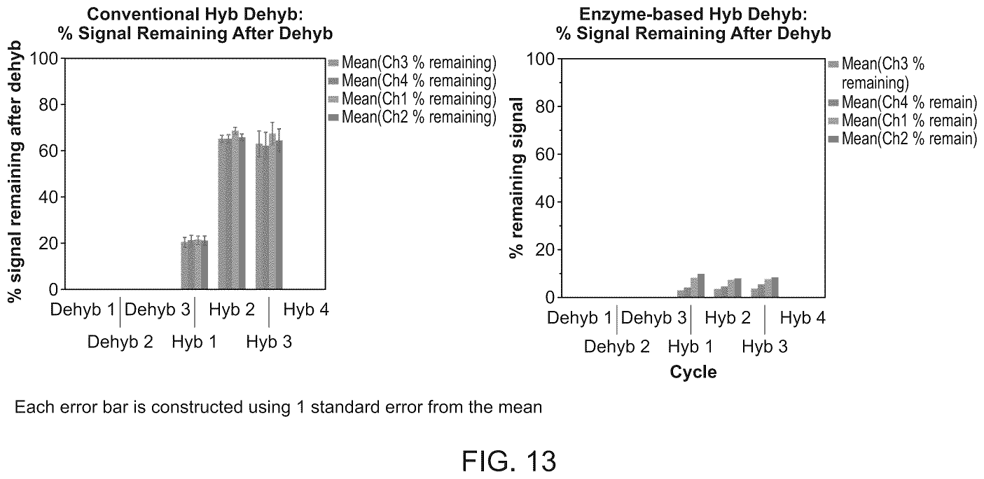

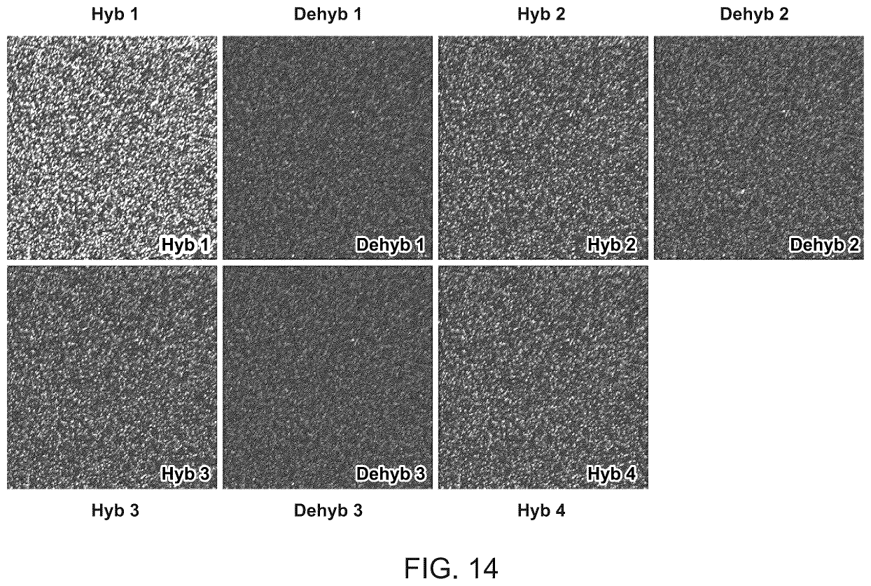

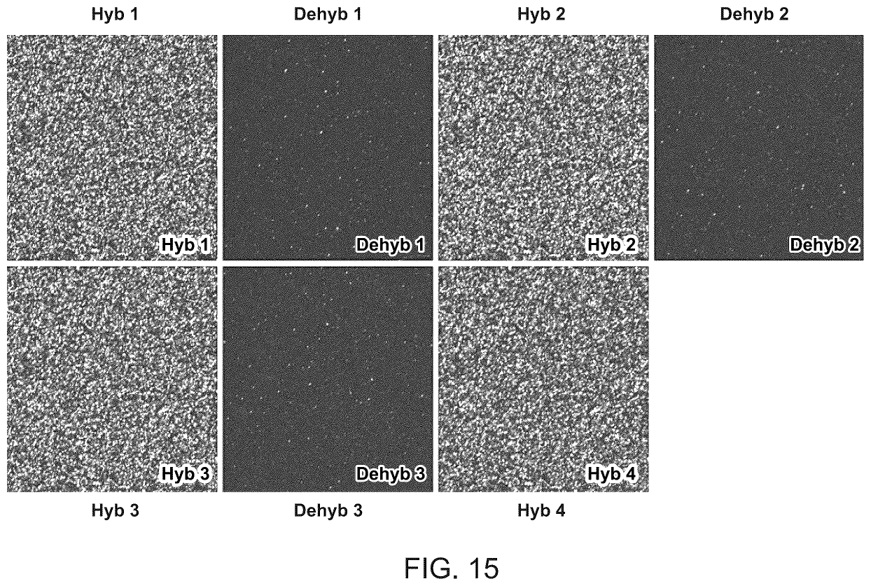

In any of the methods for sequencing described herein, the sequencing methods that employ enzyme-based reagents degrade/remove a higher percentage of the sequencing read products from the template molecules compared to traditional sequencing methods using a chemical-based denaturing reagent (e.g., comprising NaOH and/or formamide) at an elevated temperature (e.g., a temperature of about 50-70° C.) to remove the sequencing read product. See FIGS. 11-15. Thus, the enzyme-based reagents efficiently remove the sequencing read products and reduces residual signals from a previous sequencing cycle while preserving intact template molecules over multiple sequencing cycles.

In some embodiments, in any of the methods for sequencing described herein, the double-stranded DNA specific exonuclease of steps (c), (e) and (g) comprises a double-stranded DNA specific exonuclease having 5′ to 3′ activity. In some embodiments, the enzyme comprises an exonuclease that initiates degradation at the 5′ terminus of double-stranded DNA including linear and circular DNA molecules and nicked double-stranded DNA molecules. The exonuclease degrades double-stranded DNA in a 5′ to 3′ direction and can release mononucleotides and oligonucleotides. The exonuclease comprises a T7 exonuclease encoded by T7 phage gene 6. T7 exonuclease gene 6 is commercially-available from New England Biolabs® (e.g., catalog No. M0263S) and Thermo Fisher Scientific® (e.g., catalog No. 70025Z10KU). In some embodiments, the exonuclease comprises a lambda exonuclease. In some embodiments, the lambda exonuclease can degrade the 5′ phosphorylated end of one strand of double-stranded DNA. In some embodiments, the lambda exonuclease is from New England Biolabs® (e.g., catalog No. M0262S). In some embodiments, the exonuclease degraded double-stranded DNA in a 3′ to 5′ direction, such as for example E. coli exonuclease I or E. coli exonuclease III. In some embodiments, the 5′ to 3′ exonuclease and the 3′ to 5′ exonuclease lack endonuclease activity.

In some embodiments, in any of the methods for sequencing described herein, the first, second, third and fourth extension duplexes of steps (c), (e) and (g), are contacted with the double-stranded DNA specific exonuclease at a concentration of about 5-400 U/mL, or about 10-200 U/mL, or about 20-100 U/mL, or about 40-80 U/mL, or any range therebetween.

In some embodiments, in any of the methods for sequencing described herein, the enzymatic degradation of steps (c), (e) and (g), can be conducted at a temperature of about 20-40° C., or about 25-37° C., or about 28-38° C., or any range therebetween.

In some embodiments, in any of the methods for sequencing described herein, the enzymatic degradation of steps (c), (e) and (g), can be conducted for about 2-20 minutes, or any range therebetween.

In some embodiments, in any of the methods for sequencing described herein, the double-stranded DNA specific exonuclease is formulated in an enzyme-based reagent comprising a double-stranded DNA specific exonuclease, at least one aqueous solvent, a pH buffering agent, a monovalent salt, a divalent salt, and enzyme stabilizer (e.g., BSA). In some embodiments, the aqueous solvent comprises water. In some embodiments, the monovalent salt comprises potassium, for example potassium acetate. In some embodiments, the divalent salt comprises magnesium, for example magnesium acetate. In some embodiments, the enzyme stabilizer comprises a protein comprising bovine serum albumin (BSA), beta-lactoglobulin (e.g., from bovine milk), beta-casein (e.g., from bovine milk) or ovalbumin. In some embodiments, the enzyme-bases reagent has a pH of about 6-9, or about 7-9, or any range therebetween. In some embodiments, the enzyme-based reagent further comprises NaCl at a concentration suitable to reduce 5′ flap endonuclease activity of the T7 exonuclease. The enzyme-based reagent can include NaCl at a concentration of about 20-150 mM, or about 20-40 mM, or about 40-60 mM, or about 60-80 mM, or about 80-100 mM, or about 100-120 mM, or about 120-150 mM, or any range therebetween.

In some embodiments, in any of the methods for sequencing described herein, any of the first, second, third and fourth sequencing primers of steps (b), (d) and (f), comprise an oligonucleotide that is capable of hybridizing with a DNA and/or RNA nucleic acid template molecule to form a duplex molecule. The sequencing primers comprise natural nucleotides and/or nucleotide analogs. The sequencing primers comprise recombinant nucleic acid molecules. The sequencing primers may have any length, but typically range from 4-50 nucleotides. The 3′ end of the sequencing primer comprises an extendible 3′ OH moiety which serves as a nucleotide polymerization initiation site in a polymerase-catalyzed primer extension reaction. Alternatively, the 3′ end of the sequencing primer can lack a 3′ OH moiety, or can include a terminal 3′ blocking group that inhibits nucleotide polymerization in a polymerase-catalyzed reaction. Any one nucleotide, or more than one nucleotide, along the length of the sequencing primer can be labeled with a detectable reporter moiety. The sequencing primer can be in solution (e.g., a soluble primer).

In some embodiments, in any of the methods for sequencing described herein, the primer hybridizing of steps (b), (d) and (f), can be conducted using a hybridization reagent which comprises at least one aqueous solvent, a pH buffering agent, a monovalent cation and a chaotropic agent. In some embodiments, the hybridization reagent optionally comprises any one or any combination of two or more of a detergent, a reducing agent, a chelating agent, an alcohol, a zwitterion, a sugar alcohol and/or a crowding agent. In some embodiments, the aqueous solvent comprises water. In some embodiment, the sodium comprises NaCl. In some embodiments, the chaotropic agent comprise SDS (sodium dodecyl sulfate), urea, thiourea, guanidinium chloride, guanidine hydrochloride, guanidine thiocyanate, guanidine isothionate, potassium thiocyanate, lithium chloride, sodium iodide or sodium perchlorate.

In some embodiments, in any of the methods for sequencing described herein, the primer hybridizing of steps (b), (d) and (f), can be conducted at a temperature of about 20-25° C., or about 25-35° C., or about 35-45° C., or about 45-55° C., or about 55-65° C., or about 65-75° C., any range therebetween, or higher temperatures. In some embodiments, the primer hybridizing can be conducted for about 10-60 seconds, or about 1-3 minutes, or about 3-6 minutes, or about 6-9 minutes, or about 9-12 minutes, or any range therebetween, or longer time frames.

In some embodiments, the primer hybridizing of steps (b), (d) and (f), can be conducted at several different temperatures using a “step down” temperature regime. For example, a first primer hybridizing can be conducted at about 65-45° C. A second primer hybridizing can be conducted at about 25-45° C. A third primer hybridizing can be conducted at about 30-15° C. In some embodiments, the primer hybridizing steps can be conducted for the same length of time or different lengths of time. For example, any of the primer hybridizing steps can be conducted for about 10-60 seconds, or about 1-3 minutes, or about 3-6 minutes, or about 6-9 minutes, or about 9-12 minutes, or any range therebetween, or longer time frames.

In some embodiments, in any of the methods for sequencing described herein, any portion of the at least one single-stranded template molecule is immobilized to the support or immobilized to a coating on the support. For example, the 5′ or 3′ end of the template molecule, or an internal portion of the template molecule, is immobilized to the support.

In some embodiments, in any of the methods for sequencing described herein, the 5′ or 3′ end of the at least one single-stranded nucleic acid template molecule is covalently attached to a surface primer which is immobilized to the support or immobilized to a coating on the support. In some embodiments, the 5′ or 3′ region of the at least one single-stranded nucleic acid template molecule is hybridized to a surface primer which is immobilized to the support or immobilized to a coating on the support.

In some embodiments, in any of the methods for sequencing described herein, the at least one single-stranded nucleic acid template molecule can be generated by a clonal amplification workflow. In some embodiments, the at least one single-stranded nucleic acid template molecule was not generated by a clonal amplification workflow.

In some embodiments, in any of the methods for sequencing described herein, the at least one single-stranded nucleic acid template molecule includes at least one uridine nucleotide or lacks a uridine nucleotide.

In some embodiments, in any of the methods for sequencing described herein, the at least one single-stranded nucleic acid template molecule comprises one copy of the sequence-of-interest. For example, the one-copy template molecule can be generated by bridge amplification. In some embodiments, the at least one single-stranded nucleic acid template molecule comprises two or more tandem copies of the sequence-of-interest. For example, the tandem-copy template molecule can comprise a concatemer which can be generated by rolling circle amplification (RCA).

In some embodiments, in any of the methods for sequencing described herein, the at least one single-stranded nucleic acid template molecule comprises at least one sequence-of-interest (e.g., insert region). In some embodiments, the at least one single-stranded nucleic acid template molecule further comprises at least one universal adaptor sequence including any one or any combination of: a first surface primer binding site (e.g., capture primer binding site); a second surface primer binding site (e.g., surface pinning primer binding site); a first sequencing primer binding site (e.g., forward sequencing primer binding site); a second sequencing primer binding site (e.g., reverse sequencing primer binding site); a first amplification primer binding site (e.g., forward amplification primer binding site); a second amplification primer binding site (e.g., reverse amplification primer binding site); a first sample index sequence; a second sample index sequence; a first unique molecular tag sequence; a second unique molecular tag sequence; a first compaction oligonucleotide binding site; and/or a second compaction oligonucleotide binding site.

In some embodiments, in any of the methods for sequencing described herein, the sequencing read products of steps (b), (d) and (f), comprise a sequencing primer (e.g., a nucleic acid oligonucleotide) which is joined to a polynucleotide having a sequence that is complementary to at least a portion of the single-stranded nucleic acid template molecule. At least a portion of the complementary polynucleotide can be generated by any type of sequencing workflow comprising a polymerase-catalyzed primer extension reaction in which the sequence of the single-strand template molecule is determined. At least a portion of the complementary polynucleotide can be generated by any type of polymerase-catalyzed primer extension reaction in which the sequence of the single-strand template molecule is not determined. In some embodiments, the sequencing primer portion of the sequencing read product has an extendible 3′ terminal end or a non-extendible 3′ terminal end. Various methods for polymerase-catalyzed sequencing are described below.

In some embodiments, in any of the methods for sequencing described herein, the support comprises a planar or non-planar support. The support can be solid or semi-solid. In some embodiments, the support can be porous, semi-porous or non-porous. The support can be made of any material, such as glass, plastic or a polymer material.

In some embodiments, in any of the methods for sequencing described herein, the surface of the support can be coated with one or more compounds to produce a passivated layer on the support. In some embodiments, the passivated layer forms a porous or semi-porous layer. In some embodiments, the surface primer of the single-stranded nucleic template molecule can be attached to the passivated layer to immobilize the primer or template molecule to the support. In some embodiments, the support comprises a low non-specific binding surface that enables improved nucleic acid hybridization, amplification and sequencing performance on the support. In general, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid template molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacet-amidylpentyl)acrylamide-co-acrylamide (PAZAM). In some embodiments, the support comprises a surface coating having at least one hydrophilic polymer coating layer and at least one layer of a plurality of oligonucleotides. The hydrophilic polymer coating layer can comprise polyethylene glycol (PEG). The hydrophilic polymer coating layer can comprise branched PEG having at least 4 branches. In some embodiments, the low non-specific binding coating has a degree of hydrophilicity which can be measured as a water contact angle, where the water contact angle is no more than 45 degrees.

In some embodiments, in any of the methods for sequencing described herein, the support comprises a plurality of single-stranded nucleic acid template molecules immobilized to the support or immobilized to a coating on the support. In some embodiments, about 10²-10¹⁵ template molecule are immobilized to the support at different sites on the support. In some embodiments, the plurality of template molecule is immobilized to pre-determined sites (e.g., locations) on the support. In some embodiments, the plurality of template molecules is immobilized to random sites (e.g., locations) on the support. In some embodiments, the plurality of immobilized template molecules is in fluid communication with each other to permit flowing a solution of reagents (e.g., enzymes including polymerases, multivalent molecules, nucleotides and/or divalent cations, and the like) onto the support so that the plurality of immobilized template molecules on the support can be reacted with the solution of reagents in a massively parallel manner.

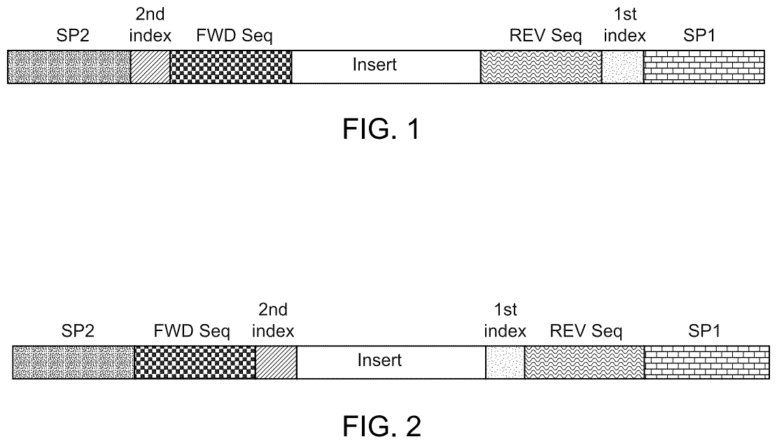

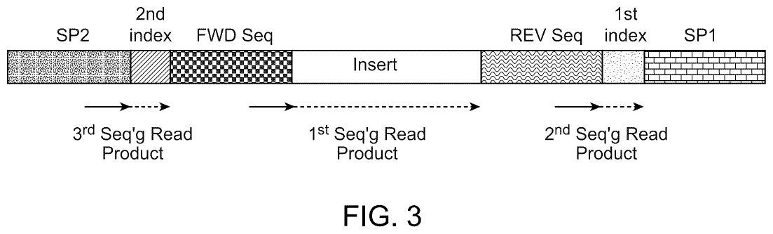

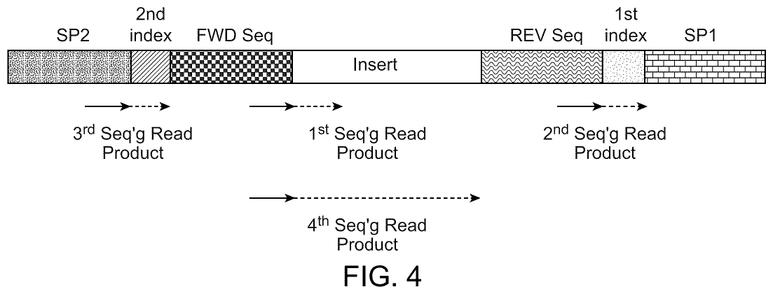

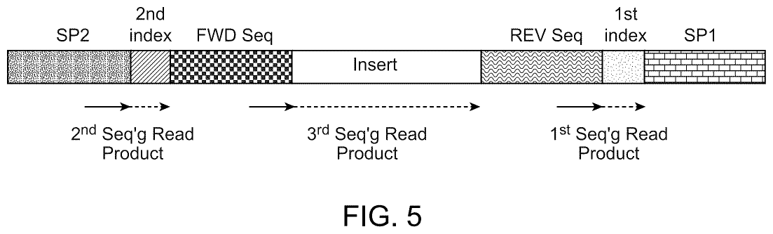

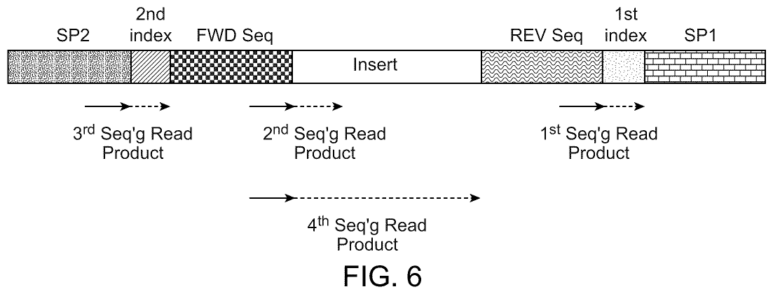

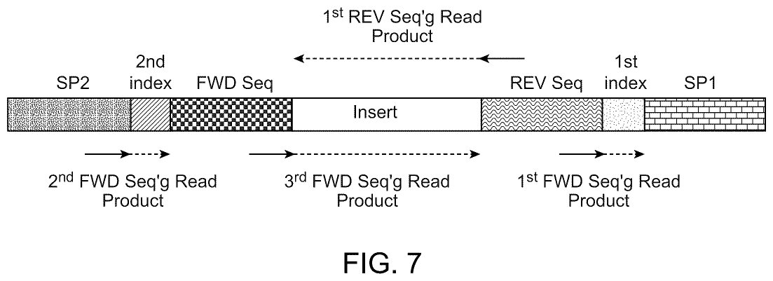

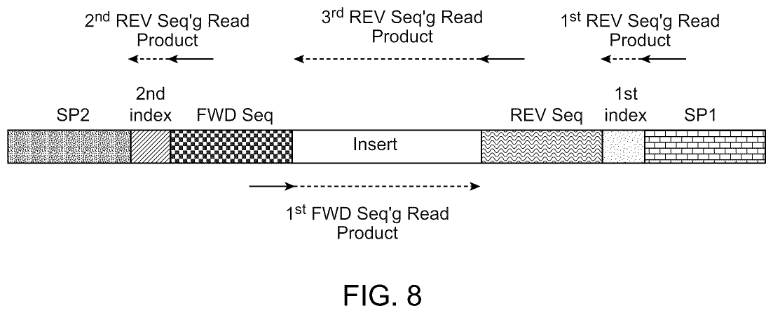

In some embodiments, in any of the methods for sequencing described herein, the at least one single-stranded nucleic acid template molecule comprises sequence-of-interest (e.g., insert region) flanked on both sides by at least one universal adaptor sequence. An exemplary template molecule comprises (ordered from 3′ to 5′; see FIG. 1): first surface primer binding site (e.g., capture primer binding site); a first sample index sequence; a second sequencing primer binding site (e.g., reverse sequencing primer binding site); a sequence-of-interest; a first sequencing primer binding site (e.g., forward sequencing primer binding site); a second sample index sequence; and a second surface primer binding site (e.g., surface pinning primer binding site). The skilled artisan appreciates that many other arrangements of the sequence-of-interest and universal adaptor sequences are possible. The exemplary template molecule can be a repeating unit of a concatemer template molecule having two or more tandem copies of the sequence-of-interest, or the exemplary template molecule can be a single copy template molecule having one copy of the sequence-of-interest.

In some embodiments, a sequencing primer (e.g., first, second, third or fourth sequencing primer) that is employed to sequence the sequence-of-interest region hybridizes to the first sequencing primer binding site (e.g., forward sequencing primer binding site). See for example FIG. 1 or 2.

In some embodiments, a sequencing primer (e.g., first, second, third or fourth sequencing primer) that is employed to sequence the first sample index region hybridizes to a portion of the second sequencing primer binding site (e.g., reverse sequencing primer binding site). See for example FIG. 1 or 2.

In some embodiments, a sequencing primer (e.g., first, second, third or fourth sequencing primer) that is employed to sequence the first sample index region hybridizes to a portion of the reverse sequencing primer binding site. See for example FIG. 1.

In some embodiments, a sequencing primer (e.g., first, second, third or fourth sequencing primer) that is employed to sequence the second sample index region hybridizes to a portion of the second surface primer binding site (e.g., surface pinning primer binding site). See for example FIG. 1.

In some embodiments, the order of sequencing the template molecule comprises: Read 1 (e.g., full length sequencing the sequence-of-interest); and first sample index. In some embodiments, the second sample index is not sequenced. In some embodiments, the second index is sequenced.

In some embodiments, the order of sequencing the template molecule comprises: Read 1 (e.g., full length sequencing the sequence-of-interest); first sample index; and second sample index.

In some embodiments, the order of sequencing the template molecule comprises: Read 1 (e.g., sequencing only a portion of the sequence-of-interest; e.g., 5-25 nucleotides in length, or any range therebetween), first sample index, and second sample index. In some embodiments, sequencing the second sample index is omitted.

In some embodiments, the order of sequencing the template molecule comprises: Read 1 (e.g., sequencing only a portion of the sequence-of-interest; e.g., 5-25 nucleotide in length, or any range therebetween), first sample index, second sample index, and Read 1 (e.g., sequencing full length the sequence-of-interest). In some embodiments, sequencing the second sample index is omitted.

In some embodiments, the order of sequencing the template molecule comprises: first sample index; and Read 1 (e.g., full length sequencing the sequence-of-interest).

In some embodiments, the order of sequencing the template molecule comprises: first sample index; and Read 1 (e.g., sequencing only a portion of the sequence-of-interest; e.g., 5-25 nucleotides in length, or any range therebetween).

In some embodiments, the order of sequencing the template molecule comprises: first sample index; second sample index; Read 1 (e.g., full length sequencing the sequence-of-interest).

In some embodiments, the order of sequencing the template molecule comprises: first sample index; second sample index; Read 1 (e.g., sequencing only a portion of the sequence-of-interest; e.g., 5-25 nucleotides in length, or any range therebetween).

In some embodiments, the order of sequencing the template molecule comprises: first sample index; Read 1 (e.g., full length sequencing the sequence-of-interest); second sample index.

In some embodiments, the order of sequencing the template molecule comprises: first sample index; Read 1 (e.g., sequencing only a portion of the sequence-of-interest; e.g., 5-25 nucleotides in length, or any range therebetween); second sample index; and optionally Read 1 (e.g., full length sequencing the sequence-of-interest).

The skilled artisan will recognize that the sequence-of-interest, first sample index and second sample index can be sequenced in any order. The skilled artisan will recognize that the full length or only a portion of the sequence-of-interest, first sample index and second sample index can be sequenced in any order.

In some embodiments, the template molecule can be sequenced by employing a pairwise sequencing workflow which includes sequencing the first template strand, conducting a primer extension reaction for a second strand synthesis step (e.g., sometimes called a “pairwise turn”), and sequencing a template strand having a sequence that is complementary to the first template strand.

In some embodiments, in a pairwise sequencing workflow, the order of sequencing the template molecule comprises: first sample index; second sample index; Read 1 (e.g., full length sequencing the sequence-of-interest); second strand synthesis (pairwise turn); Read 2 (e.g., full length sequencing the sequence-of-interest).

In some embodiments, in a pairwise sequencing workflow, the order of sequencing the template molecule comprises: Read 1 (e.g., full length sequencing the sequence-of-interest); second strand synthesis (pairwise turn); Read 2 (e.g., full length sequencing the sequence-of-interest); first sample index; second sample index.

In some embodiments, in a pairwise sequencing workflow, the order of sequencing the template molecule comprises: Read 1 (e.g., full length sequencing the sequence-of-interest); second strand synthesis (pairwise turn); first sample index; second sample index; Read 2 (e.g., full length sequencing the sequence-of-interest).

The skilled artisan will recognize that the sequence-of-interest, first sample index and second sample index can be sequenced in any order. The skilled artisan will recognize that the full length or only a portion of the sequence-of-interest, first sample index and second sample index can be sequenced in any order.

### Methods for Sequencing

In some aspects, the present disclosure provides methods for sequencing any of the immobilized concatemer molecules described herein. Any of the methods for conducting rolling circle amplification reaction described herein can be used to generate a plurality of concatemer molecules immobilized to a support, and the immobilized concatemers can be subjected to sequencing reactions. In some embodiments, the sequencing reactions employ detectably labeled nucleotide analogs. In some embodiments, the sequencing reactions employ a two-stage sequencing reaction comprising binding detectably labeled multivalent molecules, and incorporating nucleotide analogs. In some embodiments, the sequencing reactions employ non-labeled nucleotide analogs. The terms concatemer molecule and template molecule are used interchangeably.

In some embodiments, any of the rolling circle amplification reaction described herein (e.g., RCA conducted on-support or in-solution) can be used to generate immobilized concatemers each containing tandem repeat units of the sequence-of-interest and any adaptor sequences present in the covalently closed circular library molecules. For example, the tandem repeat unit comprises: (i) a first left universal adaptor sequence having a binding sequence for a first surface primer, (ii) a second left universal adaptor sequence having a binding sequence for a first sequencing primer, (iii) a sequence-of-interest, (iv) a second right universal adaptor sequence having a binding sequence for a second sequencing primer, (v) a first right universal adaptor sequence having a binding sequence for a second surface primer, and (vii) a first left index sequence and/or a first right index sequence (e.g., see FIGS. 9A and 10). In some embodiments, the tandem repeat unit further comprises a first left unique identification sequence and/or a first right unique identification sequence.

The immobilized concatemer can self-collapse into a compact nucleic acid nanoball. Inclusion of one or more compaction oligonucleotides during the RCA reaction can further compact the size and/or shape of the nanoball. An increase in the number of tandem repeat units in a given concatemer increases the number of sites along the concatemer for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer yields an increased signal intensity for each concatemer. Multiple portions of a given concatemer can be simultaneously sequenced. Furthermore, a plurality of binding complexes can form along a particular concatemer molecule, each binding complex comprising a sequencing polymerase bound to a multivalent molecule wherein the plurality of binding complexes remains stable without dissociation resulting in increased persistence time which increases signal intensity and reduces imaging time.

### Methods for Sequencing Using Nucleotide Analogs

In some aspects, the present disclosure provides methods for sequencing any of the immobilized concatemer molecules described herein, the methods comprising step (a): contacting a sequencing polymerase to (i) a nucleic acid concatemer molecule and (ii) a nucleic acid sequencing primer, wherein the contacting is conducted under a condition suitable to bind the sequencing polymerase to the nucleic acid concatemer molecule which is hybridized to the nucleic acid primer, wherein the nucleic acid concatemer molecule hybridized to the nucleic acid primer forms the nucleic acid duplex. In some embodiments, the sequencing polymerase comprises a recombinant mutant sequencing polymerase that can bind and incorporate nucleotide analogs. In some embodiments, the sequencing primer comprises a 3′ extendible end.

In some embodiments, in the methods for sequencing concatemer molecules, the sequencing primer comprises a 3′ extendible end or a 3′ non-extendible end. In some embodiments, the plurality of nucleic acid concatemer molecules comprise amplified template molecules (e.g., clonally amplified template molecules). In some embodiments, the plurality of nucleic acid concatemer molecules comprise one copy of a target sequence of interest. In some embodiments, the plurality of nucleic acid molecules comprises two or more tandem copies of a target sequence of interest (e.g., as concatemers). In some embodiments, the nucleic acid concatemer molecules in the plurality of nucleic acid concatemer molecules comprise the same target sequence of interest or different target sequences of interest. In some embodiments, the plurality of nucleic acid concatemer molecules and/or the plurality of nucleic acid primers are in solution or are immobilized to a support. In some embodiments, when the plurality of nucleic acid concatemer molecules and/or the plurality of nucleic acid primers are immobilized to a support, the binding with the first sequencing polymerase generates a plurality of immobilized first complexed polymerases. In some embodiments, the plurality of nucleic acid concatemer molecules and/or nucleic acid primers are immobilized to 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) different sites on a support. In some embodiments, the binding of the plurality of concatemer molecules and nucleic acid primers with the plurality of first sequencing polymerases generates a plurality of first complexed polymerases immobilized to 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) different sites on the support.

In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least on nucleotide is labeled with a detectable reporter moiety (e.g., a fluorophore).

In some embodiments, the methods for sequencing further comprise step (c): incorporating at least one nucleotide into the 3′ end of the extendible primer under a condition suitable for incorporating the at least one nucleotide. In some embodiments, the suitable conditions for nucleotide binding the polymerase and for incorporation the nucleotide can be the same or different. In some embodiments, conditions suitable for incorporating the nucleotide comprise inclusion of at least one catalytic cation comprising magnesium and/or manganese. In some embodiments, the at least one nucleotide binds the sequencing polymerase and incorporates into the 3′ end of the extendible primer. In some embodiments, the incorporating the nucleotide into the 3′ end of the primer in step (c) comprises a primer extension reaction.

In some embodiments, the methods for sequencing further comprise step (d): repeating the incorporating at least one nucleotide into the 3′ end of the extendible primer of steps (b) and (c) at least once. In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., a fluorophore) that is attached to the nucleotide can correspond to the nucleotide base (e.g., dATP, dGTP, dCTP, dTTP or dUTP) to permit detection and identification of the nucleotide base. In some embodiments, the method further comprises detecting the at least one incorporated nucleotide at step (c) and/or (d). In some embodiments, the method further comprises identifying the at least one incorporated nucleotide at step (c) and/or (d). In some embodiments, the sequence of the nucleic acid concatemer molecule can be determined by detecting and identifying the nucleotide that binds the sequencing polymerase, thereby determining the sequence of the concatemer molecule. In some embodiments, the sequence of the nucleic acid concatemer molecule can be determined by detecting and identifying the nucleotide that incorporates into the 3′ end of the primer, thereby determining the sequence of the concatemer molecule.

In some embodiments, in the methods for sequencing, the plurality of sequencing polymerases that are bound to the nucleic acid duplexes comprise a plurality of complexed polymerases, having at least a first and second complexed polymerase, wherein (a) the first complexed polymerases comprises a first sequencing polymerase bound to a first nucleic acid duplex comprising a first nucleic acid template sequence which is hybridized to a first nucleic acid primer, (b) the second complexed polymerases comprises a second sequencing polymerase bound to a second nucleic acid duplex comprising a second nucleic acid template sequence which is hybridized to a second nucleic acid primer, (c) the first and second nucleic acid template sequences comprise the same or different sequences, (d) the first and second nucleic acid concatemers are clonally-amplified, (e) the first and second primers comprise extendible 3′ ends or non-extendible 3′ ends, and (f) the plurality of complexed polymerases are immobilized to a support. In some embodiments, the density of the plurality of complexed polymerases is about 10²-10¹⁵ (e.g., 10², 10³, 10⁴, 10¹, 10⁶, 10⁷, 10⁸, 10⁹, 10¹⁰, 10¹¹, 10¹², 10¹³, 10¹⁴, or 10¹⁵) complexed polymerases per mm² that are immobilized to the support.

### Two-Stage Methods for Nucleic Acid Sequencing

In some aspects, the present disclosure provides a two-stage method for sequencing any of the immobilized concatemer molecules described herein. In some embodiments, the first stage generally comprises binding multivalent molecules to complexed polymerases to form multivalent-complexed polymerases, and detecting the multivalent-complexed polymerases.

In some embodiments, the first stage comprises step (a): contacting a plurality of a first sequencing polymerase to (i) a plurality of nucleic acid concatemer molecules and (ii) a plurality of nucleic acid sequencing primers, wherein the contacting is conducted under a condition suitable to bind the plurality of first sequencing polymerases to the plurality of nucleic acid concatemer molecules and the plurality of nucleic acid primers thereby forming a plurality of first complexed polymerases each comprising a first sequencing polymerase bound to a nucleic acid duplex wherein the nucleic acid duplex comprises a nucleic acid concatemer molecule hybridized to a nucleic acid primer. In some embodiments, the first polymerase comprises a recombinant mutant sequencing polymerase. In some embodiments, the sequencing primer comprises a 3′ extendible end.

In some embodiments, in the methods for sequencing concatemer molecules, the sequencing primer comprises a 3′ extendible end or a 3′ non-extendible end. In some embodiments, the plurality of nucleic acid concatemer molecules comprise amplified template molecules (e.g., clonally amplified template molecules). In some embodiments, the plurality of nucleic acid concatemer molecules comprise one copy of a target sequence of interest. In some embodiments, the plurality of nucleic acid molecules comprises two or more tandem copies of a target sequence of interest (e.g., as concatemers). In some embodiments, the nucleic acid concatemer molecules in the plurality of nucleic acid concatemer molecules comprise the same target sequence of interest or different target sequences of interest. In some embodiments, the plurality of nucleic acid concatemer molecules and/or the plurality of nucleic acid primers are in solution or are immobilized to a support. In some embodiments, when the plurality of nucleic acid concatemer molecules and/or the plurality of nucleic acid primers are immobilized to a support, the binding with the first sequencing polymerase generates a plurality of immobilized first complexed polymerases. In some embodiments, the plurality of nucleic acid concatemer molecules and/or nucleic acid primers are immobilized to 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹) different sites on a support. In some embodiments, the binding of the plurality of concatemer molecules and nucleic acid primers with the plurality of first sequencing polymerases generates a plurality of first complexed polymerases immobilized to 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms and each nucleotide arm is attached to a nucleotide (e.g., a nucleotide unit) (e.g., FIGS. 17-21). In some embodiments, the contacting of step (b) is conducted under a condition suitable for binding complementary nucleotide units of the multivalent molecules to at least two of the plurality of first complexed polymerases thereby forming a plurality of multivalent-complexed polymerases. In some embodiments, the condition is suitable for inhibiting polymerase-catalyzed incorporation of the complementary nucleotide units into the primers of the plurality of multivalent-complexed polymerases. In some embodiments, the plurality of multivalent molecules comprises at least one multivalent molecule having multiple nucleotide arms (e.g., FIGS. 17-21) each attached with a nucleotide analog (e.g., a nucleotide analog unit), where the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety. In some embodiments, the detectable reporter moiety comprises a fluorophore. In some embodiments, the contacting of step (b) is conducted in the presence of at least one non-catalytic cation comprising strontium, barium and/or calcium.

In some embodiments, the methods for sequencing further comprise step (c): detecting the plurality of multivalent-complexed polymerases. In some embodiments, the detecting includes detecting the multivalent molecules that are bound to the complexed polymerases, where the complementary nucleotide units of the multivalent molecules are bound to the primers, but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker and/or nucleotide unit of the multivalent molecules.

In some embodiments, the methods for sequencing further comprise step (d): identifying the nucleo-base of the complementary nucleotide units that are bound to the plurality of first complexed polymerases, thereby determining the sequence of the concatemer molecule. In some embodiments, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some embodiments, the second stage of the two-stage sequencing method generally comprises nucleotide incorporation. In some embodiments, the methods for sequencing further comprise step (e): dissociating the plurality of multivalent-complexed polymerases and removing the plurality of first sequencing polymerases and their bound multivalent molecules, and retaining the plurality of nucleic acid duplexes.

In some embodiments, the methods for sequencing further comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) has an amino acid sequence that is 100% identical to the amino acid sequence as the plurality of the second sequencing polymerases of step (f). In some embodiments, the plurality of first sequencing polymerases of step (a) has an amino acid sequence that differs from the amino acid sequence of the plurality of the second sequencing polymerases of step (f).

In some embodiments, the methods for sequencing further comprise step (g): contacting the plurality of second complexed polymerases with a plurality of nucleotides, wherein the contacting is conducted under a condition suitable for binding complementary nucleotides from the plurality of nucleotides to at least two of the second complexed polymerases thereby forming a plurality of nucleotide-complexed polymerases. In some embodiments, the contacting of step (g) is conducted under a condition that is suitable for promoting polymerase-catalyzed incorporation of the bound complementary nucleotides into the primers of the nucleotide-complexed polymerases thereby forming a plurality of nucleotide-complexed polymerases. In some embodiments, the incorporating the nucleotide into the 3′ end of the primer in step (g) comprises a primer extension reaction. In some embodiments, the contacting of step (g) is conducted in the presence of at least one catalytic cation comprising magnesium and/or manganese. In some embodiments, the plurality of nucleotides comprises native nucleotides (e.g., non-analog nucleotides) or nucleotide analogs. In some embodiments, the plurality of nucleotides comprises a 2′ and/or 3′ chain terminating moiety which is removable or is not removable. In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base or is not removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, the methods for sequencing further comprise step (h): detecting the complementary nucleotides which are incorporated into the primers of the nucleotide-complexed polymerases. In some embodiments, the plurality of nucleotides is labeled with a detectable reporter moiety to permit detection. In some embodiments, in the methods for sequencing concatemer molecules, the detecting of step (h) is omitted.

In some embodiments, the methods for sequencing further comprise step (i): identifying the bases of the complementary nucleotides which are incorporated into the primers of the nucleotide-complexed polymerases. In some embodiments, the identification of the incorporated complementary nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid concatemer molecules. In some embodiments, in the methods for sequencing concatemer molecules, the identifying of step (i) is omitted.

In some embodiments, the methods for sequencing further comprise step j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some embodiments, the methods for sequencing further comprise step (k): repeating steps (a)-(j) at least once. In some embodiments, the sequence of the nucleic acid concatemer molecules can be determined by detecting and identifying the multivalent molecules that bind the sequencing polymerases but do not incorporate into the 3′ end of the primer at steps (c) and (d). In some embodiments, the sequence of the nucleic acid concatemer molecule can be determined (or confirmed) by detecting and identifying the nucleotide that incorporates into the 3′ end of the primer at steps (h) and (i).

In some embodiments, in any of the methods for sequencing nucleic acid molecules, the binding of the plurality of first complexed polymerases with the plurality of multivalent molecules forms at least one avidity complex, the method comprising the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. The concatemer template molecule comprises tandem repeat sequences of a sequence of interest and at least one universal sequencing primer binding site. The first and second nucleic acid primers can bind to a sequencing primer binding site along the concatemer template molecule. Exemplary multivalent molecules are shown in FIGS. 17-20.

In some embodiments, in any of the methods for sequencing nucleic acid molecules, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or mutant sequencing polymerase described herein. The concatemer template molecule comprises tandem repeat sequences of a sequence of interest and at least one universal sequencing primer binding site. The plurality of nucleic acid primers can bind to a sequencing primer binding site along the concatemer template molecule. Exemplary multivalent molecules are shown in FIGS. 17-20.

### Sequencing-by-Binding

The present disclosure provides methods for sequencing any of the immobilized concatemer molecules described herein, wherein the sequencing methods comprise a sequencing-by-binding (SBB) procedure which employs non-labeled chain-terminating nucleotides. In some embodiments, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer.

Exemplary sequencing-by-binding methods are described, for example and without limitation, in U.S. Pat. Nos. 10,246,744 and 10,731,141 (where the contents of both patents are hereby incorporated by reference in their entireties).

### Sequencing Polymerases

The present disclosure provides methods for sequencing nucleic acid molecules, where any of the sequencing methods described herein employ at least one type of sequencing polymerase and a plurality of nucleotides, or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a concatemer template molecule. In some embodiments, the sequencing polymerase(s) is/are capable of binding a complementary nucleotide unit of a multivalent molecule opposite a nucleotide in a concatemer template molecule. In some embodiments, the plurality of sequencing polymerases comprises recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N®, VENT®, DEEP VENT®, THERMINATOR®, Pfu, KOD, Pfx, Tgo and RB69 polymerases.

### Nucleotides

In some aspects, the present disclosure provides methods for sequencing nucleic acid molecules, where any of the sequencing methods described herein employ at least one nucleotide. The nucleotides comprise a base, sugar and at least one phosphate group. In some embodiments, at least one nucleotide in the plurality comprises an aromatic base, a five-carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing nucleic acid molecules described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing nucleic acid molecules described herein, at least one nucleotide in the plurality of nucleotides comprises a terminator nucleotide analog having a chain terminating moiety (e.g., a blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, and/or disulfide are cleavable with phosphine, or with a thiol group including but not limited to beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, at least one nucleotide in the plurality of nucleotides comprises a terminator nucleotide analog having a chain terminating moiety (e.g., a blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, in any of the methods for sequencing nucleic acid molecules described herein, the plurality of nucleotides comprises a plurality of nucleotides labeled with a detectable reporter moiety. The detectable reporter moiety can comprise a fluorophore. In some embodiments, the fluorophore is attached to the nucleotide base. In some embodiments, the fluorophore is attached to the nucleotide base with a linker which is cleavable or otherwise removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, and/or disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, the cleavable linker on the nucleotide base comprises cleavable moiety including an azide, azido or azidomethyl group. In some embodiments, the cleavable moieties azide, azido and azidomethyl group are cleavable or otherwise removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

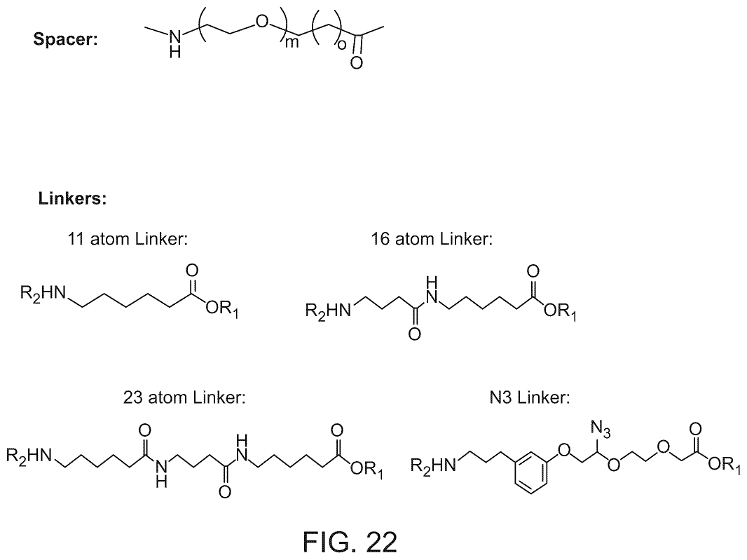

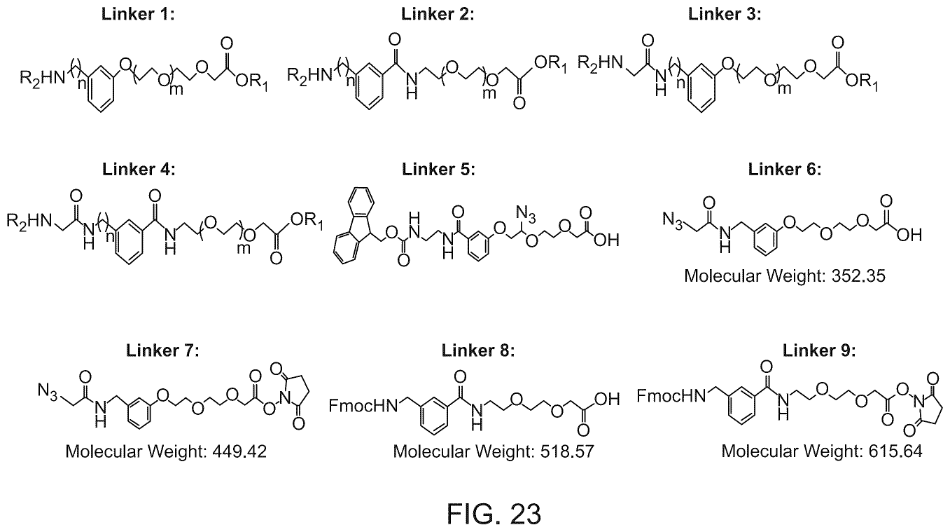

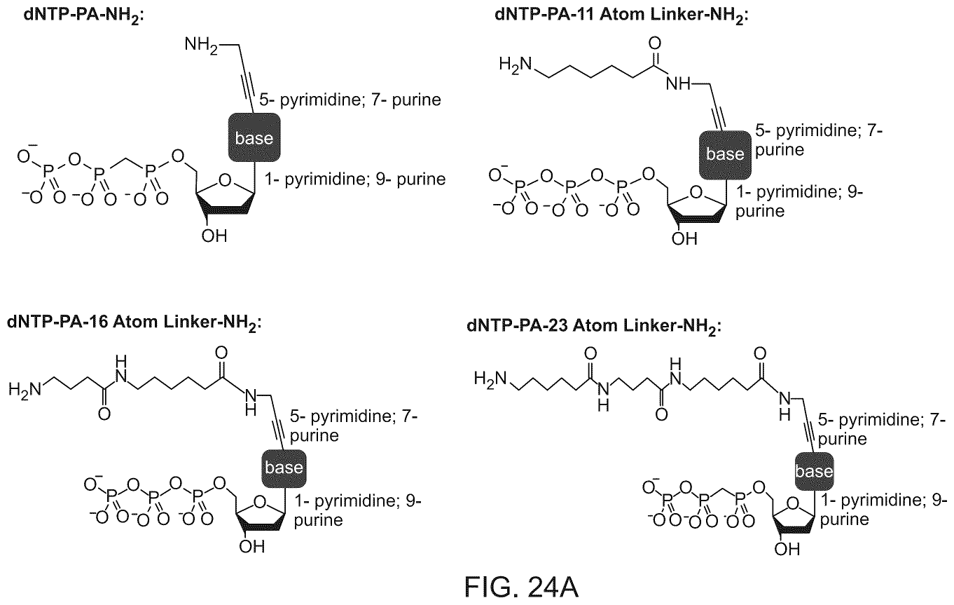

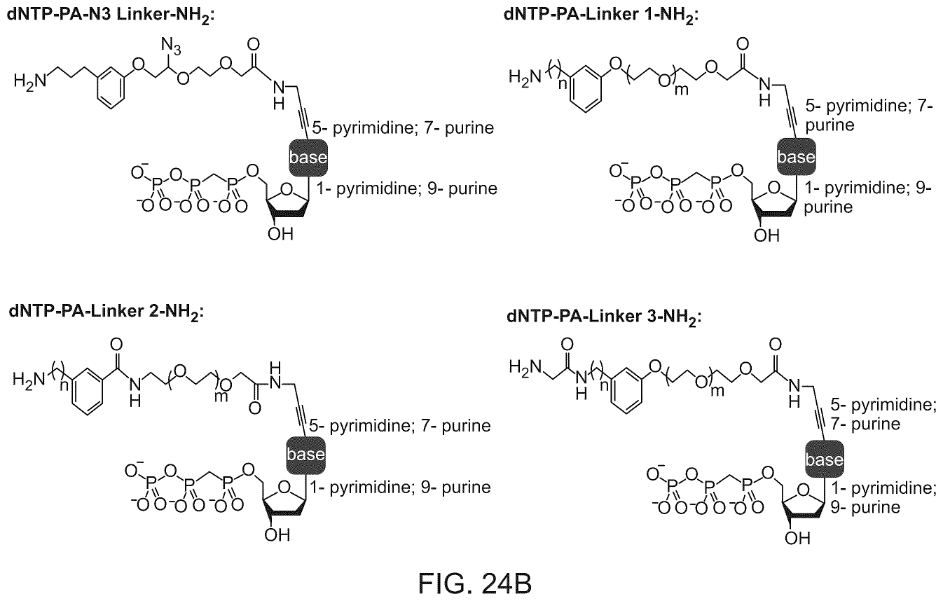

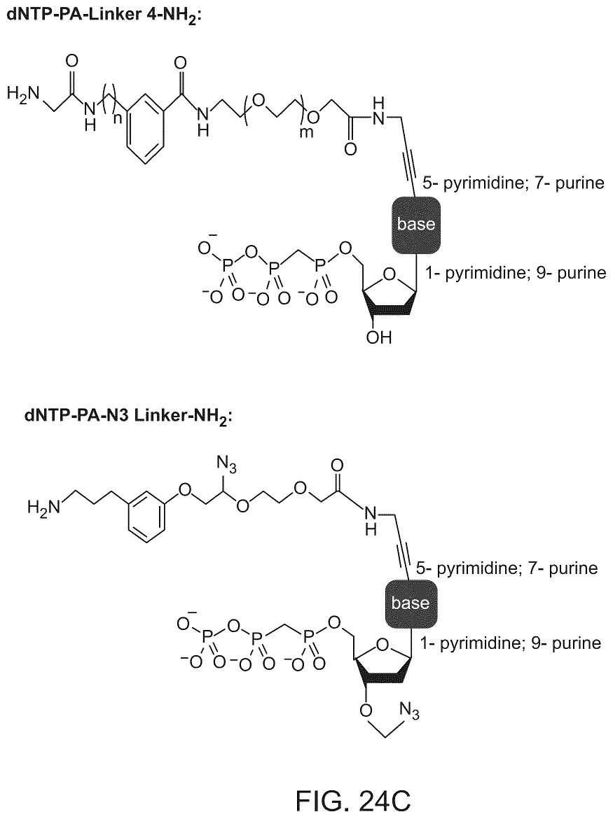

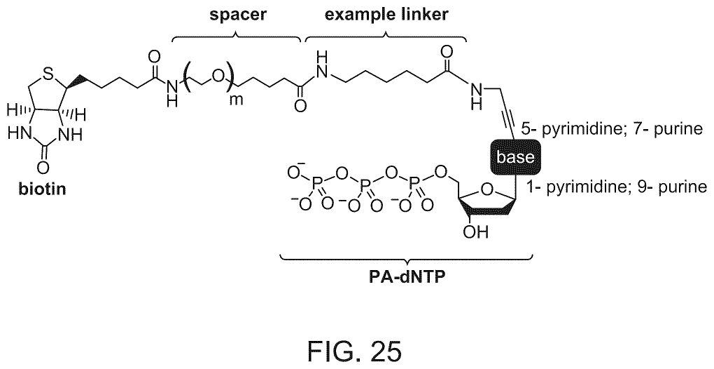

In some aspects, the present disclosure provides methods for sequencing nucleic acid molecules, where any of the sequencing methods described herein employ at least one multivalent molecule. In some embodiments, the multivalent molecule comprises a plurality of nucleotide arms attached to a core and having any configuration including a starburst, helter skelter, or bottle brush configuration (e.g., FIG. 17). In some embodiments, the multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, and wherein the linker is attached to the nucleotide unit. In some embodiments, the nucleotide unit comprises a base, a sugar and at least one phosphate group, and the linker is attached to the nucleotide unit through the base. In some embodiments, the linker comprises an aliphatic chain or an oligo ethylene glycol chain where both linker chains have 2-6 subunits. In some embodiments, the linker also includes an aromatic moiety. An exemplary nucleotide arm is shown in FIG. 21. Exemplary multivalent molecules are shown in FIGS. 17-20. An exemplary spacer is shown in FIG. 22 (top) and exemplary linkers are shown in FIG. 22 (bottom) and FIG. 23. Exemplary nucleotides attached to a linker are shown in FIGS. 24A-C. An exemplary biotinylated nucleotide arm is shown in FIG. 25.

In some embodiments, a multivalent molecule comprises a core attached to multiple nucleotide arms, and wherein the multiple nucleotide arms have the same type of nucleotide unit, which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit can comprise an aromatic base, a five-carbon sugar (e.g., ribose or deoxyribose), and one or more phosphate groups (e.g., 1-10 phosphate groups). The plurality of multivalent molecules can comprise one type of multivalent molecule, having one type of nucleotide unit selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, and/or disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., a fluorophore) that is attached to the multivalent molecule can correspond to the base (e.g. dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises a streptavidin-type or avidin-type moiety which includes an avidin protein, as well as any derivatives, analogs and other non-native forms of avidin that can bind to at least one biotin moiety. Other forms of avidin moieties can include native and recombinant avidin and streptavidin as well as derivatized molecules, e.g., non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN®, CAPTAVIDIN®, NEUTRAVIDIN® and NEUTRALITE AVIDIN®.

In some embodiments, any of the methods for sequencing nucleic acid molecules described herein can include forming a binding complex, where the binding complex comprises (i) a polymerase, a nucleic acid concatemer molecule duplexed with a primer, and a nucleotide, or the binding complex comprises (ii) a polymerase, a nucleic acid concatemer molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Sample Indexes for Improved Base Calling

Generally, it is desirable to prepare nucleic acid libraries to be distributed onto a support (e.g., a coated flowcell), where the library molecules are converted into template molecules that are immobilized at a high density to the support for massively parallel sequencing. For template molecules that are immobilized at high densities at random locations on the support, the challenge of resolving high density fluorescent images for accurate base calling during sequencing runs becomes challenging.

In the present disclosure, nucleotide diversity of a population of immobilized template molecules refers to the relative proportion of nucleotides A, G, C and T that are present in each sequencing cycle. An optimal high diversity library will generally include sequence-of-interest (insert) regions having approximately equal proportions of all four nucleotides represented in each cycle of a sequencing run. A low diversity library will generally include sequence-of-interest (insert) regions having a high proportion of certain nucleotides and low proportion of other nucleotides. To overcome the problem of low diversity libraries, a small amount of a high diversity library prepared from PhiX bacteriophage is typically mixed with the library-of-interest (e.g., PhiX spike-in library) and sequenced together on the same flowcell. While the PhiX library spike-in library provides nucleotide diversity it also occupies space on the flowcell thereby replacing the target libraries carrying the sequence-of-interest and reduces the amount of sequencing data obtainable from the target libraries (e.g., reduces sequencing throughput). Another method to overcome the problem of low diversity libraries is to prepare target library molecules having at least one sample index sequence that is designed to be color-balanced. However, it may be desirable to design a large number of sample index sets, for example and without limitation, a set of single index sample sequences or paired index sample sequences for 16-plex, 24-plex, 96-plex or larger plexy levels. It is challenging to design sample index sequences, as a single or paired sample indexes, for large sample index sets where all of the sample index sequences are color-balanced.

An alternative method to overcome the challenges of sequencing low diversity library molecules (e.g., at high density on the support) is to prepare libraries having at least one sample index sequence comprising a short random sequence (e.g., NNN) linked directly to a universal sample index sequence, where the short random sequence provides nucleotide diversity and color balance. For example, and without limitation, the right and/or left index sequences can include a short random sequence (e.g., NNN) and a universal sample index sequence. The short random sequence (e.g., NNN) can be located 5′ of the universal sample index sequence. In a population of sample-indexed library molecules, the short random sequence of the sample index can provide high nucleotide diversity which includes approximately equal proportions of all four nucleotides (e.g., A, G, C, T and/or U) that will be represented in each cycle of a sequencing run. The high nucleotide diversity of the short random sequence also can provide color balance during each cycle of the sequencing run. Without wishing to be bound by theory, it is postulated that one advantage of designing sample indexes to include a short random sequence (e.g., NNN) is that, in a low-plexy population of library molecules (e.g., 2-plex or 4-plex), the universal sample index sequences that identify the two or four different samples need not exhibit nucleotide diversity. Additionally, the nucleotide diversity of the short random sequence (e.g., NNN) can obviate the need to include a PhiX spike-in library, or permits use of a reduced amount of PhiX spike-in library to be distributed onto the flowcell and sequenced.

The target library molecule can include a single sample index sequence which includes a short random sequence and a universal sample index sequence. In some embodiments, the sequencing data from only the single sample index sequence can be used for polony mapping and template registration because the short random sequence (e.g., NNN) provides sufficient nucleotide diversity and color balance. The sequencing data from the universal sample index sequence can be used to distinguish sequences of interest obtained from different sample sources in a multiplex assay.

The target library molecule can further include a second sample index sequence (e.g., dual sample index) comprising a second universal sample index sequence. In some embodiments, the sequencing data from the first and second sample index sequences (e.g., left and right sample indexes) can be used for polony mapping and/or template registration because the short random sequences provides sufficient nucleotide diversity and color balance. The sequencing data from the first universal sample index sequence and the second universal sample index sequence can be used as dual sample indexes to distinguish sequences of interest obtained from different sample sources in a multiplex assay. In some embodiments, the second sample index sequence may or may not include a second short random sequence (e.g., NNN).

The order of sequencing the sequence-of-interest region and the sample index region(s) can also be used to improve the challenges of sequencing low diversity library molecules. For example, the sample index region can be sequenced first before sequencing the sequence-of-interest region, and the sample index sequence can be associated with the sequence-of-interest region. For example, sample index region can be sequenced first including sequencing the short random sequence (e.g., NNN) and optionally sequencing at least a portion of the universal sample index), and then sequencing the sequence-of-interest region. In a population of sample indexed library molecules, the short random sequence (e.g. NNN) can provide nucleotide diversity which may not be provided the sequence-of-interest regions of the library molecules. The sequence of the sample index can provide improved nucleotide diversity and color balance for polony mapping and template registration.

Additionally, in some embodiments, when sequencing the sample index region first, the length of the sequenced sample index region is relatively short (e.g., less than 30 nucleotides in length). The length of the sequenced sample index region can be short (e.g. less than 30 nucleotides in length) so that de-hybridization of the product of the sequenced sample index region is more complete. Gentler de-hybridization conditions can be used to remove most or all of the product of the sequenced sample index region which reduces the level of residual signals from any sequencing products remaining hybridized to the template molecules. By contrast, in some embodiments, the sequence-of-interest region is much longer than the sample index region (e.g., more than 100 nucleotides in length). When the sequence-of-interest region is sequenced before the sample index region, the product of the sequenced sequence-of-interest region must be subjected to harsher de-hybridization conditions to remove any products remaining hybridized to the template molecules which may damage the template molecules.

The present disclosure provides nucleic acid library molecules (100) each comprising at least one sample index sequence that can be used to distinguish sequences of interest obtained from different sample sources in a multiplex assay, where the at least one sample index sequence comprises a short random sequence (e.g., NNN) linked to a universal sample index sequence. In some embodiments, the left sample index comprises a short random sequence (e.g., NNN) linked to a universal left sample index sequence and/or the right sample index comprises a short random sequence (e.g., NNN) linked to a right universal sample index sequence. The at least one sample index sequence can include sequence diversity for improved base calling. The at least one sample index sequence can be used to improve base calling accuracy.

In some embodiments, the short random sequence (e.g., NNN) is positioned upstream of the universal sample index sequence so that during a sequencing run the random sequence portion is sequenced before the universal sample index sequence. In some embodiments, the short random sequence is positioned downstream of the universal sample index sequence so that during a sequencing run the random portion is sequenced after the universal sample index sequence.

In some embodiments, in the random sequence each base “N” at a given position is independently selected from A, G, C, T or U. In some embodiments, the random sequence lacks consecutive repeat sequences having 2 or 3 of the same nucleo-base, for example AA, TT, CC, GG, UU, AAA, TTT, CCC, GGG or UUU. In some embodiments, in a population of library molecules the universal sample index sequences include a short random sequence having a high diversity sequence which includes approximately equal proportions of all four nucleotides (e.g., A, G, C, T and/or U) that will be represented in each cycle of a sequencing run.

In some embodiments, the short random sequence (e.g., NNN) comprises 3-20 nucleotides, or 3-10 nucleotides, or 3-8 nucleotides, or 3-6 nucleotides, or 3-5 nucleotides, or 3-4 nucleotides.

In some embodiments, the short random sequence (e.g., NNN) includes, but is not limited to, AGC, AGT, GAC, GAT, CAT, CAG, TAG, TAC. The skilled artisan will recognize that many more random sequences can be prepared (e.g., 64 possible combinations) where each base “N” at a given position in the random sequence is independently selected from A, G, C, T or U.

In some embodiments, the universal sample index sequence comprises 5-20 nucleotides, or 7-18 nucleotides, or 9-16 nucleotides.

Exemplary sample index sequence that include a short random sequence NNN linked directly to a universal sample index sequence include but are not limited to: NNNGTAGGAGCC; NNNCCGCTGCTA; NNNAACAACAAG; NNNGGTGGTCTA; NNNTTGGCCAAC; NNNCAGGAGTGC; and NNNATCACACTA. The skilled artisan will recognize that the universal sample index can be any length and have any sequence that can be used to distinguish sequences of interest obtained from different sample sources in a multiplex assay. In a population of a given sample index, for example and without limitation NNNGTAGGAGCC, the population contains a mixture of individual sample index molecules each carrying the same universal sample index sequence (e.g., GTAGGAGCC) and a different short random sequence (e.g., NNN), where up to 64 different short random sequences may be present in the population of the given sample index.

In some embodiments, at least one library molecule comprises a right sample index comprising a short random sequence (e.g., NNN) directly linked to a right universal sample index sequence. In some embodiments, the at least one library molecule further comprises a left sample index comprising a left universal sample index sequence that differs from the right universal sample index sequence.

In some embodiments, the random sequence (e.g., NNN) provides a balanced ratio of nucleo-bases adenine, cytosine, guanine, thymine and/or uracil. In some embodiments, in a population of sample-indexed library molecules, the random sequence (e.g., NNN) together with at least a portion of the universal sample index sequence provide a balanced ratio of nucleo-bases adenine, cytosine, guanine, thymine and/or uracil represented in each cycle of a sequencing run.

In some embodiments, a sequencing reaction includes use of polymerases and nucleotides (e.g., nucleotide analogs) that are labeled with a different fluorophore that corresponds to the nucleo-base. In some embodiments, sequencing the random sequence (e.g., NNN) using labeled nucleotides provides a balanced ratio of fluorescent colors that correspond to the nucleo-bases adenine, cytosine, guanine, thymine and/or uracil in each cycle of a sequencing run. In some embodiments, sequencing the random sequence (e.g., NNN) and at least a portion of the universal sample index sequence using labeled nucleotides provides a balanced ratio of fluorescent colors that correspond to nucleo-bases adenine, cytosine, guanine, thymine and/or uracil. The labeled nucleotides can emit fluorescent signals during the sequencing reactions. In some embodiments, the sequencing reaction is conducted on a sequencing apparatus having a detector that captures fluorescent images from sequencing reactions on the immobilized template molecules. The sequencing apparatus can be configured to relay the fluorescent imaging data captured by the detector to a computer system that is programmed to determine the location (e.g., mapping) of the immobilized template molecules on the flowcell. The computer system can generate a map of the locations of the immobilized template molecules based on the fluorescent imaging data of only the random sequence (e.g., NNN), or based on the random sequence (e.g., NNN) and at least a portion the universal sample index sequence. Thus, the few numbers of sequencing cycles used to sequence the random sequence (e.g., NNN) and optionally a portion of the universal sample index sequence can be used to generate a map of the location of the immobilized template molecules. The computer system can be configured to extract the fluorescent color and intensity of only the random sequence (e.g., NNN), or the random sequence (e.g., NNN) and at least a portion of the universal sample index sequence. The computer system can be configured to use the location of a given immobilized template molecule and the fluorescent color and intensity associated with the given template molecule (which were established while sequencing the random sequence) for base calling while sequencing the insert region (e.g., sequence-of-interest). The computer system can be configured to detect phasing and pre-phasing while sequencing the random sequence (e.g., NNN) and the universal sample index sequence, and the insert region. In some embodiments, the balanced ratio of fluorescent colors provided by the random sequence (e.g., NNN) at each sequencing cycle can improve the quality of the data which is processed from the fluorescent images captured by the detector, and can in turn improve the capability by the computer system to determine the location of the immobilized template molecules on the flowcell, and the color and intensity, all of which can improve base calling accuracy and quality scores of the sequenced insert region.

In some embodiments, a sequencing reaction includes use of polymerases and multivalent molecules that are labeled with a different fluorophore that corresponds to the nucleo-base (e.g., adenine, guanine, cytosine, thymine or uracil) of the nucleotide units that are attached to the nucleotide arms in a given multivalent molecule. In some embodiments, the core of individual multivalent molecules is attached to a fluorophore which corresponds to the nucleotide units (e.g., adenine, guanine, cytosine, thymine or uracil) that are attached to the nucleotide arms in a given multivalent molecule. In some embodiments, at least one of the nucleotide arms of the multivalent molecule comprises a linker and/or nucleotide base that is attached to a fluorophore, and wherein the fluorophore which is attached to a given linker or nucleotide base corresponds to the nucleotide base (e.g., adenine, guanine, cytosine, thymine or uracil) of the nucleotide arm. In some embodiments, sequencing the random sequence (e.g., NNN) using labeled multivalent molecules provides a balanced ratio of fluorescent colors that correspond to the nucleo-bases adenine, cytosine, guanine, thymine and/or uracil in each cycle of a sequencing run. In some embodiments, sequencing the random sequence (e.g., NNN) and at least a portion of the universal sample index sequence using labeled multivalent molecules provides a balanced ratio of fluorescent colors that correspond to nucleo-bases adenine, cytosine, guanine, thymine and/or uracil. The labeled multivalent molecules emit fluorescent signals during the sequencing reactions. In some embodiments, the sequencing reaction is conducted on a sequencing apparatus having a detector that captures fluorescent images from sequencing reactions on the immobilized template molecules. The sequencing apparatus can be configured to relay the fluorescent imaging data captured by the detector to a computer system that is programmed to determine the location (e.g., mapping) of the immobilized template molecules (polonies) on the flowcell. The computer system can generate a map of the locations of the immobilized template molecules based on the fluorescent imaging data of only the random sequence (e.g. NNN), or based on the random sequence (e.g., NNN) and at least a portion of the universal sample index sequence. Thus, the few numbers of sequencing cycles used to sequence the random sequence (e.g., NNN) and optionally a portion of the universal sample index sequence can be used to generate a map of the location of the immobilized template molecules. The computer system can be configured to extract the fluorescent color and intensity of only the random sequence (e.g., NNN) or the random sequence (e.g., NNN) and the universal sample index sequence. The computer system can be configured to use the location of a given immobilized template molecule and the fluorescent color and intensity associated with the given template molecule (which were established while sequencing the random sequence) for base calling while sequencing the insert region. The computer system can be configured to detect phasing and pre-phasing while sequencing the random sequence (e.g., NNN) and the universal sample index sequence, and the insert region. In some embodiments, the balanced ratio of fluorescent colors provided by the random sequence (e.g., NNN) at each sequencing cycle can improve the quality of the data which is processed from the fluorescent images captured by the detector, and can in turn improve the capability by the computer system to determine the location of the immobilized template molecules on the flowcell, and the color and intensity, all of which can improve base calling accuracy and quality scores of the sequenced insert region.

### A First Embodiment: Order of Sequencing Sample Index Sequences

In some embodiments, the order of sequencing comprises: (1) sequencing the right sample index where the right index comprises a first random sequence (e.g., NNN) and a right universal sample index sequence; (2) sequencing the left sample index; and (3) sequencing the insert region. In some embodiments, the left sample index comprises a left universal sample index sequence. In some embodiments, the left sample index comprises a second random sequence (e.g., NNN) and a left universal sample index sequence. In some embodiments, sequencing the right sample index region, including the first random sequence (e.g., NNN) and right universal sample index sequence, may provide enough nucleotide diversity so that sequencing the left sample index can be omitted.

In some embodiments, methods for sequencing the template molecules immobilized to a support, wherein individual template molecules comprise: (i) a universal binding sequence for a first surface primer, (ii) a left sample index sequence having a universal left sample index sequence, (iii) a universal binding sequence for a forward sequencing primer, (iv) a sequence of interest, (v) a universal binding sequence for a reverse sequencing primer, (vi) a right sample index sequence having a short random sequence (e.g. NNN) linked directly to a right universal sample index sequence, and (vii) a universal binding sequence for a second surface primer (e.g., see FIG. 1), wherein the method comprises step (a): hybridizing the template molecules with a first plurality of soluble sequencing primers that hybridize to the universal binding sequence for a reverse sequencing primer and sequencing the right sample index sequence including sequencing the short random sequence (e.g., NNN) and the right universal sample index sequence thereby generating a first plurality of sample index extension products that are hybridized to the immobilized template molecules, wherein the first plurality of sample index extension products are complementary to the right sample index sequence having a short random sequence (e.g., NNN) linked directly to a right universal sample index sequence.

In some embodiments, the methods for sequencing further comprise step (b): removing the first plurality of sample index extension products and retaining the immobilized template molecules.

In some embodiments, the methods for sequencing further comprise step (c): hybridizing the retained immobilized template molecules with a second plurality of soluble sequencing primers that hybridize to the universal binding sequence for the first surface primer and sequencing the left sample index sequence thereby generating a second plurality of sample index extension products that are hybridized to the immobilized template molecules, wherein the second plurality of sample index extension products are complementary to the left sample index sequence having a left universal sample index sequence.

In some embodiments, the methods for sequencing further comprise step (d): removing the second plurality of sample index extension products and retaining the immobilized template molecules.

In some embodiments, the methods for sequencing further comprise step (e): hybridizing the retained immobilized template molecules with a third plurality of soluble sequencing primers that hybridize to the universal binding sequence for the forward sequencing primer and sequencing the insert region thereby generating a plurality of insert extension products that are hybridized to the immobilized template molecules, wherein the plurality of insert extension products are complementary to the sequence of interest.

In some embodiments, the methods for sequencing further comprise step (f1): assigning the sequence of (i) the insert region to (ii) the right sample index sequence having a short random sequence (e.g., NNN) linked directly to a right universal sample index sequence, thereby identifying the insert region as being obtained from a first source.

In some embodiments, the methods for sequencing further comprise step (f2): assigning the sequence of (i) the insert region to (ii) the right sample index sequence having a short random sequence (e.g., NNN) linked directly to a right universal sample index sequence, and (iii) the left sample index sequence, thereby identifying the insert region as being obtained from a first source.

In some embodiments, the removing of the plurality of sequencing extension products of steps (b) and (d) can be conducted using a denaturation reagent comprising SSC (e.g., saline-sodium citrate) buffer with or without formamide, at a temperature that promotes nucleic acid denaturation. In some embodiments, the temperature is between 50-90° C., or any range therebetween.

In some embodiments, the removing of the plurality of sequencing extension products of steps (b) and (d) can be conducted using an enzyme-base reagent comprising a double-stranded DNA specific exonuclease having 5′ to 3′ degradation activity (e.g., T7 exonuclease encoded by T7 phage gene 6 or lambda exonuclease), at least one aqueous solvent, a pH buffering agent, a monovalent salt, a divalent salt, and enzyme stabilizer (e.g., BSA). In some embodiments, the aqueous solvent comprises water. In some embodiments, the enzyme-based reagent further comprises NaCl at a concentration suitable to reduce 5′ flap endonuclease activity of the T7 exonuclease. The enzyme-based reagent can include NaCl at a concentration of about 20-150 mM, or about 20-40 mM, or about 40-60 mM, or about 60-80 mM, or about 80-100 mM, or about 100-120 mM, or about 120-150 mM, or at any range therebetween.

In some embodiments, the sequencing of steps (a), (c) and (e) include conducting any of the sequencing methods described herein that employ sequencing polymerases and detectably labeled nucleotide analogs. In some embodiments, the sequencing of steps (a), (c) and (e) include conducting any of the two-stage sequencing methods described herein that employ sequencing polymerases, detectably labeled multivalent molecules, and nucleotide analogs. In some embodiments, the sequencing of steps (a), (c) and (e) include conducting any of the sequencing-by-binding methods described herein.

In some embodiments, the density of the plurality of template molecules immobilized to the support is about 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) per mm². In some embodiments, the plurality of template molecules is immobilized at random locations on the support. In some embodiments, the plurality of template molecules is immobilized on the support in a predetermined pattern.

### A Second Embodiment: Order of Sequencing Sample Index Sequences

In some embodiments, the order of sequencing comprises: (1) sequencing the right sample index where the right index comprises a random sequence (e.g., NNN) and a universal sample index sequence; (2) sequencing the insert region; and (3) sequencing the left sample index. In some embodiments, the left sample index comprises a left universal sample index sequence. In some embodiments, the left sample index comprises a second random sequence (e.g., NNN) and a left universal sample index sequence. In some embodiments, sequencing the right sample index region, including the first random sequence (e.g., NNN) and right universal sample index sequence, may provide enough nucleotide diversity so that sequencing the left sample index can be omitted.

In some embodiments, methods for sequencing the template molecules immobilized to a support, wherein individual template molecules comprise: (i) a universal binding sequence for a first surface primer, (ii) a left sample index sequence having a left universal sample index sequence (iii) a universal binding sequence for a forward sequencing primer, (iv) a sequence of interest, (v) a universal binding sequence for a reverse sequencing primer, (vi) a right sample index sequence having a short random sequence (e.g., NNN) linked directly to a right universal sample index sequence, and (vii) a universal binding sequence for a second surface primer (e.g., see FIG. 1), wherein the method comprises step (a): hybridizing the template molecules with a first plurality of soluble sequencing primers that hybridize to the universal binding sequence for a reverse sequencing primer and sequencing the right sample index sequence including sequencing the short random sequence (e.g., NNN) and the right universal sample index sequence thereby generating a first plurality of sample index extension products that are hybridized to the immobilized template molecules, wherein the first plurality of sample index extension products are complementary to the right sample index sequence having a short random sequence (e.g., NNN) linked directly to a right universal sample index sequence.

In some embodiments, the methods for sequencing further comprise step (b): removing the first plurality of sample index extension products and retaining the immobilized template molecules.

In some embodiments, the methods for sequencing further comprise step (c): hybridizing the retained immobilized template molecules with a second plurality of soluble sequencing primers that hybridize to the universal binding sequence for a forward sequencing primer and sequencing the insert region thereby generating a plurality of insert extension products that are hybridized to the immobilized template molecules, wherein the plurality of insert extension products are complementary to the sequence of interest.

In some embodiments, the methods for sequencing further comprise step (d): removing the plurality of insert extension products and retaining the immobilized template molecules.

In some embodiments, the methods for sequencing further comprise step (e): hybridizing the retained immobilized template molecules with a third plurality of soluble sequencing primers that hybridize to the universal binding sequence for a first surface primer and sequencing the left sample index sequence thereby generating a second plurality of sample index extension products that are hybridized to the immobilized template molecules, wherein the second plurality of sample index extension products are complementary to the left sample index sequence having a left universal sample index sequence.

In some embodiments, the methods for sequencing further comprise step (f1): assigning the sequence of (i) the insert region to (ii) the right sample index sequence having a short random sequence (e.g., NNN) linked directly to a right universal sample index sequence, thereby identifying the insert region as being obtained from a first source.

In some embodiments, the methods for sequencing further comprise step (f2): assigning the sequence of (i) the insert region to (ii) the right sample index sequence having a short random sequence (e.g., NNN) linked directly to a right universal sample index sequence, and (iii) the left sample index sequence, thereby identifying the insert region as being obtained from a first source.

In some embodiments, the removing of the plurality of sequencing extension products of steps (b) and (d) can be conducted using a denaturation reagent comprising SSC (e.g., saline-sodium citrate) buffer with or without formamide, at a temperature that promotes nucleic acid denaturation. In some embodiments, the temperature is 50-90° C., or any range therebetween.

In some embodiments, the removing of the plurality of sequencing extension products of steps (b) and (d) can be conducted using an enzyme-base reagent comprising a double-stranded DNA specific exonuclease having 5′ to 3′ degradation activity (e.g., T7 exonuclease encoded by T7 phage gene 6 or lambda exonuclease), at least one aqueous solvent, a pH buffering agent, a monovalent salt, a divalent salt, and enzyme stabilizer (e.g., BSA). In some embodiments, the aqueous solvent comprises water. In some embodiments, the enzyme-based reagent further comprises NaCl at a concentration suitable to reduce 5′ flap endonuclease activity of the T7 exonuclease. The enzyme-based reagent can include NaCl at a concentration of about 20-150 mM, or about 20-40 mM, or about 40-60 mM, or about 60-80 mM, or about 80-100 mM, or about 100-120 mM, or about 120-150 mM, or any range therebetween.

In some embodiments, the sequencing of steps (a), (c) and (e) include conducting any of the sequencing methods described herein that employ sequencing polymerases and detectably labeled nucleotide analogs. In some embodiments, the sequencing of steps (a), (c) and (e) include conducting any of the two-stage sequencing methods described herein that employ sequencing polymerases, detectably labeled multivalent molecules, and nucleotide analogs. In some embodiments, the sequencing of steps (a), (c) and (e) include conducting any of the sequencing-by-binding methods described herein.

In some embodiments, the density of the plurality of template molecules immobilized to the support is about 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) per mm². In some embodiments, the plurality of template molecules is immobilized at random locations on the support. In some embodiments, the plurality of template molecules is immobilized on the support in a predetermined pattern.

### A Third Embodiment: Order of Sequencing Sample Index Sequences

In some embodiments, the order of sequencing comprises: (1) sequencing the insert region; (2) sequencing the right sample index where the right index comprises a random sequence (e.g., NNN) and a universal sample index sequence; and (3) sequencing the left sample index. In some embodiments, the left sample index comprises a left universal sample index sequence. In some embodiments, the left sample index comprises a second random sequence (e.g., NNN) and a left universal sample index sequence. In some embodiments, sequencing the right sample index region, including the first random sequence (e.g., NNN) and right universal sample index sequence, may provide enough nucleotide diversity so that sequencing the left sample index can be omitted.

In some embodiments, methods for sequencing the template molecules immobilized to a support, wherein individual template molecules comprise: (i) a universal binding sequence for a first surface primer, (ii) a left sample index sequence having a left universal sample index sequence (iii) a universal binding sequence for a forward sequencing primer, (iv) a sequence of interest, (v) a universal binding sequence for a reverse sequencing primer, (vi) a right sample index sequence having a short random sequence (e.g., NNN) linked directly to a right universal sample index sequence, and (vii) a universal binding sequence for a second surface primer (e.g., see FIG. 1), wherein the method comprises step (a): hybridizing the template molecules with a first plurality of soluble sequencing primers that hybridize to the universal binding sequence for a forward sequencing primer and sequencing the insert region thereby generating a plurality of insert extension products that are hybridized to the immobilized template molecules, wherein the plurality of insert extension products are complementary to the sequence of interest.

In some embodiments, the methods for sequencing further comprise step (b): removing the plurality of insert extension products and retaining the immobilized template molecules.

In some embodiments, the methods for sequencing further comprise step (c): hybridizing the template molecules with a second plurality of soluble sequencing primers that hybridize to the universal binding sequence for a reverse sequencing primer and sequencing the right sample index sequence including sequencing the short random sequence (e.g., NNN) and the right universal sample index sequence thereby generating a first plurality of sample index extension products that are hybridized to the immobilized template molecules, wherein the first plurality of sample index extension products are complementary to the right sample index sequence having a short random sequence (e.g., NNN) linked directly to a right universal sample index sequence.

In some embodiments, the methods for sequencing further comprise step (d): removing the first plurality of sample index extension products and retaining the immobilized template molecules.

In some embodiments, the methods for sequencing further comprise step (e): hybridizing the retained immobilized template molecules with a third plurality of soluble sequencing primers that hybridize to the universal binding sequence for a first surface primer and sequencing the left sample index sequence thereby generating a second plurality of sample index extension products that are hybridized to the immobilized template molecules, wherein the second plurality of sample index extension products are complementary to the left sample index sequence having a left universal sample index sequence.

In some embodiments, the methods for sequencing further comprise step (f1): assigning the sequence of (i) the insert region to (ii) the right sample index sequence having a short random sequence (e.g., NNN) linked directly to a right universal sample index sequence, thereby identifying the insert region as being obtained from a first source.

In some embodiments, the methods for sequencing further comprise step (f2): assigning the sequence of (i) the insert region to (ii) the right sample index sequence having a short random sequence (e.g., NNN) linked directly to a right universal sample index sequence, and (iii) the left sample index sequence, thereby identifying the insert region as being obtained from a first source.

In some embodiments, the removing of the plurality of sequencing extension products of steps (b) and (d) can be conducted using a denaturation reagent comprising SSC (e.g., saline-sodium citrate) buffer with or without formamide, at a temperature that promotes nucleic acid denaturation. In some embodiments, the temperature is 50-90° C., or any range therebetween.

In some embodiments, the removing of the plurality of sequencing extension products of steps (b) and (d) can be conducted using an enzyme-base reagent comprising a double-stranded DNA specific exonuclease having 5′ to 3′ degradation activity (e.g., T7 exonuclease encoded by T7 phage gene 6 or lambda exonuclease), at least one aqueous solvent, a pH buffering agent, a monovalent salt, a divalent salt, and enzyme stabilizer (e.g., BSA). In some embodiments, the aqueous solvent comprises water. In some embodiments, the enzyme-based reagent further comprises NaCl at a concentration suitable to reduce 5′ flap endonuclease activity of the T7 exonuclease. The enzyme-based reagent can include NaCl at a concentration of about 20-150 mM, or about 20-40 mM, or about 40-60 mM, or about 60-80 mM, or about 80-100 mM, or about 100-120 mM, or about 120-150 mM, or any range therebetween.

In some embodiments, the sequencing of steps (a), (c) and (e) include conducting any of the sequencing methods described herein that employ sequencing polymerases and detectably labeled nucleotide analogs. In some embodiments, the sequencing of steps (a), (c) and (e) include conducting any of the two-stage sequencing methods described herein that employ sequencing polymerases, detectably labeled multivalent molecules, and nucleotide analogs. In some embodiments, the sequencing of steps (a), (c) and (e) include conducting any of the sequencing-by-binding methods described herein.

In some embodiments, the density of the plurality of template molecules immobilized to the support is about 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) per mm². In some embodiments, the plurality of template molecules is immobilized at random locations on the support. In some embodiments, the plurality of template molecules is immobilized on the support in a predetermined pattern.

### A Fourth Embodiment: Order of Sequencing

In some embodiments, the order of sequencing comprises: (1) sequencing the first 3-5 bases of the insert region; (2) sequencing the right sample index where the right index comprises an optional random sequence (e.g., NNN) and a universal sample index sequence; and (3) sequencing the left sample index. In some embodiments, the left sample index comprises a left universal sample index sequence. In some embodiments, the left sample index comprises a second random sequence (e.g., NNN) and a left universal sample index sequence. In some embodiments, sequencing the first 3-5 bases of the insert region may provide enough sequence diversity so that the right sample index and the left sample index do not include a short random sequence (e.g., NNN). In some embodiments, sequencing the right sample index region, including the first random sequence (e.g., NNN) and right universal sample index sequence, may provide enough nucleotide diversity so that sequencing the left sample index can be omitted.

In some embodiments, methods for sequencing the template molecules immobilized to a support, wherein individual template molecules comprise: (i) a universal binding sequence for a first surface primer, (ii) a left sample index sequence having a left universal sample index sequence (iii) a universal binding sequence for a forward sequencing primer, (iv) a sequence of interest, (v) a universal binding sequence for a reverse sequencing primer, (vi) a right sample index sequence having a short random sequence (e.g., NNN) linked directly to a right universal sample index sequence, and (vii) a universal binding sequence for a second surface primer (e.g., see FIG. 1), wherein the method comprises step (a): hybridizing the template molecules with a first plurality of soluble sequencing primers that hybridize to the universal binding sequence for a forward sequencing primer and sequencing the first 3-5 bases of the insert region thereby generating a plurality of insert extension products that are hybridized to the immobilized template molecules, wherein the plurality of insert extension products are complementary to the sequence of interest. The sequence of the first 3-5 bases of the insert region may provide sufficient sequence diversity and color balance for polony mapping and template registration.

In some embodiments, the methods for sequencing further comprise step (b): removing the plurality of insert extension products and retaining the immobilized template molecules.

In some embodiments, the methods for sequencing further comprise step (c): hybridizing the template molecules with a second plurality of soluble sequencing primers that hybridize to the universal binding sequence for a reverse sequencing primer and sequencing the right sample index sequence including sequencing the short random sequence (e.g., NNN) if present and the right universal sample index sequence thereby generating a first plurality of sample index extension products that are hybridized to the immobilized template molecules, wherein the first plurality of sample index extension products are complementary to the right sample index sequence.

In some embodiments, the methods for sequencing further comprise step (d): removing the first plurality of sample index extension products and retaining the immobilized template molecules.

In some embodiments, the methods for sequencing further comprise step (e): hybridizing the retained immobilized template molecules with a third plurality of soluble sequencing primers that hybridize to the universal binding sequence for a first surface primer and sequencing the left sample index sequence thereby generating a second plurality of sample index extension products that are hybridized to the immobilized template molecules, wherein the second plurality of sample index extension products are complementary to the left sample index sequence having a left universal sample index sequence.

In some embodiments, the methods for sequencing further comprise step (f): removing the second plurality of sample index extension products and retaining the immobilized template molecules.

In some embodiments, the methods for sequencing further comprise step (g): hybridizing the template molecules with a fourth plurality of soluble sequencing primers that hybridize to the universal binding sequence for a forward sequencing primer and sequencing the full length of the insert region thereby generating a plurality of full length insert extension products that are hybridized to the immobilized template molecules, wherein the plurality of full length insert extension products are complementary to the sequence of interest.

In some embodiments, the methods for sequencing further comprise step (h1): assigning the full-length sequence of (i) the insert region to (ii) the right sample index sequence, thereby identifying the insert region as being obtained from a first source.

In some embodiments, the methods for sequencing further comprise step (h2): assigning the full-length sequence of (i) the insert region to (ii) the right sample index sequence, and (iii) the left sample index sequence, thereby identifying the insert region as being obtained from a first source.

In some embodiments, the removing of the plurality of sequencing extension products of steps (b), (d) and (f) can be conducted using a denaturation reagent comprising SSC (e.g., saline-sodium citrate) buffer with or without formamide, at a temperature that promotes nucleic acid denaturation. In some embodiments, the temperature is 50-90° C., or any range therebetween.

In some embodiments, the removing of the plurality of sequencing extension products of steps (b) and (d) can be conducted using an enzyme-base reagent comprising a double-stranded DNA specific exonuclease having 5′ to 3′ degradation activity (e.g., T7 exonuclease encoded by T7 phage gene 6 or lambda exonuclease), at least one aqueous solvent, a pH buffering agent, a monovalent salt, a divalent salt, and enzyme stabilizer (e.g., BSA). In some embodiments, the aqueous solvent comprises water. In some embodiments, the enzyme-based reagent further comprises NaCl at a concentration suitable to reduce 5′ flap endonuclease activity of the T7 exonuclease. The enzyme-based reagent can include NaCl at a concentration of about 20-150 mM, or about 20-40 mM, or about 40-60 mM, or about 60-80 mM, or about 80-100 mM, or about 100-120 mM, or about 120-150 mM, or any range therebetween.

In some embodiments, the sequencing of steps (a), (c), (e) and (g) include conducting any of the sequencing methods described herein that employ sequencing polymerases and detectably labeled nucleotide analogs. In some embodiments, the sequencing of steps (a), (c), (e) and (g) include conducting any of the two-stage sequencing methods described herein that employ sequencing polymerases, detectably labeled multivalent molecules, and nucleotide analogs. In some embodiments, the sequencing of steps (a), (c), (e) and (g) include conducting any of the sequencing-by-binding methods described herein.

In some embodiments, the density of the plurality of template molecules immobilized to the support is about 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) per mm². In some embodiments, the plurality of template molecules is immobilized at random locations on the support. In some embodiments, the plurality of template molecules is immobilized on the support in a predetermined pattern.

### A Fifth Embodiment: Order of Sequencing

In some embodiments, the order of sequencing comprises: (1) sequencing the first 3-5 bases of the insert region of the immobilized template molecule (e.g., sequencing in a forward direction); (2) sequencing the right sample index where the right index comprises a first random sequence (e.g., NNN) and a right universal sample index sequence; (3) sequencing the left sample index; (4) conducting a pairwise turn reaction so that the immobilized template molecule is replaced with an immobilized strand that is complementary to the template molecule; and (5) sequencing the full-length of the insert region of the immobilized complementary strand (e.g., sequencing in the reverse direction). In some embodiments, the sequences of the first 3-5 bases of the insert region of a population of library molecules may provide enough sequence diversity for improved base-calling accuracy. In some embodiments, the left sample index comprises a left universal sample index sequence. In some embodiments, the left sample index comprises a second random sequence (e.g., NNN) and a left universal sample index sequence. In some embodiments, sequencing the right sample index region, including the first random sequence (e.g., NNN) and right universal sample index sequence, may provide enough nucleotide diversity so that sequencing the left sample index can be omitted.

In some embodiments, methods for sequencing the template molecules immobilized to a support, wherein individual template molecules are covalently linked to an immobilized capture primer that lacks uracil bases, and individual template molecules comprise randomly-distributed uracil bases, and individual template molecules comprise: (i) a universal binding sequence for a first surface primer, (ii) a left sample index sequence having a left universal sample index sequence (iii) a universal binding sequence for a forward sequencing primer, (iv) a sequence of interest, (v) a universal binding sequence for a reverse sequencing primer, (vi) a right sample index sequence having a short random sequence (e.g. NNN) linked directly to a right universal sample index sequence, and (vii) a universal binding sequence for a second surface primer (e.g., see FIG. 1), wherein the method comprises step (a): hybridizing the template molecules with a first plurality of soluble sequencing primers (e.g., forward sequencing primers) that hybridize to the universal binding sequence for a forward sequencing primer and sequencing the first 3-5 bases of the insert region thereby generating a plurality of insert extension products that are hybridized to the immobilized template molecules, wherein the plurality of insert extension products are complementary to the sequence of interest.

In some embodiments, the methods for sequencing further comprise step (b): removing the plurality of insert extension products and retaining the immobilized template molecules.

In some embodiments, the methods for sequencing further comprise step (c): hybridizing the template molecules with a second plurality of soluble sequencing primers that hybridize to the universal binding sequence for a reverse sequencing primer and sequencing the right sample index sequence including sequencing the short random sequence (e.g., NNN) and the right universal sample index sequence thereby generating a first plurality of sample index extension products that are hybridized to the immobilized template molecules, wherein the first plurality of sample index extension products are complementary to the right sample index sequence having a short random sequence (e.g., NNN) linked directly to a right universal sample index sequence.

In some embodiments, the methods for sequencing further comprise step (d): removing the first plurality of sample index extension products and retaining the immobilized template molecules.

In some embodiments, the methods for sequencing further comprise step (e): hybridizing the retained immobilized template molecules with a third plurality of soluble sequencing primers that hybridize to the universal binding sequence for a first surface primer and sequencing the left sample index sequence thereby generating a second plurality of sample index extension products that are hybridized to the immobilized template molecules, wherein the second plurality of sample index extension products are complementary to the left sample index sequence having a left universal sample index sequence.

In some embodiments, the methods for sequencing further comprise step (f): replacing the second plurality of sample index extension products that are hybridized to the immobilized template molecules by conducting a primer extension reaction using strand-displacing polymerases and a plurality of nucleotides to generate an extension product that is hybridized to the immobilized template molecules including the immobilized capture primer.

In some embodiments, the methods for sequencing further comprise step (g): removing the immobilized template molecules by generating abasic sites in the immobilized template molecules at the uracil sites and generating gaps at the abasic sites thereby generating gap-containing template molecules while retaining the extension products that was generated in step (f) where individual extension products are retained by being hybridized to an immobilized capture primer. In some embodiments, pairwise turn is achieved by conducting steps (g) and (h).

In some embodiments, the methods for sequencing further comprise step (h): hybridizing the retained extension products with a fourth plurality of soluble sequencing primers (e.g., reverse sequencing primers) that hybridize to universal binding sequence for a reverse sequencing primer and sequencing the insert region (e.g., sequencing at least a portion or the full length of the insert region).

In some embodiments, the methods for sequencing further comprise step (i1): assigning the sequence of (i) the insert region to (ii) the right sample index sequence having a short random sequence (e.g., NNN) linked directly to a right universal sample index sequence, thereby identifying the insert region as being obtained from a first source.

In some embodiments, the methods for sequencing further comprise step (i2): assigning the sequence of (i) the insert region to (ii) the right sample index sequence having a short random sequence (e.g., NNN) linked directly to a right universal sample index sequence, and (iii) the left sample index sequence, thereby identifying the insert region as being obtained from a first source.

In some embodiments, the removing of the plurality of sequencing extension products of steps (b) and (d) can be conducted using a denaturation reagent comprising SSC (e.g., saline-sodium citrate) buffer with or without formamide, at a temperature that promotes nucleic acid denaturation such. In some embodiments, the temperature is 50-90° C., or any range therebetween.

In some embodiments, the removing of the plurality of sequencing extension products of steps (b) and (d) can be conducted using an enzyme-base reagent comprising a double-stranded DNA specific exonuclease having 5′ to 3′ degradation activity (e.g., T7 exonuclease encoded by T7 phage gene 6 or lambda exonuclease), at least one aqueous solvent, a pH buffering agent, a monovalent salt, a divalent salt, and enzyme stabilizer (e.g., BSA). In some embodiments, the aqueous solvent comprises water. In some embodiments, the enzyme-based reagent further comprises NaCl at a concentration suitable to reduce 5′ flap endonuclease activity of the T7 exonuclease. The enzyme-based reagent can include NaCl at a concentration of about 20-150 mM, or about 20-40 mM, or about 40-60 mM, or about 60-80 mM, or about 80-100 mM, or about 100-120 mM, or about 120-150 mM, or any range therebetween.

In some embodiments, the sequencing of steps (a), (c), (e) and (h) include conducting any of the sequencing methods described herein that employ sequencing polymerases and detectably labeled nucleotide analogs. In some embodiments, the sequencing of steps (a), (c), (e) and (h) include conducting any of the two-stage sequencing methods described herein that employ sequencing polymerases, detectably labeled multivalent molecules, and nucleotide analogs. In some embodiments, the sequencing of steps (a), (c), (e) and (h) include conducting any of the sequencing-by-binding methods described herein.

In some embodiments, the density of the plurality of template molecules immobilized to the support is about 10²-10¹⁵ (e.g., 10², 10³, 10⁴, 10¹, 10⁶, 10⁷, 10⁸, 10⁹, 10¹⁰, 10¹¹, 10¹², 10¹³, 10¹⁴, or 10¹⁵) per mm². In some embodiments, the plurality of template molecules is immobilized at random locations on the support. In some embodiments, the plurality of template molecules is immobilized on the support in a predetermined pattern.

Supports with Low Non-Specific Binding Coatings

In some aspects, the present disclosure provides compositions and methods for use of a support having a plurality of surface primers immobilized thereon, for preparing any of the immobilized concatemers described herein. In some embodiments, the support is passivated with a low non-specific binding coating (e.g., FIG. 16). The surface coatings described herein can exhibit very low non-specific binding to reagents typically used for nucleic acid capture, amplification and sequencing workflows, such as dyes, nucleotides, enzymes, and nucleic acid primers. The surface coatings can exhibit low background fluorescence signals or high contrast-to-noise (CNR) ratios compared to conventional surface coatings.

In some embodiments, the supports comprise a substrate (or support structure), one or more layers of a covalently or non-covalently attached low-binding, chemical modification layers, e.g., silane layers, polymer films, and one or more covalently or non-covalently attached primer sequences that may be used for tethering single-stranded target nucleic acid(s) to the support surface. In some embodiments, the formulation of the surface, e.g., the chemical composition of one or more layers, the coupling chemistry used to cross-link the one or more layers to the support surface and/or to each other, and the total number of layers, may be varied such that non-specific binding of proteins, nucleic acid molecules, and other hybridization and amplification reaction components to the support surface is minimized or reduced relative to a comparable monolayer. In some embodiments, the formulation of the surface may be varied such that non-specific hybridization on the support surface is minimized or reduced relative to a comparable monolayer. The formulation of the surface may be varied such that non-specific amplification on the support surface is minimized or reduced relative to a comparable monolayer. The formulation of the surface may be varied such that specific amplification rates and/or yields on the support surface are maximized. Amplification levels suitable for detection can be achieved in no more than 2, 3, 4, 5, 6, 7, 8, 9, 10, 15, 20, 25, 30, or more than 30 amplification cycles in some embodiments disclosed herein.

The substrate or support structure that comprises the one or more chemically-modified layers, e.g., layers of a low non-specific binding polymer, may be independent or integrated into another structure or assembly. For example, in some embodiments, the substrate or support structure may comprise one or more surfaces within an integrated or assembled microfluidic flow cell. The substrate or support structure may comprise one or more surfaces within a microplate format, e.g., the bottom surface of the wells in a microplate. As noted above, in some embodiments, the substrate or support structure comprises the interior surface (such as the lumen surface) of a capillary. In alternate embodiments the substrate or support structure comprises the interior surface (such as the lumen surface) of a capillary etched into a planar chip.

The attachment chemistry used to graft a first chemically-modified layer to a surface is generally dependent on both the material from which the surface is fabricated and the chemical nature of the layer. In some embodiments, the first layer may be covalently attached to the surface. In some embodiments, the first layer may be non-covalently attached, e.g., adsorbed to the surface through non-covalent interactions such as electrostatic interactions, hydrogen bonding, or van der Waals interactions between the surface and the molecular components of the first layer. In either embodiment, the substrate surface may be treated prior to attachment or deposition of the first layer. Any of a variety of surface preparation techniques known to those of skill in the art may be used to clean or treat the surface. For example, and without limitation, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute one non-limiting approach for covalently modifying the silanol groups on glass or silicon surfaces to attach more reactive functional groups (e.g. amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the surface, where the choice of components used may be varied to alter one or more properties of the surface, e.g., the surface density of functional groups and/or tethered oligonucleotide primers, the hydrophilicity/hydrophobicity of the surface, or the three three-dimensional nature (i.e., “thickness”) of the surface. Examples of suitable polymers that may be used to create one or more layers of low non-specific binding material in any of the disclosed surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the substrate. Alternately, the surface coating may be patterned, such that the chemical modification layers are confined to one or more discrete regions of the substrate. For example, the surface may be patterned using photolithographic techniques to create an ordered array or random pattern of chemically-modified regions on the surface. Alternatively, or in combination, the substrate surface may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In order to achieve low nonspecific binding surfaces, hydrophilic polymers may be nonspecifically adsorbed or covalently grafted to the surface. In some embodiments, passivation is performed utilizing poly(ethylene glycol) (PEG, also known as polyethylene oxide (PEO) or polyoxyethylene) or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting surface. In some embodiments, oligonucleotide primers with different base sequences and base modifications (or other biomolecules, e.g., enzymes or antibodies) may be tethered to the resulting surface layer at various surface densities. In some embodiments, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density. Example of suitable linkers include, without limitation, poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In order to scale primer surface density and add additional dimensionality to hydrophilic or amphoteric surfaces, surfaces comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

As noted, the low non-specific binding coatings of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, in some embodiments, exposure of the surface to fluorescent dyes (e.g., cyanine dyes such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations—provided that care has been taken to ensure that the fluorescence imaging is performed under a condition where fluorescence signal is linearly related (or related in a predictable manner) to the number of fluorophores on the support surface (e.g., under a condition where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some embodiments, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or other molecules) per unit area. In some embodiments, the low-binding supports of the present disclosure may exhibit non-specific protein binding (or non-specific binding of other specified molecules, (e.g., cyanine dyes such as Cy3, or Cy5, etc., fluoresceins, coumarins, rhodamines, etc. or other dyes disclosed herein)) of less than 0.001 molecule per μm2, less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of Cy3 labeled streptavidin (GE Amersham®) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher®), 1 μM Cy5 SA dye (ThermoFisher®), 10 μM Aminoallyl-dUTP-ATTO-647N (Jena Biosciences®), 10 μM Aminoallyl-dUTP-ATTO-Rho11 (Jena Biosciences®), 10 μM Aminoallyl-dUTP-ATTO-Rho11 (Jena Biosciences®), 10 μM 7-Propargylamino-7-deaza-dGTP-Cy5 (Jena Biosciences®, and 10 μM 7-Propargylamino-7-deaza-dGTP-Cy3 (Jena Biosciences®) were incubated on the low binding substrates at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon® instrument using the Cy3, AF555, or Cy5 filter sets (according to dye test performed) as specified by the manufacturer at a PMT gain setting of 800 and resolution of 50-100 μm. For higher resolution imaging, images were collected on an Olympus® IX83 microscope (Olympus Corp.®, Center Valley, PA) with a total internal reflectance fluorescence (TIRF) objective (100×, 1.5 NA, Olympus®), a CCD camera (e.g., an Olympus® EM-CCD monochrome camera, Olympus® XM-10 monochrome camera, or an Olympus® DP80 color and monochrome camera), an illumination source (e.g., an Olympus® 100W Hg lamp, an Olympus® 75W Xe lamp, or an Olympus® U-HGLGPS fluorescence light source), and excitation wavelengths of 532 nm or 635 nm. Dichroic mirrors were purchased from Semrock® (IDEX Health & Science®, LLC, Rochester, New York), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm².

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, in some embodiments adequate wash steps may be performed in less than 30 seconds.

The low-binding surfaces of the present disclosure exhibit significant improvement in stability or durability to prolonged exposure to solvents and elevated temperatures, or to repeated cycles of solvent exposure or changes in temperature. For example, in some embodiments, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100-fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100-fold greater than a signal of an adjacent amplified nucleic acid population region of the surface.

In some embodiments, fluorescence images of the disclosed low background surfaces when used in nucleic acid hybridization or amplification applications to create clusters of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer (e.g., capture primers) may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers on the low binding support surfaces of the present disclosure may range from about 100 primer molecules per μm² to about 100,000 primer molecules per μm². In some embodiments, the resultant surface density of primers on the low binding support surfaces of the present disclosure may range from about 100,000 primer molecules per μm² to about 10¹⁵ primer molecules per μm². In some embodiments, the surface density of primers may be at least 1,000, at least 10,000, at least 100,000, or at least 10¹⁵ primer molecules per μm². In some embodiments, the surface density of primers may be at most 10,000, at most 100,000, at most 1,000,000, or at most 10¹⁵ primer molecules per μm². Any of the lower and upper values described in this paragraph may be combined to form a range included within the present disclosure, for example, in some embodiments the surface density of primers may range from about 10,000 molecules per μm² to about 10¹⁵ molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000 per μm², while also comprising at least a second region having a substantially different local density.

The low non-specific binding coating comprise one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule to about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, and without limitation, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

Fluorescence imaging may be performed using any of a variety of fluorophores, fluorescence imaging techniques, and fluorescence imaging instruments known to those of skill in the art. Examples of suitable fluorescence dyes that may be used (e.g., by conjugation to nucleotides, oligonucleotides, or proteins) include, but are not limited to, fluorescein, rhodamine, coumarin, cyanine, and derivatives thereof, including the cyanine derivatives Cyanine dye-3 (Cy3), Cyanine dye-5 (Cy5), Cyanine dye-7 (Cy7), etc. Examples of fluorescence imaging techniques that may be used include, but are not limited to, fluorescence microscopy imaging, fluorescence confocal imaging, two-photon fluorescence, and the like. Examples of fluorescence imaging instruments that may be used include, but are not limited to, fluorescence microscopes equipped with an image sensor or camera, confocal fluorescence microscopes, two-photon fluorescence microscopes, or custom instruments that comprise a suitable selection of light sources, lenses, mirrors, prisms, dichroic reflectors, apertures, and image sensors or cameras, etc. A non-limiting example of a fluorescence microscope equipped for acquiring images of the disclosed low-binding support surfaces and clonally-amplified colonies (polonies) of template nucleic acid sequences hybridized thereon is the Olympus® IX83 inverted fluorescence microscope equipped with) 20×, 0.75 NA, a 532 nm light source, a bandpass and dichroic mirror filter set optimized for 532 nm long-pass excitation and Cy3 fluorescence emission filter, a Semrock® 532 nm dichroic reflector, and a camera (Andor sCMOS®, Zyla® 4.2) where the excitation light intensity is adjusted to avoid signal saturation. Often, the support surface may be immersed in a buffer (e.g., 25 mM ACES, pH 7.4 buffer) while the image is acquired.

In some instances, the performance of nucleic acid hybridization and/or amplification reactions using the disclosed reaction formulations and low non-specific binding supports may be assessed using fluorescence imaging techniques, where the contrast-to-noise ratio (CNR) of the images provides a key metric in assessing amplification specificity and non-specific binding on the support. CNR is commonly defined as: CNR=(Signal−Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. The surfaces of the instant disclosure are also provided in International Application Serial No. PCT/US2019/061556, which is hereby incorporated by reference in its entirety.

In most ensemble-based sequencing approaches, the background term is typically measured as the signal associated with ‘interstitial’ regions. In addition to “interstitial” background (Bᵢₙₜₑᵣ), “interstitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (S)-Bᵢₙₜₑᵣ in the FOV) yields a discernable feature that can be classified. In some instances, the intrastitial background (Bᵢₙₜᵣₐ) can contribute a confounding fluorescence signal that is not specific to the target of interest, but is present in the same ROI thus making it far more difficult to average and subtract.

The implementation of nucleic acid amplification on the low-binding substrates of the present disclosure may decrease the Bᵢₙₜₑᵣ background signal by reducing non-specific binding, may lead to improvements in specific nucleic acid amplification, and may lead to a decrease in non-specific amplification that can impact the background signal arising from both the interstitial and intrastitial regions. In some instances, the disclosed low-binding support surfaces, optionally used in combination with the disclosed hybridization buffer formulations, may lead to improvements in CNR by a factor of 2, 5, 10, 100, or 1000-fold over those achieved using conventional supports and hybridization, amplification, and/or sequencing protocols. Although described here in the context of using fluorescence imaging as the read-out or detection mode, the same principles apply to the use of the disclosed low non-specific binding supports and nucleic acid hybridization and amplification formulations for other detection modes as well, including both optical and non-optical detection modes.

The disclosed low-binding supports, optionally used in combination with the disclosed hybridization and/or amplification protocols, yield solid-phase reactions that exhibit: (i) negligible non-specific binding of protein and other reaction components (thus minimizing substrate background), (ii) negligible non-specific nucleic acid amplification product, and (iii) provide tunable nucleic acid amplification reactions.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

In some embodiments, a fluorescence image of the surface exhibits a contrast-to-noise ratio (CNR) of at least 20 when a sample nucleic acid molecule or complementary sequences thereof are labeled with a Cyanine dye-3 (Cy3) fluorophore, and when the fluorescence image is acquired using an inverted fluorescence microscope (e.g., Olympus IX83) with a 20×0.75 NA objective, a 532 nm light source, a bandpass and dichroic mirror filter set optimized for 532 nm excitation and Cy3 fluorescence emission, and a camera (e.g., Andor® sCMOS®, Zyla® 4.2) under non-signal saturating conditions while the surface is immersed in a buffer (e.g., 25 mM ACES, pH 7.4 buffer).

### EXAMPLES

The following examples are meant to be illustrative and can be used to further understand embodiments of the present disclosure and should not be construed as limiting the scope of the present teachings in any way.

### Example 1: Enzyme-Based Dehybridization

In a series of hybridization-dehybridization experiments various formulations of enzyme-based dehybridization reagents were tested and compared against conventional chemical-based dehybridization reagents.

The hybridization reagent contained 150 mM NaCl, 50 mM MES (pH 5.5); and 1 M guanidinium chloride. Hybridization was conducted at 55° C. for 9-10 minutes. This hybridization reagent was used for testing the conventional and enzyme-based dehybridization reagents.

The conventional dehybridization reagent contained 40% formamide, 5% PEG, 2M guanidinium chloride, and 20 mM MES (pH 5.5). Dehybridization was conducted at 55° C. for 5 minutes, 37° C. for 5 minutes, and 20° C. for 5 minutes.

Numerous enzyme-based formulations were tested. Generally, the enzyme-based reagents were prepared with water as a solvent and included: T7 exonuclease gene 6 (e.g., from New England Biolabs® (e.g., catalog No. M0263S) or Thermo Fisher Scientific® (e.g., catalog No. 70025Z10KU); a pH buffering agent; a monovalent salt; a divalent salt; and an enzyme stabilizer. Alternatively, lambda exonuclease was tested.

The enzyme-based reagents included T7 exonuclease at a concentration of about 10-200 U/mL. In some embodiments, the monovalent salt comprises potassium, for example potassium acetate. In some embodiments, the divalent salt comprises magnesium, for example magnesium acetate. In some embodiments, the enzyme stabilizer comprises a protein comprising bovine serum albumin (BSA), beta-lactoglobulin (e.g., from bovine milk), beta-casein (e.g., from bovine milk) or ovalbumin. In some embodiments, the enzyme-bases reagent has a pH of about 6-9, or about 7-9. In some embodiments, the enzyme-based reagent further comprises NaCl at a concentration suitable to reduce 5′ flap endonuclease activity of the T7 exonuclease. The enzyme-based reagent included NaCl at a concentration of about 20-150 mM, or about 20-40 mM, or about 40-60 mM, or about 60-80 mM, or about 80-100 mM, or about 100-120 mM, or about 120-150 mM. Dehybridization using the enzyme-based reagent was conducted at different temperatures including about 25-35° C. or about 30-50° C. Dehybridization using the enzyme-based reagent was conducted for different times, including 5-15 minutes.

Flowcells having immobilized concatemers were prepared by conducting on-support rolling circle amplification using circularized library molecules carrying a sequence-of-interest and universal adaptor sequences. Sequencing primers in hybridization reagent were flowed onto the flowcells and incubated under conditions suitable to hybridize the sequencing primers to the universal sequencing primer binding sites on the concatemers to form template/primer duplexes. Sequencing polymerases and detectably labeled multivalent molecules were flowed onto the flowcells under conditions suitable for the polymerases to bind the template/primer duplexes to form complexed polymerases, and for the detectably labeled multivalent molecules to bind the complexed polymerases to form detectable complexes. The conditions also inhibited incorporation of the nucleotide units of the multivalent molecules by including a non-catalytic divalent cation. The flowcells were washed to remove unbound multivalent molecules. The washed flowcells were imaged to detect the detectable complexes.

The detectable complexes were removed from the immobilized concatemers by flowing onto the flowcells the chemical-based dehybridization reagent or one of the test enzyme-based dehybridization reagents, under conditions suitable to dissociate the sequencing primers from the concatemer template molecules along with the detectably labeled multivalent molecules and sequencing polymerases. The flowcells were washed to remove the dissociated sequencing primers, multivalent molecules and polymerases. The washed flowcells were imaged to detect any remaining detectable complexes.

Three cycles of hybridization-dehybridization and an extra hybridization step were conducted as follows: Hyb1/Dehyb1; Hyb2/Dehyb2; Hyb3/Dehyb3; and Hyb4. The results are shown in FIGS. 11-15.

### INCORPORATION BY REFERENCE

All publications and patent applications mentioned in this specification are herein incorporated by reference to the same extent as if each individual publication or patent application was specifically and individually indicated to be incorporated by reference.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A method for sequencing a nucleic acid template molecule, comprising:
a) providing at least one single-stranded nucleic acid template molecule immobilized to a support, wherein the at least one single-stranded nucleic acid template molecule is covalently attached or hybridized to a capture primer immobilized to the support;
b) hybridizing at least one first sequencing primer to a first region of the nucleic acid template molecule and conducting one or more cycles of sequencing reactions, thereby generating at least one first extension duplex comprising a first sequencing read product hybridized to the template molecule; and
c) contacting the first extension duplex with a double-stranded DNA specific enzyme having 5′ to 3′ exonuclease activity under a condition suitable to degrade the first sequencing read product and retain the template molecule, thereby removing at least a portion of the first sequencing read product from the first region of the template molecule; and
d) washing the retained template molecule at a temperature between 22-27° C. to remove the double-stranded DNA specific enzyme having 5′ to 3′ exonuclease activity.

2. The method of claim 1, further comprising:
e) hybridizing at least one second sequencing primer to a second region of the same nucleic acid template molecule and conducting one or more sequencing reactions, thereby generating a second extension duplex comprising a second sequencing read product hybridized to the template molecule;
f) contacting the second extension duplex with a double-stranded DNA specific enzyme having 5′ to 3′ exonuclease activity under a condition suitable to degrade the second sequencing read product and retain the template molecule thereby removing at least a portion of the second sequencing read product from the second region of the template molecule; and
g) washing the retained template molecule at a temperature between 22-27° C. to remove the double-stranded DNA specific enzyme having 5′ to 3′ exonuclease activity.

3. The method of claim 2, further comprising:
h) hybridizing at least one third sequencing primer to a third region of the same nucleic acid template molecule and conducting one or more sequencing reactions thereby generating a third extension duplex comprising a third sequencing read product hybridized to the template molecule;
i) contacting the third extension duplex with a double-stranded DNA specific enzyme having 5′ to 3′ exonuclease activity under a condition suitable to degrade the third sequencing read product and retain the template molecule, thereby removing at least a portion of the third sequencing read product from the third region of the template molecule; and
j) washing the retained template molecule at a temperature between 22-27° C. to remove the double-stranded DNA specific enzyme having 5′ to 3′ exonuclease activity.

4. The method of claim 3, further comprising; repeating steps (h), (i) and (j) using:
(i) a fourth sequencing primer that hybridizes to a fourth region of the same nucleic acid template molecule;
(ii) a fifth sequencing primer that hybridizes to a fifth region of the same nucleic acid template molecule;
(iii) a sixth sequencing primer that hybridizes to a sixth region of the same nucleic acid template molecule;
(iv) a seventh sequencing primer that hybridizes to a seventh region of the same nucleic acid template molecule;
(v) an eighth sequencing primer that hybridizes to an eighth region of the same nucleic acid template molecule;
(vi) a ninth sequencing primer that hybridizes to a ninth region of the same nucleic acid template molecule; and/or
(vii) a tenth or more sequencing primer that hybridizes to a tenth or more region of the same nucleic acid template molecule.

5. The method of claim 1, wherein the double-stranded DNA specific enzyme having 5′ to 3′ exonuclease activity comprises a T7 exonuclease enzyme encoded by T7 phage gene 6.

6. The method of claim 1, wherein the at least one single-stranded nucleic acid template molecule comprises:
a) one copy of a sequence-of-interest generated via bridge amplification, or
b) two or more tandem copies of a sequence-of-interest generated via rolling circle amplification.

7. The method of claim 1, wherein the at least one single-stranded nucleic acid template molecule comprises at least one uridine nucleotide.

8. The method of claim 1, wherein the at least one single-stranded nucleic acid template molecule lacks a uridine nucleotide.

9. The method of claim 8, wherein the at least one single-stranded nucleic acid template molecule comprises at least one sequence-of-interest and at least one universal adaptor sequence.

10. The method of claim 9, wherein the at least one universal adaptor sequence is selected from:
a first surface primer binding site (or a complementary sequence thereof) which can hybridize to at least a portion of an immobilized first surface primer;
a second surface primer binding site (or a complementary sequence thereof) which can hybridize to at least a portion of an immobilized second surface primer;
a first sequencing primer binding site (or a complementary sequence thereof) which can hybridize to at least a portion of a forward sequencing primer;
a second sequencing primer binding site (or a complementary sequence thereof) which can hybridize to at least a portion of a reverse sequencing primer;
a first amplification primer binding site (or a complementary sequence thereof) which can hybridize to at least a portion of a forward amplification primer;
a second amplification primer binding site (or a complementary sequence thereof) which can hybridize to at least a portion of a reverse amplification primer;
a first sample index sequence;
a second sample index sequence;
a first compaction oligonucleotide binding site (or a complementary sequence thereof) which can hybridize to at least a portion of a first compaction oligonucleotide;
a second compaction oligonucleotide binding site (or a complementary sequence thereof) which can hybridize to at least a portion of a second compaction oligonucleotide;
a first unique molecular tag sequence;
a second unique molecular tag sequence; or
any combination of two or more universal adaptor sequences.

11. The method of claim 1, wherein the first sequencing read product of step b) comprises the first sequencing primer joined to a first polynucleotide generated by a polymerase-catalyzed primer extension reaction, wherein the first polynucleotide comprises a sequence complementary to at least a portion of the at least one single-stranded nucleic acid template molecule.

12. The method of claim 2, wherein the second sequencing read product of step e) comprises the second sequencing primer joined to a second polynucleotide generated by a polymerase-catalyzed primer extension reaction, wherein the second polynucleotide comprises a sequence complementary to at least a portion of the at least one single-stranded nucleic acid template molecule.

13. The method of claim 3, wherein the third sequencing read product of step h) comprises the third sequencing primer joined to a third polynucleotide generated by a polymerase-catalyzed primer extension reaction, wherein the third polynucleotide comprises a sequence complementary to at least a portion of the at least one single-stranded nucleic acid template molecule.

14. The method of claim 10, wherein the order of the sequencing the at least one single-stranded nucleic acid template molecule comprises:
i) a) sequencing the sequence-of-interest; and
b) sequencing the first sample index after sequencing the sequence-of-interest;
ii) a) sequencing only a portion of the sequence-of-interest,
b) sequencing the first sample index after sequencing the sequence-of-interest, and
c) sequencing the full length of the sequence-of-interest after sequencing the first sample index;
iii) a) sequencing the sequence-of-interest;
b) sequencing the first sample index after sequencing the sequence-of-interest; and
c) sequencing the second sample index after sequencing the first sample index;
iv) a) sequencing only a portion of the sequence-of-interest;
b) sequencing the first sample index after sequencing the sequence-of-interest;
c) sequencing the second sample index after sequencing the first sample index; and
d) sequencing the full length of the sequence-of-interest after sequencing the second sample index;
v) a) sequencing the first sample index; and
b) sequencing the sequence-of-interest after sequencing the first sample index;
vi) a) sequencing the first sample index; and
b) sequencing only a portion of the sequence-of-interest after sequencing the first sample index
vii) a) sequencing the first sample index;
b) sequencing the second sample index after sequencing the first sample index; and
c) sequencing the sequence-of-interest after sequencing the second sample index;
viii) a) sequencing the first sample index;
b) sequencing the second sample index after sequencing the first sample index; and
c) sequencing only a portion of the sequence-of-interest after sequencing the second sample index;
ix) a) sequencing the first sample index;
b) sequencing the sequence-of-interest after sequencing the first sample index; and
c) sequencing the second sample index after sequencing the sequence-of-interest;
x) a) sequencing the first sample index;
b) sequencing only a portion of the sequence-of-interest after sequencing the first sample index; and
c) sequencing the second sample index after sequencing the sequence-of-interest; or
xi) a) sequencing the first sample index;
b) sequencing only a portion of the sequence-of-interest after sequencing the first sample index;
c) sequencing the second sample index after sequencing the sequence-of-interest; and
d) sequencing the full length of the sequence-of-interest after sequencing the second sample index.

15. The method of claim 1, wherein the conducting one or more cycles of sequencing reactions comprises conducting one or more cycles of a sequencing reaction with a sequencing polymerase and a plurality of detectably labeled nucleotide analogs.

16. The method of claim 15, wherein the detectably labeled nucleotide analogs comprise:
a) individual nucleotides comprising an aromatic nucleo-base, a five-carbon sugar moiety, 1-10 phosphate groups, and a fluorophore, or
b) individual nucleotides comprising an aromatic nucleo-base, a five-carbon sugar moiety having a removable chain terminating group at the 3′ carbon sugar position, 1-10 phosphate groups, and a fluorophore.

17. The method of claim 16, wherein the removable chain terminating group of the 3′ carbon sugar position comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group, and
wherein the removable chain terminating moiety is cleavable with a chemical compound to generate an extendible 3′ OH moiety on the sugar group.

18. The method of claim 15, wherein the plurality of detectably labeled nucleotide analogs comprises:
a) one type of nucleotide selected from the group consisting of dATP, dGTP, dCTP, dTTP and dUTP, or
b) two or more types of nucleotides selected from the group consisting of dATP, dGTP, dCTP, dTTP and dUTP.

19. The method of claim 1, wherein the conducting one or more cycles of sequencing reactions comprises conducting one or more cycles of a two-stage sequencing reaction, wherein
(i) the first stage of the two-stage sequencing reaction comprises sequencing with a first plurality of sequencing polymerases and a plurality of detectably labeled multivalent molecules, and
(ii) the second stage comprises sequencing with a second plurality of sequencing polymerases and a plurality of unlabeled nucleotide analogs, and
wherein individual detectably labeled multivalent molecules in the plurality comprise (1) a core, (2) a plurality of nucleotide arms, and (3) at least one fluorophore, wherein individual nucleotide arms comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, and wherein the linker is attached to the nucleotide unit.

20. The method of claim 19, wherein the at least one single-stranded nucleic acid template molecule comprises a concatemer molecule, and wherein the first stage of the two-stage sequencing reaction comprises forming a plurality of binding complexes comprising the steps:
a) binding a first sequencing primer, a first sequencing polymerase, and a first multivalent molecule to a first portion of the concatemer template molecule, thereby forming a first binding complex, wherein a first nucleotide unit of the first multivalent molecule binds to the first sequencing polymerase;
b) binding a second sequencing primer, a second sequencing polymerase, and the first multivalent molecule to a second portion of the same concatemer template molecule, thereby forming a second binding complex, wherein a second nucleotide unit of the first multivalent molecule binds to the second sequencing polymerase, wherein the binding of steps (a) and (b) are conducted under a condition suitable to inhibit polymerase-catalyzed incorporation of the bound first and second nucleotide units in the first and second binding complexes, respectively, wherein the first and second binding complexes which include the same multivalent molecule form an avidity complex;
c) detecting the first and second binding complexes on the same concatemer template molecule; and
d) identifying the first nucleotide unit in the first binding complex, thereby determining the sequence of the first portion of the concatemer template molecule, and identifying the second nucleotide unit in the second binding complex, thereby determining the sequence of the second portion of the concatemer template molecule.
